(19) United States
(12) Patent Application Publication
(10) Pub. No.: US 2026/0356914 A1
(43) Pub. Date: Nov. 12, 2026

(54) TREATMENT OF CANCER WITH DRQ POLYPEPTIDES

(71) Applicants: Oregon Health & Science University, Portland, OR (US); The United States Government as represented by the Department of Veterans Affairs, Washington, DC (US); Yeda Research and Development Co. Ltd., Rehovot (IL)

(72) Inventors: Arthur A. Vandenbark, Portland, OR (US); Rajan Kulkarni, Lake Oswego, OR (US); Roberto Meza-Romero, Beaverton, OR (US); Bianca Pellegrino, Rehovot (IL); Idit Shachar, Rehovot (IL)

(73) Assignees: Oregon Health & Science University, Portland, OR (US); The United States Government as represented by the Department of Veterans Affairs, Washington, DC (US); Yeda Research and Development Co. Ltd., Rehovot (IL)

(21) Appl. No.: 19/488,403

(22) PCT Filed: May 24, 2024
(86) PCT No.: PCT/US2024/031149

§ 371 Date: Nov. 26, 2025

Related U.S. Application Data
(60) Provisional application No. 63/469,261, filed on May 26, 2023.

(51) Int. Cl. C07K 14/705; A61K 38/00; A61K 45/06; A61P 35/00; C07K 14/47

(52) U.S. Cl. CPC C07K 14/70539; A61K 45/06; A61P 35/00; C07K 14/4713; A61K 38/00

(57) ABSTRACT

Methods of treating a subject with cancer with a recombinant polypeptide including an antigenic peptide covalently linked to a DRα1 domain or portion thereof comprising a glutamine residue at a position corresponding to amino acid 45 of SEQ ID NO: 1 or SEQ ID NO: 2 are provided. In some examples, the subject is resistant to immune checkpoint blockade treatment and/or has a tumor that does not express a BRAF mutation.

### CROSS REFERENCE TO RELATED APPLICATION

This application claims the benefit of and priority to U.S. Provisional Application No. 63/469,261, filed May 26, 2023, the entire contents of which are hereby incorporated by reference.

### ACKNOWLEDGMENT OF GOVERNMENT SUPPORT

This invention was made with government support under AI122574 awarded by the National Institutes of Health. The government has certain rights in the invention.

### FIELD

This application relates to methods of treating cancer with DRQ polypeptides including an antigenic peptide and a modified DRα1 domain.

### SEQUENCE LISTING

The Sequence Listing is submitted as an XML file in the form of the file named “Sequence.xml” (7,000 bytes), which was created on May 24, 2024, which is incorporated by reference herein.

### BACKGROUND

Melanoma is a deadly skin cancer whose incidence has been increasing. New therapies involving immune checkpoint blockade (ICB) have increased survival; however, these ICB therapies are only 30-40% effective as monotherapy. Overall survival increases to 57% with anti-PD-1 plus anti-CTLA-4, though this has significant side effects that not every patient can tolerate. The remainder either do not respond (primary resistance) or initially respond but stop (secondary resistance). Secondary resistance ranges from 22-60% of initial responders. While new drugs and personalized vaccines are being developed, these patients do not otherwise have good treatment options, particularly those without BRAF mutations.

Glioblastoma (GBM) is the most common, highly invasive and aggressive primary malignant brain tumor and the patient's prognosis with glioblastoma is extremely poor, with overall survival averaging 8 to 15 months. The current course of treatment involves a maximal safe resection followed by radiotherapy and a 6 to 12 month course of chemotherapy with the alkylating agent temozolomide. This treatment has only slightly improved the median survival by approximately two months in people 40 years of age or older. In addition, treatment with ICB has failed to demonstrate activity or efficacy in primary malignant gliomas.

Breast cancer (BC) is the most frequent malignancy affecting women worldwide [1]. BC is a heterogeneous disease composed of different subtypes. The classification of BC depends on the expression of three biomarkers: estrogen receptors (ERs), progesterone receptors (PRs), and human epidermal growth factor receptor 2 (HER2). BCs that are negative for ER, PR, and HER2 are known as triple-negative breast cancer (TNBC) [2]. TNBC is regarded as the most aggressive BC subtype, and its clinical features include high invasiveness, high metastatic potential, the propensity to relapse, and poor prognosis [3,4]. TNBC has been shown to feature a unique microenvironment (ME). TNBC cells manipulate their ME to become immunosuppressive, making this tumor a good candidate for immunotherapy [5]. Dendritic cells (DCs) are antigen-presenting cells (APCs) essential for the regulation of innate and adaptive immune responses. The multiple functions of DCs in immune regulation mirror their complexity and their heterogeneous subsets with different lineages, locations, phenotypes, and functional plasticity [6]. DCs play an important role in tumor immunity and are able to cross-present tumor-associated antigens (TAAs) to T-cells. By manipulating their microenvironment, tumor cells can perturb DC functions, reduce T cell activation, and potentially, the induction of T cell tolerance to TAAs [7]. The tumor can also empower immune-regulatory transcriptional programs that limit the DC-mediated production of pro-inflammatory cytokines and increase the release of IL-10 and indoleamine dioxygenase-1 (IDO1), which facilitate immunosuppression. DCs that produce IL-10 enforce T-cell anergy and are termed tol-DCs. IL-10 expression in DCs is considered a tolerogenic signature resulting in the induction of Tregs [8]. B cells also play an important role in the tumor ME. In addition to their role in the regulation of the humoral immune responses and their ability to produce antibodies and cytokines, some B cell populations, known as Bregs, have regulatory properties that are crucial for the maintenance of immune tolerance [9]. Bregs exert their function predominantly via the release of IL-10. The immune suppressive function of Bregs involves multiple mechanisms, including skewing T-cell differentiation, induction, and maintenance of Tregs, as well as suppression of pro-inflammatory cells [10].

CD74 (Ii chain) is a non-polymorphic type II transmembrane protein expressed mostly on the surface of APCs, and was initially thought to function solely as an MHC class II chaperone [11]. A small portion of the CD74 molecules undergo post-translational modifications that enable their cell surface expression [12]. Cell surface CD74 serves as a receptor for ligands of the Macrophage Migration Inhibitory Factor (MIF) family that includes the cytokines MIF-1 (MIF) and MIF-2/D-dopachrome tautomerase (DDT) [13]. Upon MIF binding, CD74 forms a cell surface complex with CD44, which is essential for the MIF-induced signaling cascade. The signaling pathway involves Syk tyrosine kinase and PI3K/Akt activation, which leads to CD74 intramembrane cleavage and the release of the CD74 intracellular domain (CD74-ICD). CD74-ICD translocates to the nucleus, where it induces cell proliferation and survival of B cells [14-20]. Interestingly, both MIF and CD74 have been associated with tumor progression and metastasis. It was reported that MIF mRNA is over-expressed in various tumors [21, 22] and MIF has also been associated with the growth of malignant cells [23]. Many studies have demonstrated that CD74 expression is upregulated in various cancers [24-29] including chronic lymphocytic leukemia (CLL) [30, 31] and correlates with poor prognosis. In particular, CD74 expression is upregulated in patients with TNBC compared to other breast cancer subtypes, and is associated with lymph node metastasis, leading to a worsening of the overall survival [32, 33]. CD74 expression has also been suggested to serve as a prognostic factor in many of these cancers, with higher relative expression of CD74 behaving as a marker of tumor progression [34]. Although it is well known that CD74 plays a crucial role in hematological malignancies such as CLL and that its expression correlates with a poor prognosis, its function and mechanism in TNBC are incompletely understood.

Thus, there remains a need for improved treatments for melanoma, glioblastoma, breast cancer, as well as other cancers, particularly in tumors that do not respond to ICB therapy or subjects who are resistant to ICB therapy.

### SUMMARY

Provided herein are methods of treating cancer using DRQ polypeptides or nucleic acids and compositions including DRQ polypeptides or nucleic acids. In some aspects, the methods include administering to a subject with cancer a therapeutically effective amount of a recombinant polypeptide including an antigenic peptide covalently linked to a DRα1 domain or portion thereof including a glutamine residue at a position corresponding to amino acid 50 of SEQ ID NO: 1 or SEQ ID NO: 2, or a nucleic acid encoding the recombinant polypeptide. In some examples, the recombinant polypeptide further includes a linker or spacer between the antigenic peptide and the DRα1 domain. In one example, the linker includes a first glycine-serine spacer, a thrombin cleavage site, and a second glycine-serine spacer.

In some aspects, the antigenic peptide is myelin oligodendrocyte glycoprotein (MOG)-35-55, for example, human or mouse MOG-35-55. In some examples, the recombinant polypeptide includes or consists of the amino acid sequence of SEQ ID NO: 1 or SEQ ID NO: 2.

In some aspects, the subject is administered about 0.1 mg/kg to about 10 mg/kg of the recombinant polypeptide. In additional aspects, the cancer is a solid tumor, for example, melanoma, glioblastoma, or breast cancer. In further aspects, the cancer of the subject does not express a BRAF V600 mutation, the subject with cancer is resistant to immune checkpoint blockade therapy, or both.

The disclosed methods may further include administering one or more additional therapies to the subject. In some examples, the one or more additional therapies include one or more of surgery, radiation, chemotherapy, and immunotherapy. In particular examples, the immunotherapy includes immune checkpoint blockade therapy.

The foregoing and other features of the disclosure will become more apparent from the following detailed description, which proceeds with reference to the accompanying figures.

### BRIEF DESCRIPTION OF THE DRAWINGS

FIG. 1 is a Kaplan-Meier plot showing decreased overall survival in advanced melanoma patient samples that exhibit higher MIF expression.

FIG. 2 presents a graph showing decreased MIF production in B16F10 cells grown with DRmQ compared to vehicle control.

FIG. 3 shows growth curves of an intradermal B16F10 mouse melanoma model treated with vehicle control or DRmQ (P<0.05 with unpaired two-tailed t-test).

FIG. 4 presents a photomicrograph showing the effects of DRQ treatment on immune infiltrates in B16F10 melanoma tumors.

FIG. 5 presents a plot illustrating that DRQ (top) increases infiltration of TRP2-reactive CD8+ cells, compared to vehicle control (bottom) as measured by TRP2-PE tetramer.

FIG. 6 presents a Western blot showing pSTAT3, pAKT, and pERK in B16F10 cells incubated with nothing (no tx), vehicle, or 50 μg DRQ for 1 hour.

FIG. 7A presents line and bar graphs 5-day measurements of tumor progression regulated by CD74.

FIG. 7B presents a photograph of tumors removed and measured from euthanized mice after 21 days of growth.

FIG. 7C presents a bar graph plotting tumor volume, with each dot representing an excised tumor.

FIG. 7D presents a bar graph plotting the mean and SD percentage of dendritic cells (DC) in the tumor site (WT n=11; CD74^{-/-} n=8).

FIG. 7E presents a bar graph plotting CD80⁺ DCs as a percentage out of total DCs (WT n=11; CD74^{-/-} n=8).

FIG. 7F presents a bar graph plotting tolerogenic DCs that were analyzed for IL-10 among total DCs.

FIG. 7G presents a graph showing the mean and SD percentage of FOXP3⁺ T cells out of total CD4⁺ T cells (WT n=8; CD74KO n=8).

FIG. 7H presents a graph showing the mean and SD percentage of IL-10⁺ B cells out of total B cells (WT n=8; CD74 KO n=7).

FIG. 7I presents a graph showing the mean and SD percentage of IFN-γ⁺ T cells out of total CD8⁺ T cells (WT n=5; CD74KO n=4).

FIG. 7J presents a photograph of tumors measured after 21 days of growth.

FIG. 7K presents a graph of tumor volumes measured after 21 days of growth.

FIG. 7L presents a graph representing IL-10⁺ DCs analyzed as the percentage of total DCs (for PBS n=8; DRQ n=8).

FIG. 7M presents a graph representing IL-12⁺ DCs analyzed as the percentage of total DCs (for PBS n=4; DRQ n=4).

FIG. 7N presents a graph representing the mean and SD percentage of IL-10⁺ B cells out of total B cells (for PBS n=7; DRQ n=8).

FIG. 7O presents a graph representing the mean and SD percentage of Tregs out of total CD4⁺ cells (for PBS n=7; DRQ n=8).

FIG. 8A presents a graph representing MIF-CD74 axis regulation of tol-DC and Breg expansion.

FIG. 8B presents a graph representing the fold change of Bregs expansion out of B cells, under the different culture conditions (WT n=21; CD74KO n=21).

FIG. 8C presents a graph of the mean and SD percentage of IL10⁺ DCs out of total dendritic cells (WT+PBS n=12; WT+MIF n=13).

FIG. 8D presents a graph of the mean and SD percentage of Bregs out of B cells (WT+PBS n=12; WT+MIF n=12).

FIG. 8E presents a graph of MIF mRNA levels analyzed by qRT-PCR. Graphs present fold change (siRNA/siCtrl) of the chosen gene (n=3).

FIG. 8F presents a graph of the mean and SD percentage of IL 10⁺ DCs out of total dendritic cells (WT+Sico E0771 n=13; WT+siMIF n=13).

FIG. 8G presents bar graphs representing the mean and SD percentage of Bregs out of B cells (WT+Sico E0771 n=14; WT+siMIF n=14).

FIG. 9A provides a graph of tumor sizes were measured and recorded (n=22) after 21 days.

FIG. 9B provides a graph of IL-10⁺ DCs analyzed as the percentage of total DCs (for the WT n=8; conditional CKO n=7).

FIG. 9C provides a graph of the mean and SD percentage of IL-12⁺ dendritic cells out of total DCs (for the WT n=5; CKO n=5).

FIG. 9D provides a graph of the mean and SD percentage of IL-10 B cells out of total CD19⁺ cells (for the WT n=6; CKO n=6).

FIG. 9 E provides a graph showing the mean and SD percentage of IL10⁺ DCs out of total dendritic cells (WT n=5; C KO n=5).

FIG. 9F provides a graph showing the mean and SD percentage of Bregs out of B cells. (WT n=7; CKO n=7).

FIG. 10A provides a line graph of tumor size recorded from female 6-weeks-old CD11c-Cre×CD74flox×CD74 flox mice every five days following injection of 5*10⁵ E0771 cells into each of the 4^{th} mammary pads.

FIG. 10B presents a photograph of tumor sizes were measured and recorded (n=44) after 21 days.

FIG. 10C presents a graph of the tumor volumes with each dot representing an individual tumor.

FIG. 10D presents a graph of IL-10⁺ DCs analyzed as a percentage of total DCs (WT n=13; conditional CKO n=12).

FIG. 10E presents a graph of the mean and SD percentage of IL-10⁺ B cells out of total B cells (WT n=12; CKO n=11).

FIG. 10F presents a graph of the mean and SD percentage of Tregs out of total CD4⁺ cells (WT n=13; CKO n=10).

FIG. 10G presents a graph of the mean and SD percentage of the tumor-infiltrating CD8⁺ T-cells out of total CD3⁺ T cells (WT n=13; CKO n=12).

FIG. 10H presents a graph of the mean and SD percentage of the IFN-γ releasing T-cells out of total CD8⁺ T-cells (WT n=4; CKO n=4).

FIG. 10I presents a graph of the mean and SD percentage of PD1⁺ T-cells out of total CD8⁺ T-cells (WT n=4; C KO n=4).

FIG. 10J presents a graph of the mean and SD percentage of IL10⁺ DCs out of total dendritic cells. (WT n=8; conditional CD74 KO n=8).

FIG. 10K presents a graph of the mean and SD percentage of Bregs out of B cells (WT n=8; conditional CD74^{-/-} n=8).

FIG. 11A presents a graph of the mean and SD percentage of IL 10⁺ dendritic cells out of total dendritic cells, under the different culture conditions (WT DCs+WT B cells n=8; CD75T4 KO DCs+WT B cells n=8; WT DCs+CD74 KO B cells n=8; CD74^{-/-} DCs+CD74 KO B cells n=8).

FIG. 11B presents a graph of the mean and SD percentage of IL 10⁺ B cells out of total B cells, under the different culture conditions (WT DCs+WT B cells n=8; CD74 KO DCs+WT B cells n=8; WT DCs+CD74 KO B cells n=8; CD74 KO DCs+CD74 KO B cells n=8).

FIG. 11C presents a graph depicting the mean and SD percentage of IL10⁺ B cells out of total B cells (B cells+WT DCs CM n=10; B cells+CD74 cKO DCs CM n=10)

FIG. 11D presents a graph depicting the mean and SD percentage of IL 10⁺ dendritic cells out of total DCs (DCs+B cells treated with IGg n=8; DCs+B cells treated with LN-2 n=8).

FIG. 11E presents a graph depicting the mean and SD percentage of proliferating CD8⁺ T cells under the different culture conditions (WT n=14; CD74 KO n=15).

FIG. 11F presents a graph depicting the mean and SD percentage of FOXP3⁺ CD4⁺ T cells under the indicated culture conditions (WT n=9; CD74/n=11).

FIG. 11G presents a graph depicting depicts the percentage of IFN-γ⁺ CD8⁺ T cells (WT n=14; CD74 KO n=15).

FIG. 12A presents a graph representing the fold change in mRNA levels of SP1 following injections of 5*10⁵ E0771 cells into each of the 4^{th} mammary pads of 6 weeks old female C57BL/6 mice.

FIG. 12B presents a graph representing the fold change in mRNA levels of IL1β following injections of 5*10⁵ E0771 cells into each of the 4^{th} mammary pads of 6 weeks old female C57BL/6 mice.

FIG. 12C presents a graph representing fold change of SP1 in WT and CD74 KO mice (n=6).

FIG. 12D presents a graph representing fold change of IL1β in WT and CD74 KO mice (n=6).

FIG. 13A presents a graph representing the mean and SD percentage of IL10⁺ dendritic cells out of total DCs (DCs+vehicle=18; DCs+IL1β n=20).

FIG. 13B presents a graph representing the mean and SD percentage of IL10⁺ B cells out of total B cells and IL10⁺ DCs out of total dendritic cells (B cells+DCs treated with PBS, n=5; B cells+DCs treated with IL-1β, n=5).

FIG. 13C presents a graph depicting the mean and SD percentage of IL 10⁺ B cells out of total B cells (B cells treated with PBS n=10; B cells treated with IL-1β n=10; B cells+DCs treated with PBS n=10; B cells+DCs treated with IL-1β n=10).

FIG. 13D presents a graph representing the percentage of enrichment of the input (the amount of DNA pulled down by using the antibody of interest in the ChIP reaction, relative to the amount of starting material-input sample) (n=4).

FIG. 14A presents a graph representing the fold change of the expansion of IL10⁺ dendritic cells out of total DCs (DCs+DMSO=10; DCs+MIT n=10), with each dot representing a mouse.

FIG. 14B presents a graph depicting the fold change of IL10⁺ B cells out of total B cells and IL10⁺ DCs out of total dendritic cells (B cells treated with DMSO n=5; B cells treated with mithramycin n=5).

FIG. 14C presents a graph representing the mean and SD percentage of IL10⁺ B cells out of total B cells (B cells treated with DMSO n=10; B cells treated with MIT n=10, B cells+DCs treated with DMSO n=10; B cells+DCs treated with MIT n=10).

FIG. 14D presents a graph representing the percentage of enrichment of the input (the amount of DNA pulled down by CD74 antibody in the ChIP reaction, relative to the amount of starting material-input sample) (n=5).

FIG. 14E presents a graph representing the binding of CD74-ICD in DCs activated with mrMIF (n=5).

FIG. 14F presents a graph representing the percentage of enrichment of the input (the amount of DNA pulled down by using SP1 antibody in the ChIP reaction, relative to the amount of starting material-input sample) in DCs activated with mrMIF, DRQ or vehicle (n=14).

FIG. 14G presents a graph representing the mean and SD percentage of IL-1β⁺ DCs out of total DCs (DCs treated with DMSO=8, DCs treated with MIT=8).

FIG. 15A presents a box plot analysis depicting the relative expression level of CD74 in several immune cell populations.

FIG. 15B presents a graph showing the mean and SD percentage of CD74 expression on B cells in the spleen and in the TME (B cells in the spleen n=4; B cells in the TME n=4).

FIG. 15C presents a graph showing the mean and SD percentage of CD74 expression on dendritic cells in the spleen and in the TME (dendritic cells in the spleen n=4; dendritic cells in the TME n=4).

FIG. 16A represents analysis of DC cells for CD11c expression after excluding LY6-C+, F4/80+ and CD19. B cells were analyzed for CD19 after excluding LY6-C+, F4/80+ and CD11c.

FIG. 16B provides a plot representing analysis of IL-10+ expression on DCs was measured by comparing the non-activated for DCs (mono WT) 1,24 with the ones activated with PIM.

FIG. 16C provides a plot representing analysis of IL-10+ expression on DCs was measured by comparing the non-activated for DCs (WT) 52,6 with the ones activated with PIM.

FIG. 16D provides a plot representing analysis of IL-10+ expression on DCs was measured by comparing the non-activated for DCs (mono cKO) 1,22 with the ones activated with PIM.

FIG. 16E provides a plot representing analysis of IL-10+ expression on DCs was measured by comparing the non-activated for DCs (CKO) 3,13 with the ones activated with PIM.

FIG. 16F provides a plot representing IL-12+ expression on DCs measured by comparing the non-activated for sample subset (mono WT) 6,89 with the ones activated with PIM.

FIG. 16G provides a plot representing IL-12+ expression on DCs measured by comparing the non-activated for sample subset (WT) 7,95 with the ones activated with PIM.

FIG. 16H provides a plot representing IL-12+ expression on DCs measured by comparing the non-activated for sample subset (mono KO) 7,33 with the ones activated with PIM.

FIG. 16I provides a plot representing IL-12+ expression on DCs measured by comparing the non-activated for sample subset (KO) 13, 9 with the ones activated with PIM.

FIG. 16J provides a plot representing IL-10+ expression on B cells measured by comparing the nonactivated for either WT and CD74−/− samples with the ones activated with PIM.

FIG. 16K provides a plot representing IL-10+ expression on B cells measured by comparing the nonactivated for either WT and CD74−/− samples with the ones activated with PIM.

FIG. 16L provides a plot representing IL-10+ expression on B cells measured by comparing the nonactivated for either WT and CD74−/− samples with the ones activated with PIM.

FIG. 16M provides a plot representing IL-10+ expression on B cells measured by comparing the nonactivated for either WT and CD74−/− samples with the ones activated with PIM.

FIG. 17A presents a graph representing the frequency of IL-10+ B cells out of total B cells (WT n=8; CD74−/− n=7).

FIG. 17B presents a graph representing the frequency of IL-10+ DCs in the tumor site (WT n=14; CD74−/− n=11) for DC cells were analyzed for CD45, CD11c, and IL-10 expression after excluding LY6-C+, F4/80+ and CD19+ cells.

FIG. 17C presents a graph representing the frequency of IL-10+ macrophages after excluding monocytes and DCs (WT n=5; CD74−/− n=5).

FIG. 17D presents a graph representing the frequency of CD4+ T-cells (WT n=9; CD74−/− n=8).

FIG. 17E presents a graph representing the frequency of CD8+ T-cells (WT n=9; CD74−/− n=8).

FIG. 17F presents a graph representing the frequency of IFN-γ+ T cells out of total CD8+ T cells (WT n=5; CD74−/− n=4).

FIG. 17G presents a graph representing the frequency of IFN-γ+ T cells out of total CD8+ T cells (WT n=5; CD74−/− n=4).

FIG. 17H presents a graph representing the frequency of PD1+ T cells out of total CD8+ T cells (WT n=5; CD74−/− n=4).

FIG. 17I presents a graph representing the frequency of CD62L+ T cells out of total CD8+ T cells (WT n=5; CD74−/− n=5).

FIG. 18A presents a graph representing the frequency of IL-10+ B cells out of total B cells (PBS n=7; DRQ n=8).

FIG. 18B presents a graph representing the frequency of IL-10+ DCs in the tumor site (PBS n=8; DRQ n=8).

FIG. 18C presents a graph representing the frequency of CD4+ T-cells (PBS n=4; DRQ n=5).

FIG. 18D presents a graph representing the frequency of FOXP3+ T cells out of total CD4+ T cells (PBS n=4; DRQ n=5).

FIG. 18E presents a graph representing the frequency of CD8+ T-cells (PBS n=4; DRQ n=5).

FIG. 19A presents a graph representing the mean and SD percentage of monocytes out of total live cells (WT n=5, cKO n=5).

FIG. 19B presents a graph representing the mean and SD percentage of macrophages out of total live cells (WT n=5, cKO n=5).

FIG. 19C presents a graph representing the mean and SD percentage of B cells out of total live cells (WT n=5, cKO n=5).

FIG. 19D presents a graph representing the percentage of DCs/live cells.

FIG. 19E presents a graph representing the mean and SD percentage of CD74 expression on monocytes.

FIG. 19F presents a graph representing the mean and SD percentage of CD74 expression on macrophages.

FIG. 19G presents a graph representing the mean and SD percentage of CD74 expression on B cells.

FIG. 19H presents a graph representing the mean and SD percentage of CD74 expression on DCs.

FIG. 19I presents a graph representing the mean and SD percentage of CD4+ T cells out of total live cells (WT n=6; cKO n=6).

FIG. 19J presents a graph representing the mean and SD percentage of CD8+ T cells out of total live cells (WT n=6; cKO n=6).

FIG. 19K presents a graph representing the mean and SD percentage of CD62L+ T cells out of total CD4+ T cells (WT n=6; cKO n=6).

FIG. 19L presents a graph representing the mean and SD percentage of CD103+ T cells out of total CD8+ T cells (WT n=6; cKO n=6).

FIG. 19M presents a graph representing the mean and SD percentage of FOXP3+ T cells out of total CD4+ T cells (WT n=4; cKO n=4).

FIG. 19N presents a graph representing the expression of CD74 in the CD4+ populations.

FIG. 19O presents a graph representing the expression of CD74 in the C84+ populations.

FIG. 20A presents a plot representing the dead cells excluded from analysis by Zombie Live/Dead staining.

FIG. 20B presents a plot representing that macrophages and monocytes were gated for F4/80 and LY-6c, respectively.

FIG. 20C presents a plot representing that the double negative population was analyzed for CD19 and CD11c to detect DC and B cells.

FIG. 20D presents a plot representing the analysis of DCs obtained in FIG. 20C for CD26.

FIG. 20E presents a plot representing that the CD45+ population was gated for CD26 as a dendritic cell marker.

FIG. 20F presents a plot representing that the CD45+ population was gated for CD26 as a dendritic cell marker.

FIG. 20F presents a plot representing that CD26+ DCs were analyzed for F4/80.

FIG. 20G presents a plot representing that CD26+ DCs were analyzed for LY-6c.

FIG. 20H presents a plot representing that CD26+ DCs were analyzed for CD19.

FIG. 20I presents a plot representing that the CD45+ population was gated for CD64 as a macrophage marker.

FIG. 20J presents a plot representing that CD64+ macrophages were analyzed for CD19 and LY-6c expression.

FIG. 21A presents a graph representing the mean and SD percentage of IL-10+ monocytes out of total monocytes (WT n=4, cKO n=4) 21 days following injection of 5*105 E0771 cells into each of the 4th mammary pads of female 6-week-old CD23-Cre×CD74flox×CD74 flox mice.

FIG. 21B presents a graph representing the mean and SD percentage of IL-12+ monocytes out of the total population (WT n=4, cKO n=4).

FIG. 21C presents a graph representing the mean and SD percentage of IL-10+ macrophages out of total macrophages (WT n=4, cKO n=4).

FIG. 21D presents a graph representing the mean and SD percentage of IL-12+ macrophages out of total (WT n=4, cKO n=4).

FIG. 22A presents a graph representing the frequency of IL-10+ B cells out of total B cells (WT n=7; cKO n=7) 21 days following injection of 5*105 E0771 cells into each of the 4th mammary pads of female 6-week-old CD23-Cre×CD74flox×CD74 flox mice.

FIG. 22B presents a graph representing the frequency of IL-10+ DCs in the tumor site (WT n=8; CD74 cKO n=7).

FIG. 22C presents a graph representing the frequency of CD 4+ T-cells (WT n=7; CD74 cKO n=8).

FIG. 22D presents a graph representing the frequency of FOXP3+ T cells out of total CD4+ T cells (WT n=4; CD74 cKO n=4).

FIG. 22E presents a graph representing the frequency of CD 8+ T-cells (WT n=8; CD74 cKO n=7).

FIG. 22F presents a graph representing the frequency of PD1+ T cells out of total CD8+ T cells (WT n=4; CD74 cKO n=4).

FIG. 22G presents a graph representing the frequency of IFN-γ+ T cells out of total CD8+ T cells (WT n=4; CD74 cKO n=4).

FIG. 22H presents a graph representing the frequency of CD103+ T cells out of total CD8+ T cells (WT n=4; CD74 cKO n=4).

FIG. 22I presents a graph representing the frequency of CD62L+ T cells out of total CD8+ T cells (WT n=4; CD74 cKO n=4).

FIG. 23A represents a visualization of the Ingenuity Pathway Analysis (IPA) where the relevant pathways are shown ordered by significance (p-value), calculated in IPA by right-tailed Fischer's exact t-test.

FIG. 23B represents an IPA Upstream Regulator Analysis used to predict the upstream regulators responsible for the gene expression changes observed.

FIG. 23C presents a depiction of gene interactions, where some genes are upregulated, and some are suppressed.

### SEQUENCES

Any nucleic acid and amino acid sequences listed herein are shown using standard single letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is the amino acid sequence of an exemplary DRhQ polypeptide.
DRhQ includes the antigenic peptide human MOG-35-55 (bold), a spacer (underlined),
and a modified DRα1 domain (italics). The L50Q mutation in the DRα1 portion is
shown in bold italic:
MEVGWYRPPFSRVVHLYRNGKGGGGSLVPRGSGGGGIKEEHVIIQAEF
YQNPDQSGEFMFDFDGDEIFHVDMAKKETVWRLEEFGRFASFEAQGALAN
IAVDKANLEIMTKRSNYTPITN
SEQ ID NO: 2 is the amino acid sequence of an exemplary DRmQ polypeptide.
DRmQ includes the antigenic peptide mouse MOG-35-55 (bold), a spacer (underlined),
and a modified DRα1 domain. The L50Q mutation in the DRα1 portion is shown in
bold italic:
MEVGWYRSPFSRVVHLYRNGKGGGGSLVPRGSGGGGIKEEHVIIQAEF
YQNPDQSGEFMDFDGDEIFHVDMAKKETVWRLEEFGRFASFEAQGALANI
AVDKANLEIMTKRSNYTPITN
SEQ ID NO: 3 is an exemplary nucleic acid encoding a DRhQ polypeptide:
ATGGAAGTTGGTTGGTACCGTCCCCCGTTCTCCCGTGTTGTTCACCTG
TACCGTAACGGTAAAGGAGGTGGAGGCTCACTAGTGCCCCGAGGCTC
TGGAGGTGGAGGCATCAAAGAAGAACATGTGATCATCCAGGCCGAG
TTCTATCAGAATCCTGACCAATCAGGCGAGTTTATGTTTGACTTTGAT
GGTGATGAGATTTTCCATGTGGATATGGCAAAGAAGGAGACGGTCTG
GCGGCTTGAAGAATTTGGACGATTTGCCAGCTTTGAGGCTCAAGGTG
CATTGGCCAACATAGCTGTGGACAAAGCCAACTTGGAAATCATGACA
AAGCGCTCCAACTATACTCCGATCACCAATTAA
SEQ ID NO: 4 is an exemplary nucleic acid encoding a DRmQ polypeptide:
ATGGAAGTTGGTTGGTACCGTTCCCCGTTCTCCCGTGTTGTTCACCTG
TACCGTAACGGTAAAGGAGGTGGAGGCTCACTAGTGCCCCGAGGCTC
TGGAGGTGGAGGCATCAAAGAAGAACATGTGATCATCCAGGCCGAG
TTCTATCAGAATCCTGACCAATCAGGCGAGTTTATGTTTGACTTTGAT
GGTGATGAGATTTTCCATGTGGATATGGCAAAGAAGGAGACGGTCTG
GCGGCTTGAAGAATTTGGACGATTTGCCAGCTTTGAGGCTCAAGGTG
CATTGGCCAACATAGCTGTGGACAAAGCCAACTTGGAAATCATGACA
AAGCGCTCCAACTATACTCCGATCACCAATTAA
SEQ ID NO: 5 is a myelin basic protein (MBP) 85-99 peptide:
PVVHFFKNIVTPRT

### DETAILED DESCRIPTION

CD74 is a highly conserved molecule with multiple functions. The CD74 “CLIP” peptide plays a key intracellular role in loading antigenic peptides onto class II

MHC molecules. The macrophage migration inhibitory factor (MIF) binding region for CD74 is extracellular, does not overlap with CLIP and can be expressed by multiple cell types independently of its role within the MHC II complex, including in melanoma. Upon binding MIF, CD74 undergoes phosphorylation of its cytosolic domain to initiate downstream signal transduction, through activation of the Lck protein tyrosine kinase, followed by activation of MAPK kinase (MEK), leading to phosphorylation of ERK1/2. In addition, AKT and PI3K pathways can be upregulated by MIF.

Expression of CD74 is significantly upregulated in different cell types in various cancers [24-29], and is correlated with tumor progression. MIF, a pro-inflammatory cytokine that serves as the ligand of CD74, was previously shown to be necessary for the immunosuppressive ME in melanoma and glioblastoma [48, 49]. It was found to be overexpressed in several types of tumors, including TNBC [50]. The MIF-CD74 axis has been shown to play pivotal roles, not only in initiating an oncogenic signaling pathway but also provoking inflammatory responses, thereby promoting tumor growth and an immunosuppressive milieu [51-53]. However, the function of CD74 in the environment of the immune cells in the TNBC ME has not been described and the function of DCs and Bregs in this context were not analyzed.

The present disclosure demonstrates that TNBC cells secrete MIF, which binds CD74 expressed on DCs and B cells, inducing a phenotypic switch from immunogenic to tolerogenic. Blocking CD74 leads to a reduced tumor load due to the elevated activity of the tumor-infiltrating immune cells. This anti-tumor phenotype is mainly caused by decreased IL-10 secretion, which results in a global decrease of the suppressive Bregs, Tregs, and tol-DCs in the TME.

Bregs and tol-DCs in the TNBC ME can affect each other, enhancing IL-10 release via a positive feedback loop [54]. A feedback loop between tol-DCs and Bregs was previously demonstrated in several processes such as T cell clonal anergy and Treg expansion, highlighting the complexity of the mediators involved in the generation and maintenance of tolerance. This crosstalk can be a double-edged sword, beneficial for example in the case of autoimmunity, but harmful in the case of cancer [55-57].

The present disclosure shows that DCs significantly govern Breg expansion via CD74-induced pathways. However, Bregs are not able to control DC differentiation, supporting the hypothesis that DCs are the major players among tumor-infiltrating immune cells. The tumor-infiltrating immune cells communicate among themselves with a synergistic effect [58].

The present disclosure also demonstrates that DCs control B cells and induce their immunosuppressive phenotype by regulating the expression of several genes involved in the immune response, resulting in an upregulation of IL-10 expression. The activation of CD74 results in the binding of CD74-ICD, which serves as a transcription regulator [20, 47] to the SP1 and the IL-1β promotors in DCs and elevates their mRNA expression, suggesting that besides its role in B cells, CD74-ICD serves as a transcription regulator in DCs. Higher expression levels of SP1 [43], reduced expression of the pro-inflammatory cytokine IL-1β [59], and elevation of IL-10 secretion from the DC population, are associated with immunosuppression in TNBC. Accordingly, these DCs are less immunogenic and enhance the expansion of immune-suppressive cells [46].

The embodiments of the present disclosure define the role of IL-1β specifically in the DC population within the tumor microenvironment, where it is known to regulate their inflammatory activity through IL-12 production. DCs lacking CD74, are able to promote an immunogenic response via IL-1β release, resulting in the inhibition of the expansion of Bregs. According to the present disclosure, the effect of DCs-mediated IL-1β secretion appears to be dominant compared to its direct effect on the B cells themselves, suggesting that IL-1β releasing DCs hamper the expansion of the Breg population. Studies on the role of IL-1β in regulatory B cells were performed mainly in autoimmune diseases, in which the environment and cytokines are completely different from those in the TME [60]. It is known that cytokines have different functions in different contexts and cells, and therefore it is suggested that IL-1β might function differently in immune cells in the context of autoimmunity or cancer.

Therefore, blocking the CD74-induced pathway downregulates SP1 expression in DCs, resulting in an upregulated IL-1β secretion, which strongly reduces Breg expansion, leading to the activation of the immune response. The higher IL-1β secretion from DCs, is determined by both the direct effect of CD74 on its promotor, and the indirect effect of the MIF-CD74-SP1 axis. Hence, CD74 might serve as a novel therapeutic target in melanoma as well as in triple-negative breast cancer.

Disclosed herein are methods of treating cancer with a class of peptide agents that bind CD74 and block extracellular signaling by MIF, in part by blocking downstream signaling through phosphorylated extracellular-related kinase (pERK1/2). The agent, termed DRQ, is a partial MHC class II protein construct linked to myelin oligodendrocyte glycoprotein peptide (MOG-35-55). DRQ binds CD74 and competitively inhibits MIF signaling (Meza-Romero et al., Metab Brain Dis. 34:153-164, 2019). Preliminary data suggest that DRQ at the doses tested can selectively modulate the tumor microenvironment (TME) to promote anti-tumor activity while not materially affecting other functions. While blocking the CD74/MIF axis has been proposed in cancer (Kang et al., Nat. Rev. Rheumatol. 15:427-437, 2019), many molecules evaluated had dose-limiting toxicity. A main advantage of DRQ is its lack of dose-limiting toxicity up to maximum feasible dose. As demonstrated herein, DRQ has a potent anti-tumor effect and although there are other activities of MIF beyond binding to CD74, the main effect of DRQ is on CD74/MIF signaling. DRQ could thus provide an alternative treatment for individuals that fail currently approved treatments, particularly those who are BRAF wild type.

### I. TERMS

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a polypeptide” includes singular or plural polypeptides and can be considered equivalent to the phrase “at least one polypeptide.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

To facilitate review of the various aspects, the following explanations of terms are provided:

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” or “antigenic peptide” refers to a site on an antigen to which B and/or T cells respond. In one aspect, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 8 amino acids (such as about 8-50 or 8-23 amino acids) in a unique spatial conformation. Methods of determining spatial conformation of epitopes include, for example, x-ray crystallography and two-dimensional nuclear magnetic resonance.

An antigen can be a tissue-specific antigen, or a disease-specific antigen. These terms are not exclusive, as a tissue-specific antigen can also be a disease-specific antigen. A tissue-specific antigen is expressed in a limited number of tissues, such as a single tissue. A tissue-specific antigen may be expressed by more than one tissue, such as, but not limited to, an antigen that is expressed in the central or peripheral nervous system.

BRAF: Also known as B-Raf proto-oncogene, serine/threonine kinase. A member of the RAF family of serine/threonine kinases involved in regulating MAP kinase/ERK signaling pathway. Mutations in BRAF (such as V600E or V600K) are the most frequently identified mutations in melanoma and also occur in other cancers, such as colorectal cancer, thyroid cancer, and non-small cell lung carcinoma. Nucleic acid and protein sequences for BRAF are publicly available. For example, GenBank Accession Nos. NM_004333 and NM_001374258 disclose exemplary human BRAF nucleic acid sequences, and GenBank Accession Nos. NP_004324 and NP 001361187 disclose exemplary human BRAF amino acid sequences. Each of these sequences is incorporated herein by reference as present in GenBank on May 26, 2023.

CD74: Also known as CD74 molecule, Major Histocompatibility Complex class II invariant chain or MHC Class II gamma chain, or Ii. CD74 is a chaperone regulating antigen presentation. It is also a cell surface receptor for macrophage migration inhibitory factor (MIF). Nucleic acid and protein sequences for CD74 are publicly available. For example, GenBank Accession Nos. NM_001025158, NM_004355, and NM 001025159 disclose exemplary human CD74 nucleic acid sequences, and GenBank Accession Nos. NP_001020329, NP_004346, and NP_001020330 disclose exemplary human CD74 amino acid sequences. Similarly, GenBank Accession Nos. NM_001042605 and NM_010545 disclose exemplary mouse CD74 nucleic acid sequences, and GenBank Accession Nos. NP_001036070 and NP_034675 disclose exemplary mouse CD74 amino acid sequences. Each of these sequences is incorporated herein by reference as present in GenBank on May 26, 2023.

Control: A “control” refers to a sample or standard used for comparison with an experimental sample. In some aspects, the control is a sample obtained from a healthy subject or population of healthy subjects. In other aspects, the control is a historical control or standard reference value or range of values (such as a previously tested control sample, such as a group of samples that represent baseline or normal values). In further examples, the control is from a subject prior to treatment (such as prior to treatment with a DRQ polypeptide).

Domain: A discrete part of an amino acid sequence of a polypeptide or protein that can be equated with a particular function. For example, the a and B polypeptides that constitute a MHC class II molecule are each recognized as having two domains, α1, α2 and β1, β2, respectively. The various domains are typically joined by linking amino acid sequences. In one aspect, the entire domain sequence is included in a recombinant molecule by extending the sequence to include all or part of the linker or the adjacent domain. For example, when selecting the α1 domain of an MHC class II molecule, the selected sequence may extend from amino acid residue number 1 of the a chain, through the entire α1 domain to amino acid 84 at the carboxy terminus of the α1 domain. The precise number of amino acids in the various MHC molecule domains varies depending on the species of mammal, as well as between classes of genes within a species. The selection of a sequence for use in a recombinant molecule requires maintenance of the domain function rather than a precise structural definition based on the number of amino acids. One of ordinary skill in the art will appreciate that domain function may be maintained even if somewhat less than the entire amino acid sequence of the selected domain is utilized. For example, a number of amino acids at either the amino or carboxy termini of the α1 domain may be omitted without affecting domain function. In other examples, substitution of amino acids within a domain may increase or decrease the binding affinity of the domain. For example, the substitution of glutamine for leucine at position 50 of the DRhQ construct can increase its binding affinity for CD74.

Immune checkpoint blockade: A cancer immunotherapy that targets regulators of the immune system that dampen the immune response. Examples of checkpoint proteins found on T cells or cancer cells include PD-1, PD-L1, CTLA-4, BTLA, and TIM-3. These proteins inhibit T cells from killing cancer cells. Checkpoint inhibitors include agents that target molecules such as lymphocyte activation gene 3 (LAG3), T-cell immunoglobulin and mucin-domain containing-3 (TIM-3), CTLA-4 (e.g., ipilimumab, YERVOY®), PD-1 (e.g., nivolumab, OPDIVO® and pembrolizumab, KETRUDA®), and PD-L1.

Inhibiting or treating a disease: “Inhibiting” a disease refers to inhibiting the full development of a disease, for example in a person who is known to have a predisposition to a disease such as cancer. Inhibition of a disease can span the spectrum from partial inhibition to substantially complete inhibition of the disease, for example in a subject who has a disease or disorder or is at risk of developing a disease or disorder. In some examples, the term “inhibiting” refers to reducing or delaying the onset or progression of a disease. “Treating” a disease refers to a therapeutic intervention that ameliorates a sign or symptom of a disease or pathological condition, such as a sign or symptom of cancer. A subject to be administered an effective amount of the pharmaceutical compound to inhibit or treat the disease or disorder can be identified by standard diagnosing techniques for such a disorder, for example, symptoms, basis of family history, or risk factor to develop the disease or disorder.

Linker: A molecule that covalently links two molecules (such as two polypeptides). Linkers (such as a peptide linker or a chemical linker) may be included in the recombinant MHC polypeptides of the present disclosure, for example between an α1 domain and an antigenic peptide. Peptide linker sequences, which are generally between 2 and 25 amino acids in length (such as 5-10, 10-15, 15-20, or 20-25 amino acids), include, but are not limited to, the glycine (4)-serine spacer described by Chaudhary et al. (Nature 339:394-397, 1989). Similarly, chemical linkers (such as thiol bonds or crosslinking agents) can also be used.

MHC Class II: MHC Class II molecules are formed from two noncovalently associated proteins, the a chain and the B chain. The a chain comprises α1 and α2 domains, and the β chain comprises β1 and 2 domains. The cleft into which the antigen fits is formed by the interaction of the α1 and β1 domains. The α2 and β2 domains are transmembrane Ig-fold like domains that anchor the a and B chains into the cell membrane of the APC. MHC Class II complexes, when associated with antigen (and in the presence of appropriate co-stimulatory signals) stimulate CD4 T-cells. The primary functions of CD4 T-cells are to initiate the inflammatory response, to regulate other cells in the immune system, and to provide help to B cells for antibody synthesis.

Pharmaceutically acceptable carriers: Remington: The Science and Practice of Pharmacy, Adejare (Ed.), Academic Press, London, United Kingdom, 23^{rd} Edition (2021) describes compositions and formulations suitable for pharmaceutical delivery of the polypeptides and nucleic acids herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, trehalose, glycerol or the like as a vehicle. For solid compositions (such as powder, pill, tablet, or capsule forms), conventional non-toxic solid carriers can include, for example, pharmaceutical grades of mannitol, trehalose, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Recombinant: A recombinant nucleic acid or polypeptide is one that has a sequence that is not naturally occurring or has a sequence that is made by an artificial combination of two or more otherwise separated segments of sequence. This non-natural sequence or artificial combination is often accomplished by chemical synthesis or by the artificial manipulation of isolated segments of nucleic acids, e.g., by genetic engineering techniques.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are. Polypeptides or domains thereof that have a significant amount of sequence identity and function the same or similarly to one another—for example, the same protein in different species—can be called ‘homologs.’ Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-244, 1988; Higgins & Sharp, Comput. Appl. Biosci. 5:151-153, 1989; Corpet et al., Nucl. Acids Res. 16, 10881-90, 1988; Huang et al., Comput. Appl. Biosci. 8, 155-65, 1992; and Pearson, Methods Mol. Biol. 24:307-331, 1994. Altschul et al. (J. Mol. Biol. 215:403-410, 1990) presents a detailed consideration of sequence alignment methods and homology calculations. The NCBI Basic Local Alignment Search Tool (BLAST) is available from several sources, including the National Center for Biotechnology Information (NCBI, Bethesda, MD) and on the Internet, for use in connection with the sequence analysis programs blastp, blastn, blastx, tblastn and tblastx.

Nucleic acid sequences that do not show a high degree of sequence identity may nevertheless encode similar amino acid sequences, due to the degeneracy of the genetic code. It is understood that changes in nucleic acid sequence can be made using this degeneracy to produce multiple nucleic acid molecules that all encode substantially the same protein.

Subject: Living multi-cellular vertebrate organisms, a category that includes both human and non-human mammals.

Therapeutically effective amount: A quantity of a specific substance sufficient to achieve a desired effect in a subject being treated. For instance, this can be the amount necessary to inhibit or suppress growth of a tumor. In one aspect, a therapeutically effective amount is the amount necessary to eliminate, reduce the size, or prevent metastasis of a tumor, or to increase progression-free survival and/or overall survival of the subject. When administered to a subject, a dosage will generally be used that will achieve target tissue concentrations (for example, in tumors) that has been shown to achieve a desired in vitro or in vivo effect (for example, in an animal model or a clinical trial).

### II. OVERVIEW

Clause 1: A method of treating a subject with cancer comprising administering to the subject a therapeutically effective amount of a recombinant polypeptide comprising an antigenic peptide covalently linked to a DRα1 domain or portion thereof comprising a glutamine residue at a position corresponding to amino acid 50 of SEQ ID NO: 1 or SEQ ID NO: 2; or a nucleic acid encoding the recombinant polypeptide.

Clause 2: The method of clause 1, wherein the recombinant polypeptide further comprises a linker between the antigenic peptide and the DRα1 domain.

Clause 3: The method of clause 2, wherein the linker comprises a first glycine-serine spacer, a thrombin cleavage site, and a second glycine-serine spacer.

Clause 4: The method of any one of clauses 1 to 3, wherein the antigenic peptide is myelin oligodendrocyte glycoprotein (MOG)-35-55 or myelin basic protein (MBP)-85-99.

Clause 5: The method of clause 4, wherein the MOG-35-55 is human or mouse MOG-35-55.

Clause 6: The method of any one of clauses 1 to 5, wherein the recombinant polypeptide comprises or consists of the amino acid sequence of SEQ ID NO: 1 or SEQ ID NO: 2.

Clause 7: The method of any one of clauses 1 to 6, wherein the subject is administered about 0.1 mg/kg to about 10 mg/kg of the recombinant polypeptide.

Clause 8: The method of any one of clauses 1 to 7, wherein the cancer is a solid tumor or a hematological malignancy.

Clause 9: The method of clause 8, wherein the solid tumor is melanoma, glioblastoma, or breast cancer.

Clause 10: The method of any one of clauses 1 to 9, wherein the cancer of the subject does not express a BRAF mutation.

Clause 11: The method of clause 10, wherein the cancer of the subject does not express a BRAF V600 mutation.

Clause 12: The method of any one of clauses 1 to 11, wherein the subject with cancer is resistant to immune checkpoint blockade therapy.

Clause 13: The method of any one of clauses 1 to 12, further comprising administering one or more additional therapies to the subject.

Clause 14: The method of clause 13, wherein the one or more additional therapies comprise one or more of surgery, radiation, chemotherapy, and immunotherapy.

Clause 15: The method of clause 14, wherein the immunotherapy comprises immune checkpoint blockade therapy.

### III. METHODS OF TREATING CANCER WITH DRQ

Methods of treating a subject with cancer with a DRQ polypeptide or nucleic acid are provided. The DRQ polypeptides include an antigenic peptide covalently linked to an MHC class II DRα1 domain or fragment thereof and do not include MHC class II α2, β1, or β2 domains, and include a substitution of glutamine (Q) for the leucine (L) present at the amino acid position corresponding to amino acid 50 of SEQ ID NO: 1 or SEQ ID NO: 2. In some aspects, the antigenic peptide included in the DRQ polypeptide is MOG-35-55. In particular examples, the MOG-35-55 is human MOG-35-55, and the DRQ polypeptide is referred to as DRhQ, or the MOG-35-55 is mouse MOG-35-55, and the DRQ polypeptide is referred to as DRmQ. In other examples, the antigenic peptide included in the DRQ polypeptide is myelin basic protein (MBP) 85-99 (e.g., SEQ ID NO: 5). Thus in some examples, the MOG-35-55 peptide (e.g., amino acids 1-21 of SEQ ID NO: 1 or SEQ ID NO: 2) is replaced with the MBP-85-99 peptide of SEQ ID NO: 5.

In some aspects, the DRQ polypeptide has at least 95% identity (such as at least 95%, 96%, 97%, 98%, 99%, or more identity) to the amino acid sequence of SEQ ID NO: 1 or SEQ ID NO: 2. In other aspects, the DRQ polypeptide includes or consists of the amino acid sequence of SEQ ID NO: 1 or SEQ ID NO: 2. In other aspects, the DRQ polypeptide is encoded by a nucleic acid having at least 90% identity (such as at least 90%, 91%, 92%, 93%, 94%, 95%, 96%, 97%, 98%, 99%, or more identity) to the nucleic acid sequence of SEQ ID NO: 3 or SEQ ID NO: 4. In other aspects, the DRQ polypeptide is encoded by a nucleic acid including or consisting of the nucleic acid sequence of SEQ ID NO: 3 or SEQ ID NO: 4.

In some aspects, the recombinant polypeptide (e.g., SEQ ID NO: 1 or SEQ ID NO: 2) can be expressed in prokaryotic or eukaryotic cells from a nucleic acid construct encoding the recombinant polypeptide (such as a nucleic acid construct including SEQ ID NO: 3 or SEQ ID NO: 4). Nucleic acid constructs (such as expression constructs) encoding the recombinant polypeptides may also include regulatory elements such as promoters, enhancers, and/or 3′ regulatory regions, the selection of which will be determined based upon the type of cell in which the protein is to be expressed. The constructs are introduced into a vector suitable for expressing the recombinant polypeptide in the selected cell type.

Numerous prokaryotic and eukaryotic systems are known for the expression and purification of polypeptides. For example, heterologous polypeptides can be produced in prokaryotic cells by placing a strong, regulated promoter and an efficient ribosome binding site upstream of the polypeptide-encoding construct. Suitable promoter sequences include the beta-lactamase, tryptophan (trp), phage T7, and lambda P_{L} promoters. Methods and plasmid vectors for producing heterologous proteins in bacteria or mammalian cells are known to one of ordinary skill in the art.

Suitable prokaryotic cells for expression of large amounts of proteins include Escherichia coli and Bacillus subtilis. Often, proteins expressed at high levels are found in insoluble inclusion bodies; methods for extracting proteins from these aggregates are known to one of ordinary skill in the art. Recombinant expression of recombinant polypeptides in prokaryotic cells may alternatively be conveniently obtained using commercial systems designed for optimal expression and purification of fusion proteins. Such fusion proteins typically include a tag that facilitates purification. Examples of such systems include: the pMAL protein fusion and purification system (New England Biolabs, Inc., Beverly, MA); the GST gene fusion system (Amersham Pharmacia Biotech, Inc., Piscataway, NJ); and the pTrcHis expression vector system (Invitrogen, Carlsbad, CA). Additional systems include the His6-tag (e.g., Roche Applied Science, Mannheim, Germany) or streptavidin binding peptide (e.g., Sigma-Aldrich, St. Louis, MO). For example, the pMAL expression system utilizes a vector that adds a maltose binding protein to the expressed protein. The fusion protein is expressed in E. coli. and the fusion protein is purified from a crude cell extract using an amylose column. If necessary, the maltose binding protein domain can be cleaved from the fusion protein by treatment with a suitable protease, such as Factor Xa. The maltose binding fragment can then be removed from the preparation by passage over a second amylose column.

The recombinant polypeptides can also be expressed in eukaryotic expression systems, including Pichia pastoris, Drosophila, Baculovirus and/or Sindbis expression systems produced by Invitrogen (Carlsbad, CA). Eukaryotic cells such as Chinese Hamster ovary (CHO), monkey kidney (COS), HeLa cells, 293 cells, Spodoptera frugiperda, and Saccharomyces cerevisiae may also be used to express recombinant polypeptides. Regulatory regions suitable for use in these cells include, for mammalian cells, viral promoters such as those from CMV, adenovirus or SV40, and for yeast cells, the promoter for 3-phosphoglycerate kinase or alcohol dehydrogenase.

The vectors can be introduced into recipient cells (such as eukaryotic cells) as pure DNA (transfection) by, for example, precipitation with calcium phosphate or strontium phosphate, electroporation, lipofection, DEAE dextran, microinjection, protoplast fusion, or microprojectile guns. Alternatively, the nucleic acid molecules can be introduced by infection with virus vectors. Systems are developed that use, for example, retroviruses, adenoviruses, or Herpes virus.

Pharmaceutical compositions that include a recombinant polypeptide or nucleic acid disclosed herein (such as an effective amount of a disclosed recombinant polypeptide or nucleic acid) can be formulated with an appropriate solid or liquid carrier, depending upon the particular mode of administration chosen. The pharmaceutically acceptable carriers and excipients useful in this disclosure include those known to one of ordinary skill in the art. See, e.g., Remington: The Science and Practice of Pharmacy, Adejare (Ed.), Academic Press, London, United Kingdom, 23^{rd} Edition (2021). For instance, parenteral formulations usually include injectable fluids that are pharmaceutically and physiologically acceptable fluid vehicles such as water, physiological saline, other balanced salt solutions, aqueous dextrose, glycerol or the like. For solid compositions (e.g., powder, pill, tablet, or capsule forms), conventional non-toxic solid carriers can include, for example, pharmaceutical grades of mannitol, lactose, starch, or magnesium stearate.

In addition to biologically-neutral carriers, pharmaceutical compositions to be administered can contain minor amounts of non-toxic auxiliary substances, such as wetting or emulsifying agents, preservatives, pH buffering agents, cyclodextrins, cryo-protectant sugars, or the like, for example trehalose, sodium acetate or sorbitan monolaurate. Excipients that can be included are, for instance, other proteins, such as human serum albumin or plasma preparations. The dosage form of the pharmaceutical composition will be determined by the mode of administration chosen. For instance, in addition to injectable fluids, topical, inhalation, oral and suppository formulations can be employed. Topical preparations can include eye drops, ointments, sprays, patches and the like. Inhalation preparations can be liquid (e.g., solutions or suspensions) and include mists, sprays and the like. Oral formulations can be liquid (e.g., syrups, solutions or suspensions), or solid (e.g., powders, pills, tablets, or capsules). Suppository preparations can also be solid, gel, or in a suspension form. Actual methods of preparing such dosage forms are known, or will be apparent, to one of ordinary skill in the art.

In some examples, the pharmaceutical composition may be administered by any mode that achieves its intended purpose. Amounts and regimens for the administration of the recombinant polypeptides or portion thereof (or a nucleic acid encoding such polypeptides) can be determined by the attending clinician. Effective doses for therapeutic application will vary depending on the nature and severity of the condition to be treated, the age and condition of the patient, and other clinical factors. Typically, the dose range will be from about 0.1 mg/kg body weight to about 10 mg/kg body weight. Other suitable ranges include doses of from about 0.1 mg/kg to about 5 mg/kg body weight, about 0.25 mg/kg to about 2.5 mg/kg body weight, or about 1 mg/kg to about 5 mg/kg body weight, for example, about 0.1 mg/kg, about 0.25 mg/kg, about 0.5 mg/kg, about 0.75 mg/kg, about 1 mg/kg, about 1.5 mg/kg, about 2 mg/kg, about 2.5 mg/kg, about 3 mg/kg, about 4 mg/kg, or about 5 mg/kg.

The pharmaceutical compositions that include a DRQ polypeptide or nucleic acid can be formulated in unit dosage form, suitable for individual administration of precise dosages. In one specific, non-limiting example, a unit dosage can contain from about 10 mg to about 1 g of the recombinant polypeptide (such as about 10 mg to about 50 mg, about 25 mg to about 250 mg, about 50 mg to about 500 mg, or about 100 mg to about 1 g). The amount of active compound(s) administered will be dependent on the subject being treated, the severity of the disorder being treated, and the manner of administration. Within these bounds, the formulation to be administered will contain a quantity of the active component(s) in amounts effective to achieve the desired effect in the subject being treated.

The dosing schedule may vary from daily to once every other month, depending on a number of clinical factors, such as the subject's condition and sensitivity to the administered composition. Examples of dosing schedules daily, every other day, three times/week, bi-weekly, weekly, two times/month (e.g., every two weeks), monthly (e.g., every 4 weeks), every 6 weeks, or every 8 weeks. In some examples, the treatment period is about 6 months, about 1 year, about 18 months, about 2 years, or more. In other examples, the treatment period continues until the subject no longer responds to the treatment, for example, the subject exhibits disease progression.

The recombinant DRQ polypeptides or nucleic acids can be administered to humans or other animals on whose tissues they are effective in various manners such as topically, orally, intravenously, intramuscularly, intraperitoneally, intranasally, intradermally, intrathecally, subcutaneously, via inhalation, or via suppository. In one example, the compounds are administered to the subject intravenously.

In some aspects, a composition including a DRQ polypeptide or nucleic acid is administered to a subject with cancer; for example, a subject with a solid tumor. Examples of solid tumors, include sarcomas (e.g., fibrosarcoma, myxosarcoma, liposarcoma, chondrosarcoma, osteogenic sarcoma, and other sarcomas), synovioma, mesothelioma, Ewing sarcoma, leiomyosarcoma, rhabdomyosarcoma, colon cancer, colorectal cancer, peritoneal cancer, esophageal cancer, pancreatic cancer, breast cancer (e.g., basal breast carcinoma, ductal carcinoma, lobular breast carcinoma, or triple-negative breast cancer), lung cancer, ovarian cancer, prostate cancer, liver cancer (e.g., hepatocellular carcinoma), gastric cancer, squamous cell carcinoma (e.g., head and neck squamous cell carcinoma), basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, hepatoma, bile duct carcinoma, choriocarcinoma, Wilms tumor, cervical cancer, fallopian tube cancer, testicular tumor, seminoma, bladder cancer, kidney cancer (e.g., renal cell cancer), melanoma, and CNS tumors (e.g., a glioma, glioblastoma, astrocytoma, medulloblastoma, craniopharyngioma, ependymoma, pinealoma, hemangioblastoma, acoustic neuroma, oligodendroglioma, meningioma, neuroblastoma, and retinoblastoma). Solid tumors also include tumor metastases (e.g., metastases to the lung, liver, brain, or bone). In particular examples, the subject has melanoma, glioblastoma, breast cancer, colon cancer, or lung cancer.

In other aspects a composition including a DRQ polypeptide or nucleic acid is administered to a subject with a hematological malignancy. Examples of hematological malignancies include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

In some aspects, the subject with cancer is a subject that has not responded to or no longer responds to (e.g., has or has developed resistance to) one or more cancer therapies. In some examples, the subject has or has developed resistance to immune checkpoint blockade treatment (such as treatment with one or more of ipilimumab, nivolumab, and pembrolizumab), such as has disease progression following treatment with one or more immune checkpoint blockade therapies. The resistance may be primary resistance (e.g., the subject does not respond) or secondary resistance (the subject initially responded, but then progressed). In some examples, the immune checkpoint blockade treatment was the first-line treatment.

In other examples, the subject (for example, a tumor of a subject) does not have a mutation in the BRAF gene (e.g., is BRAF wild type). BRAF mutations are most frequently found in melanoma, and are also found in colon cancers, rectal cancers, lung cancers, thyroid cancers, ovarian cancers, and brain cancers. BRAF mutations are known in the art and are described in Smiech et al., (Genes (Basel) 11:1342, 2020). In some examples, BRAF mutations include Class I mutations (V600 mutations, for example V600E, V600K, V600D, V600R, or V600M) which account for about 90% of all mutations. In other examples, BRAF mutations include Class II or Class III mutations, which account for the remaining mutations. Thus, in some examples, the subject does not have a BRAF V600 mutation (for example, their tumor does not express a BRAF V600 mutation). In other examples, the subject does not have a Class II or Class III BRAF mutation (for example, their tumor does not express a BRAF Class II or Class III mutation).

In additional aspects, the methods include selecting a subject that has primary or secondary resistance to immune checkpoint blockade treatment, does not have a BRAF mutation, or both. In some examples, the methods further include determining whether a tumor sample from the subject expresses a BRAF V600 mutation. A subject whose tumor does not express a BRAF V600 mutation may be selected for treatment.

In some aspects, treatment with DRQ results in an increase in progression-free survival, an increase in overall survival, or both, for example as compared with a subject not treated with DRQ. In other aspects, the progression-free survival is at least 2 months, at least 4 months, at least 6 months, at least 9 months, at least 12 months, at least 15 months, at least 18 months, at least 2 years, or more. In additional aspects, the overall survival is at least 6 months, at least 9 months, at least 12 months, at least 15 months, at least 18 months, at least 2 years, or more. In other aspects, treatment with DRQ results in decreased tumor size, decreased number of tumors, or decreased metastasis, for example as compared with a subject not treated with DRQ.

In some aspects, the DRQ polypeptide or nucleic acid is administered to the subject as a cancer monotherapy. In other examples, additional agents can be administered to the subject, such as a chemotherapeutic agent or immune checkpoint blockade therapy. These can be included in the disclosed pharmaceutical compositions or administered separately. In additional examples, surgical treatment and/or radiation can be administered to the subject. Administration of additional therapies may be sequential or simultaneous. A skilled clinician can select additional therapies to be administered, for example, based on the cancer being treated, the subject's response to prior therapies, the subject's condition, and other factors.

Examples of chemotherapeutic agents of use in the disclosed methods include alkylating agents, antimetabolites, natural products, or hormones and their antagonists. Examples of alkylating agents include nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine). Examples of antimetabolites include folic acid analogs (such as methotrexate), pyrimidine analogs (such as 5-FU or cytarabine), and purine analogs, such as mercaptopurine or thioguanine. Examples of natural products include vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Examples of miscellaneous agents include platinum coordination complexes (such as cis-diamine-dichloroplatinum II also known as cisplatin), substituted ureas (such as hydroxyurea), methyl hydrazine derivatives (such as procarbazine), and adrenocrotical suppressants (such as mitotane and aminoglutethimide). Examples of hormones and antagonists include adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of the most commonly used chemotherapy drugs include Adriamycin, Alkeran, Ara-C, BiCNU, Busulfan, CCNU, Carboplatinum, Cisplatinum, Cytoxan, Daunorubicin, DTIC, 5-FU, Fludarabine, Hydrea, Idarubicin, Ifosfamide, Methotrexate, Mithramycin, Mitomycin, Mitoxantrone, Nitrogen Mustard, Taxol (or other taxanes, such as docetaxel), Velban, Vincristine, VP-16, Gemcitabine, Herceptin, Irinotecan, Leustatin, Navelbine, Rituxan STI-571, Taxotere, Topotecan, Capecitabine), Zevelin and calcitriol. Non-limiting examples of immunomodulators that can be used include AS-101, bropirimine, gamma interferon, GM-CSF (granulocyte macrophage colony stimulating factor), IL-2, human immune globulin, IMREG, SK&F 106528, and TNF (tumor necrosis factor).

In other aspects, the additional chemotherapeutic agent can be an antibody. Exemplary monoclonal antibody therapies includes trastuzumab, alemtuzumab, atezolizumab, avelumab, bevacizumab, blinatumomab, cetuximab, daratumumab, ipilimumab, nivolumab, ofatumumab, panitumumab, pembrolizumab, pertuzumab, and rituximab. In particular examples, the antibody can be an immune checkpoint inhibitor, for example an antibody specifically binds PD-1, PD-L1, TIM-3, or CTLA-4.

Treatment regimens may also include combination with surgery, chemotherapy, radiation, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation. Exemplary chemotherapeutic agents include an anthracycline (e.g., doxorubicin (e.g., liposomal doxorubicin)). a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an m TOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

### EXAMPLES

The following examples are provided to illustrate particular features of certain aspects of the disclosure, but the scope of the disclosure should not be limited to those features exemplified.

### Example 1

### Details of Experimental Procedures and Figures

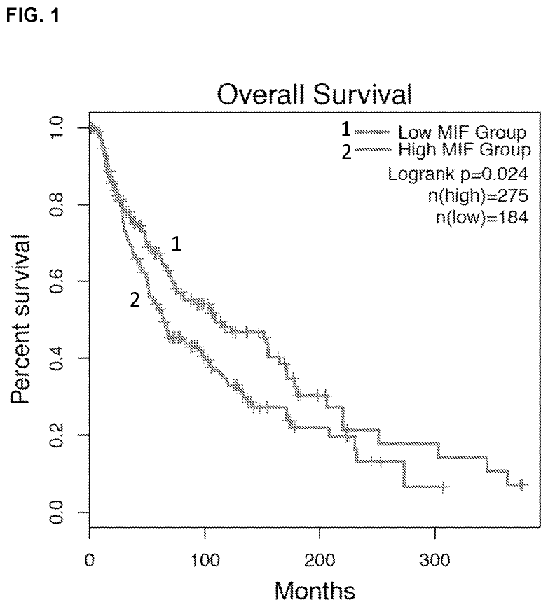

FIG. 1 is a Kaplan-Meier plot showing decreased overall survival in advanced melanoma patient samples that exhibit higher MIF expression. Data are from the TCGA skin cancer melanoma dataset (n=459, p=0.024 with log-rank test).

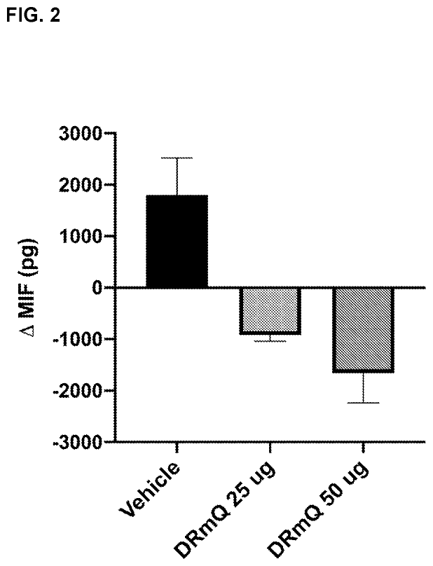

FIG. 2 is a graph showing decreased MIF production in B16F10 cells grown with DRmQ compared to vehicle control.

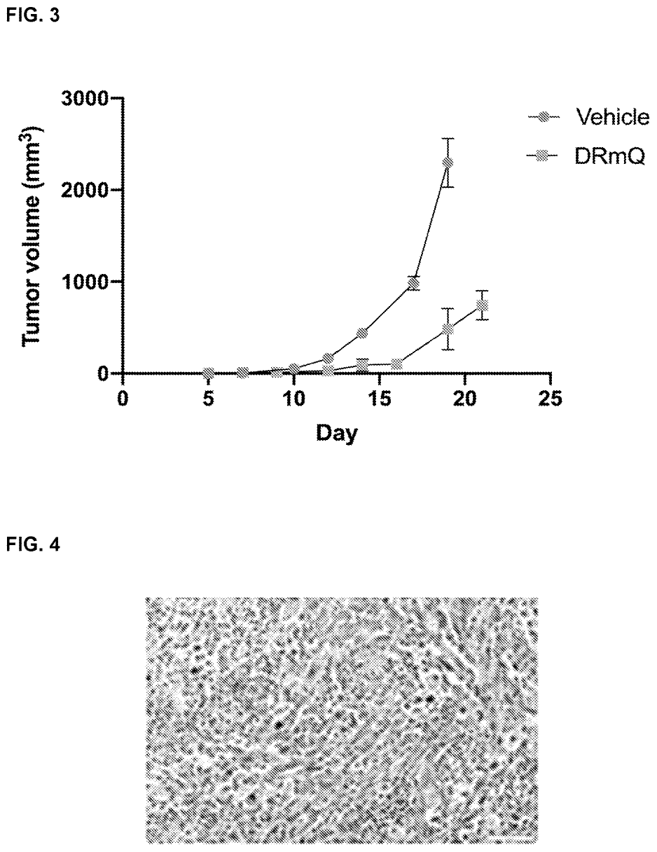

FIG. 3 shows growth curves of an intradermal B16F10 mouse melanoma model treated with vehicle control or DRmQ (P<0.05 with unpaired two-tailed t-test).

FIG. 4 show the effects of DRQ treatment on immune infiltrates in B16F10 melanoma tumors. The image is a photomicrograph illustrating that B16F10 melanoma tumor treated with DRQ demonstrates notable immune infiltrate (20× magnification, scale bar=50 μm). Based on flow cytometry analysis notable immune infiltrates within the total tumor cell population of DRQ treated tumors were observed (CD45+ as well as CD45+CD8+ dual positive).

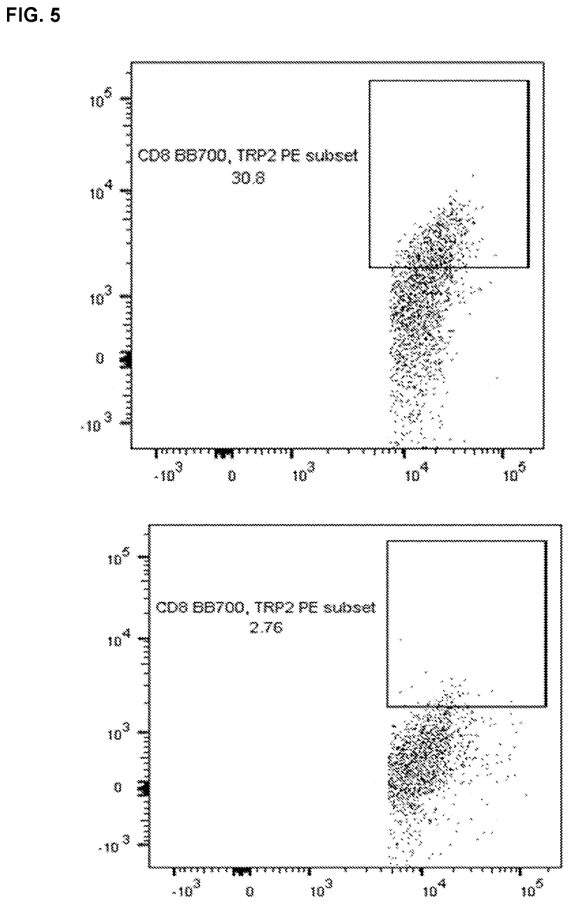

FIG. 5 illustrates that DRQ (top) increases infiltration of TRP2-reactive CD8+ cells, compared to vehicle control (bottom) as measured by TRP2-PE tetramer.

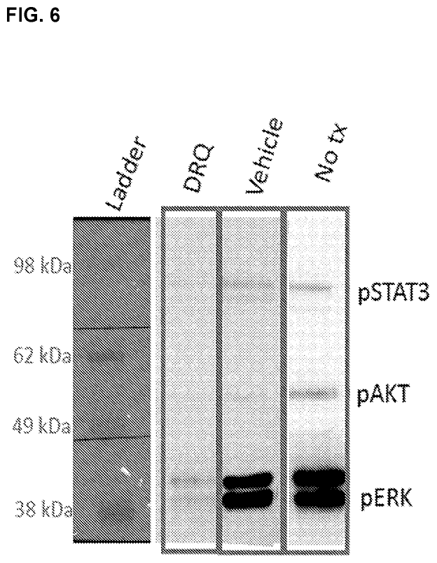

FIG. 6 is a Western blot showing pSTAT3, pAKT, and pERK in B16F10 cells incubated with nothing (no tx), vehicle, or 50 μg DRQ for 1 hour.

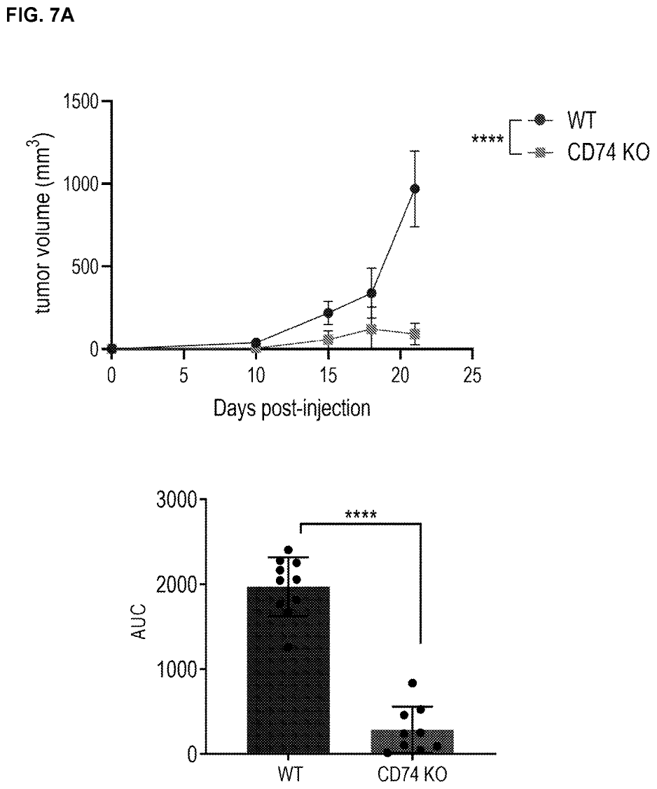

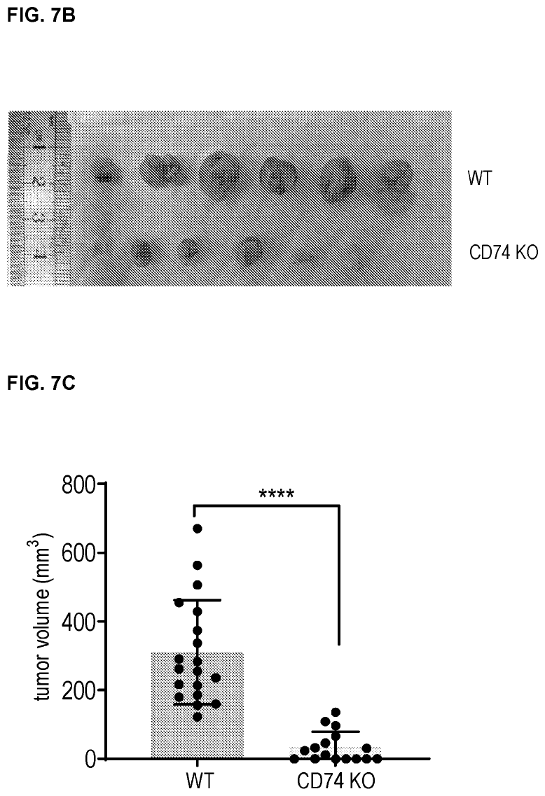

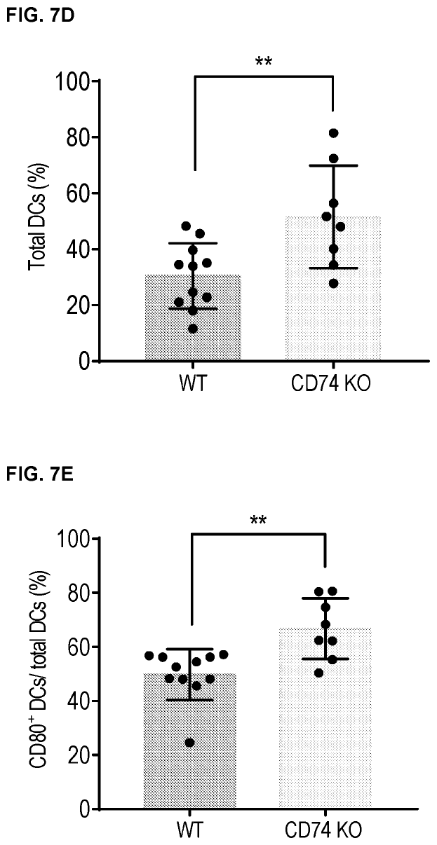

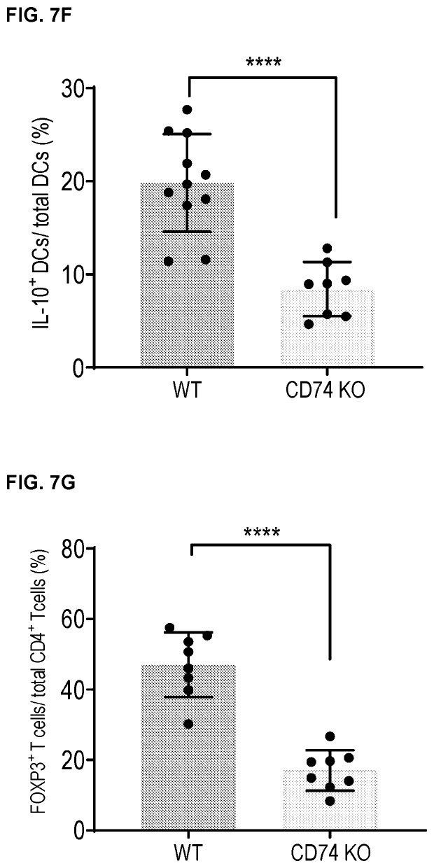

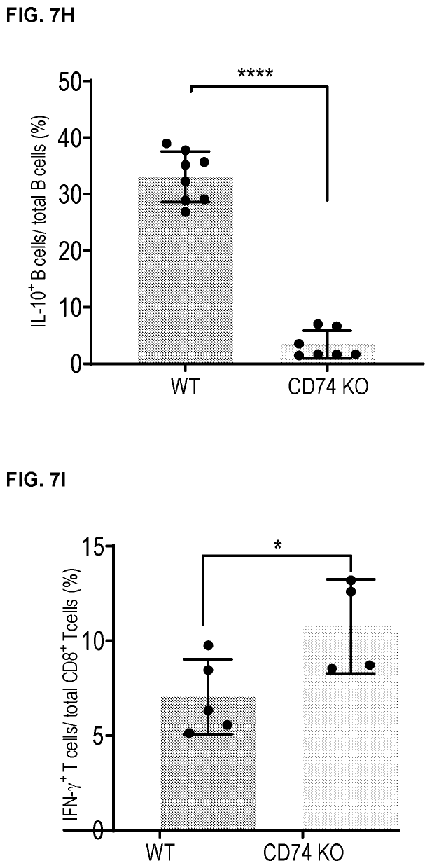

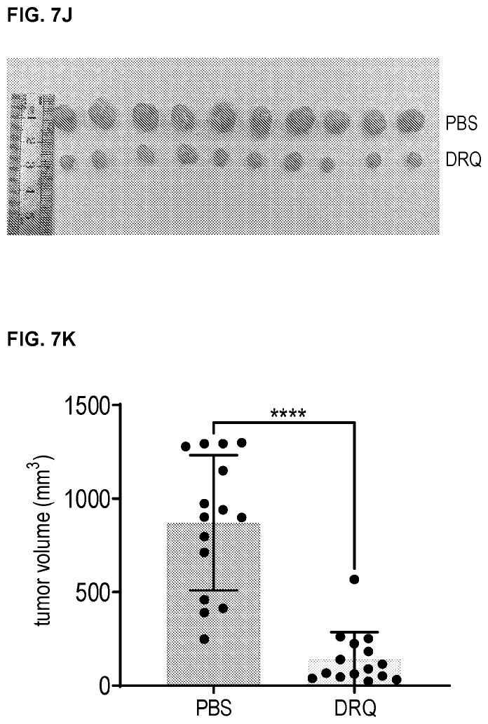

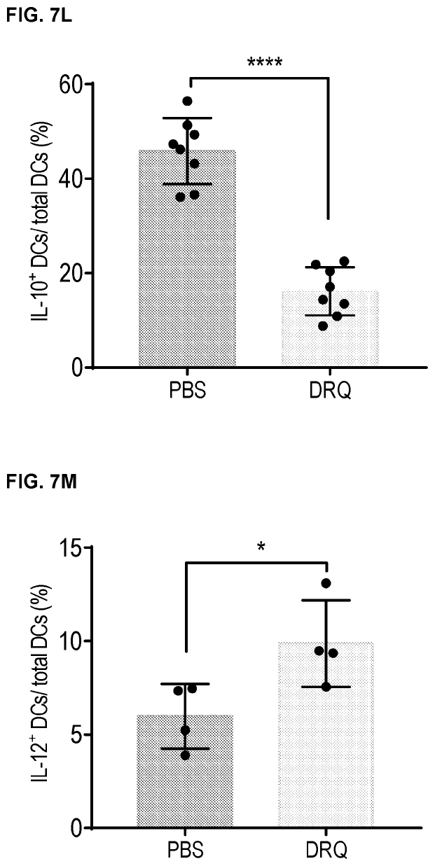

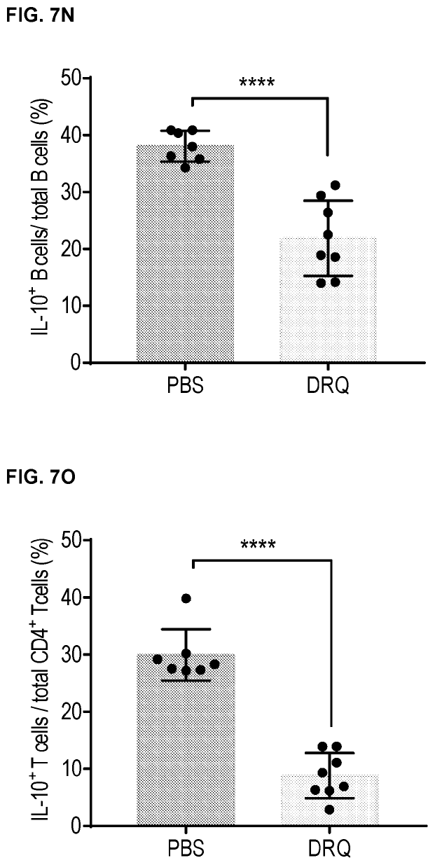

FIGS. 7A-7O illustrate that CD74 regulates tumor progression. FIGS. 7A-7I show 6 weeks old C57BL/6 and CD74^{-/-} female mice were injected with 5*10⁵ E0771 cells into each of the 4^{th} mammary pads (total of 2 mammary pads per mouse). Tumor growth was measured every 5 days in FIG. 7A. After 21 days mice were euthanized, and tumors were removed and measured in FIGS. 7B-7C; (n=34), each dot on the graph represents a tumor. FIGS. 7D-7I show total PBMCs from the tumor site were activated with (PMA (phorbol 12-myristate 13-acetate), ionomycin, monensin) PIM and then analyzed by flow cytometry. Dead cells were excluded from analysis by Zombie Live/Dead staining. DC cells were analyzed for CD45, CD11c, and CD80 expression after excluding LY6-C⁺, F4/80⁺ and CD19⁺ cells. FIG. 7D graph shows the mean and SD percentage of DCs in the tumor site (WT n=11; CD74^{-/-} n=8). In FIG. 7E, CD80 DCs were analyzed as percentage out of total DCs (WT n=11; CD74^{-/-} n=8). In FIG. 7F, Tolerogenic DCs were analyzed for IL-10 among total DCs. Graph shows the mean and SD percentage of IL-10⁺ DCs out of total DC cells (WT n=11; CD74KO n=8). FIG. 7G graph shows the mean and SD percentage of FOXP3⁺ T cells out of total CD4⁺ T cells (WT n=8; CD74KO n=8). FIG. 7H graph shows the mean and SD percentage of IL-10+B cells out of total B cells (WT n=8; CD74 KO n=7). FIG. 7I graph shows the mean and SD percentage of IFN-γ⁺ T cells out of total CD8⁺ T cells (WT n=5; CD74KO n=4). FIGS. 7J-7O show female 6 weeks old C57BL/6 mice were injected with 5*10⁵ E0771 cells into each of the 4^{th} mammary pads and injected intravenously with DRQ on days 10, 11, 12, 13, 14, after tumor onset. After 21 days, tumor sizes were measured and recorded (n=29) in FIGS. 7J-7K. In FIG. 7L, IL-10⁻ DCs were analyzed as the percentage of total DCs (for PBS n=8; DRQ n=8). In FIG. 7M, IL-12⁺ DCs were analyzed as the percentage of total DCs (for PBS n=4; DRQ n=4). FIG. 7N graph shows the mean and SD percentage of IL-10⁺ B cells out of total B cells (for PBS n=7; DRQ n=8). FIG. 7O graph shows the mean and SD percentage of Tregs out of total CD4⁺ cells (for PBS n=7; DRQ n=8). * p<0.05, ** p<0.005, *** P<0.0005, **** p<0.00005.

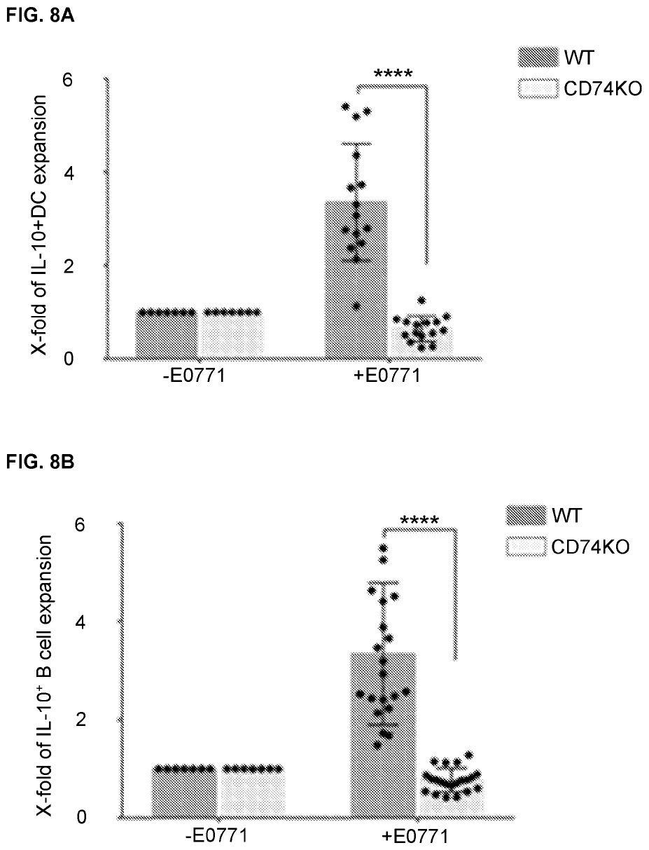

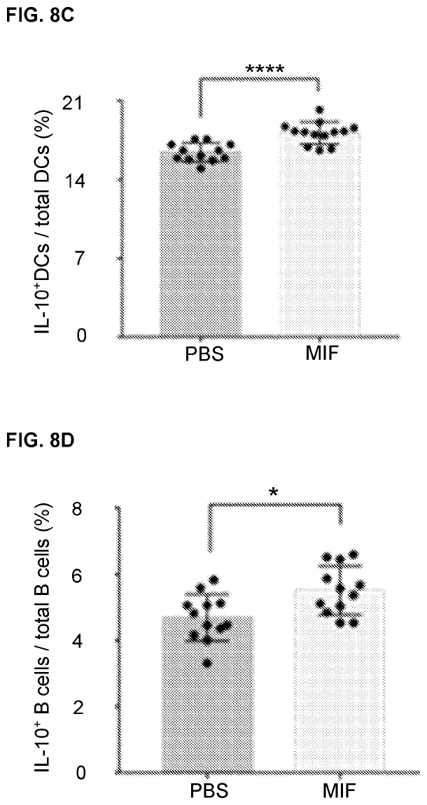

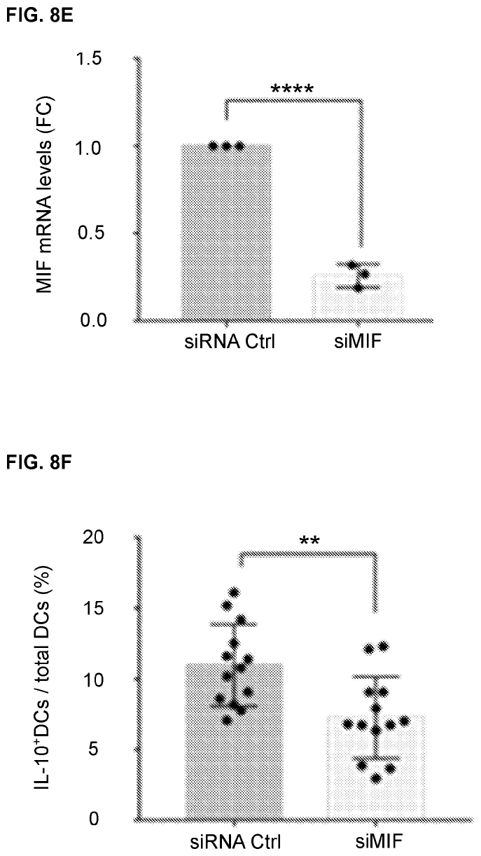

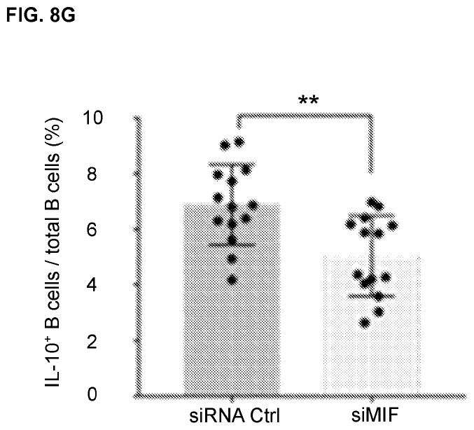

FIGS. 8A-8G illustrate that MIF-CD74 axis regulates tol-DC and Breg expansion. In FIGS. 8A-8B, B cells and DCs were isolated from spleens of naïve C57BL/6 and CD74^{-/-} mice and cultured either alone or in co-culture with E0771 cells at a 1:5 (B cells: E0771) or 1:3 (DCs: E0771) ratio. After 24 hours, B cells or DCs were collected and analyzed for CD19 and IL-10 expression or for CD11c and IL-10 expression, respectively, after excluding LY6-C⁺, F4/80⁺, and CD19⁺ cells, by flow cytometry. Cells were activated with PIM prior to FACS staining. Dead cells were excluded from analysis by Zombie Live/Dead staining. FIG. 8A shows fold change of IL10⁺ DCs expansion out of total dendritic cells, under the different culture conditions (WT n=15; CD74KO n=15), each dot represents a mouse. FIG. 8B shows fold change of Bregs expansion out of B cells, under the different culture conditions (WT n=21; CD74KO n=21). In FIGS. 8C-8D, splenic B cells and DCs were purified from IL-10 vert-x mice and cultured with E0771 cells at a 1:5 ratio, in presence of absence of mrMIF for 24 h. FIG. 8C graph shows the mean and SD percentage of IL 10⁺ DCs out of total dendritic cells (WT+PBS n=12; WT+MIF n=13). FIG. 8D graph shows the mean and SD percentage of Bregs out of B cells (WT+PBS n=12; WT+MIF n=12). In FIGS. 8E-8G, E0771 cells were transfected with siRNA MIF or siCtrl. Splenic B cells and DCs were purified from IL-10 vert-x mice and added to the transfected E0771 at a 1:5 or 1:3 ratio for 24 h. In FIG. 8E, MIF mRNA levels were analyzed by qRT-PCR. Graphs present fold change (siRNA/siCtrl) of the chosen gene (n=3). FIG. 8F graph shows the mean and SD percentage of IL10⁺ DCs out of total dendritic cells (WT+Sico E0771 n=13; WT+siMIF n=13). FIG. 8G bar graphs represent the mean and SD percentage of Bregs out of B cells (WT+Sico E0771 n=14; WT+siMIF n=14). * p<0.05, ** p<0.005, *** P<0.0005, **** p<0.00005.

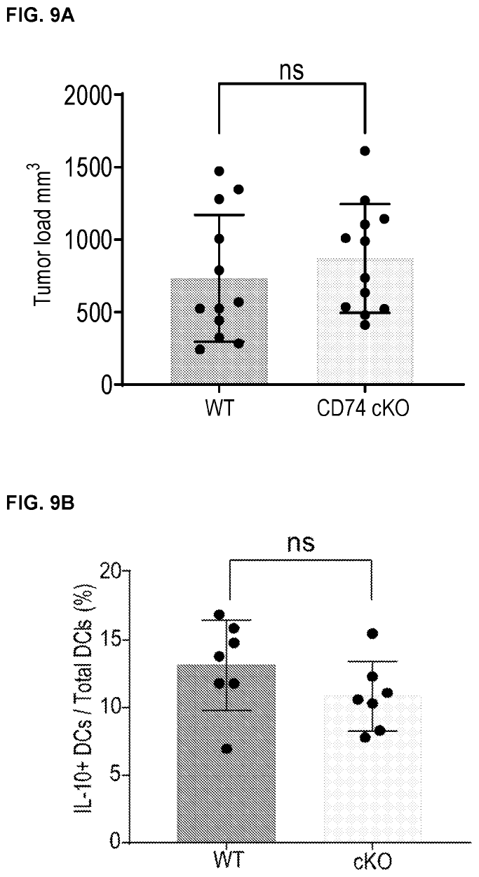

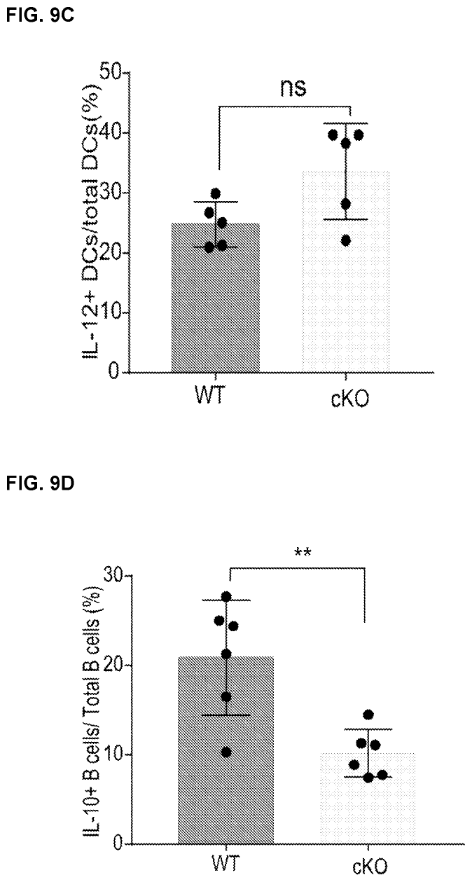

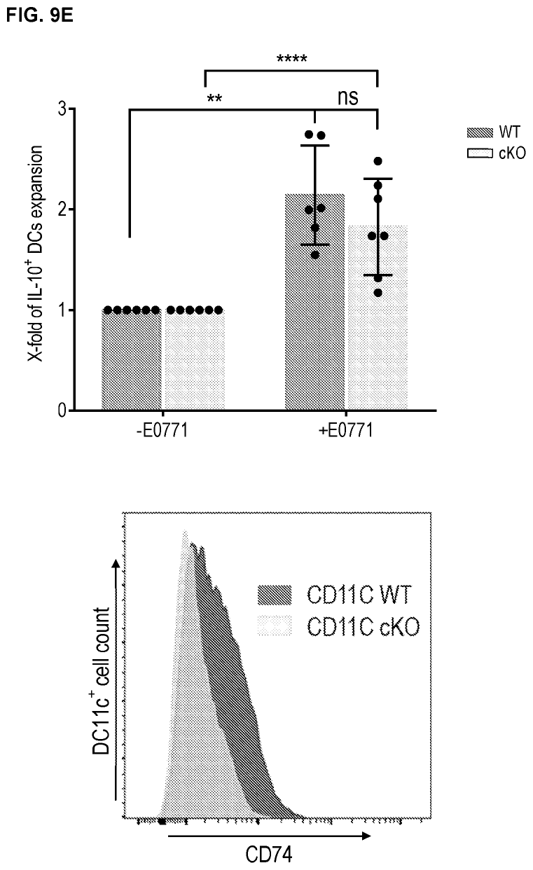

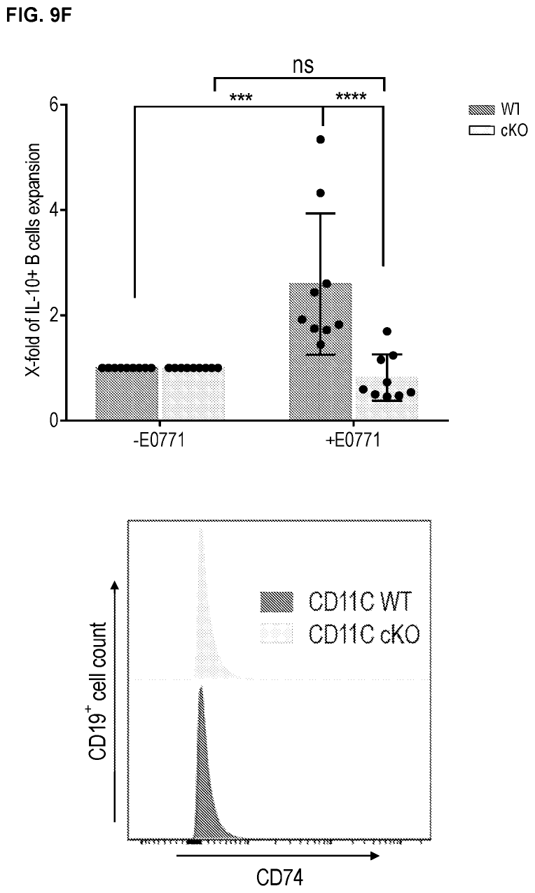

FIGS. 9A-9F illustrate that deficiency of CD74 in mature B cells does not affect tumor proliferation. FIGS. 9A-9D show female 6-weeks-old CD23-Cre×CD74flox×CD74 flox mice were injected with 5*10⁵ E0771 cells into each of the 4^{th} mammary pads. After 21 days, tumor sizes were measured and recorded (n=22) in FIG. 9A. Mice were euthanized, and tumors were harvested, processed to a single cell suspension, and total PBMCs from the tumor site were isolated. Cells were then activated with PIM and analyzed by flow cytometry. Dead cells were excluded from analysis by Zombie Live/Dead staining. DC cells were analyzed for CD45, CD11c and IL-10 expression after excluding LY6-C⁺, F4/80⁺, and CD19⁺ cells. Bregs were analyzed for CD19 and IL-10. In FIG. 9B, IL-10⁺ DCs were analyzed as the percentage of total DCs (for the WT n=8; conditional CKO n=7). FIG. 9C graph shows the mean and SD percentage of IL-12⁺ dendritic cells out of total DCs (for the WT n=5; CKO n=5). FIG. 9D graph shows the mean and SD percentage of IL-10⁺ B cells out of total CD19⁺ cells (for the WT n=6; CKO n=6). In FIGS. 9E-9F, splenic B cells and DCs were cultured with E0771 cells at a 1:5 ratio for 24 h. FIG. 9E graph shows the mean and SD percentage of IL10⁺ DCs out of total dendritic cells (WT n=5; C KO n=5). FIG. 9F graph shows the mean and SD percentage of Bregs out of B cells. (WT n=7; CKO n=7). The histograms represent the expression of CD74 in the DC and B cell populations. ns p>0.05, * p<0.05, ** P<0.005, *** p<0.0005.

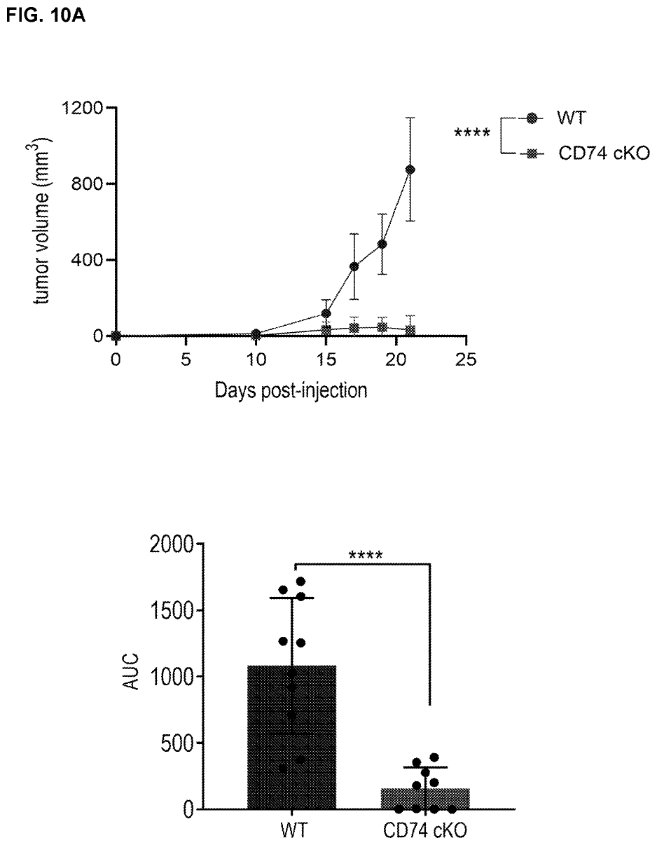

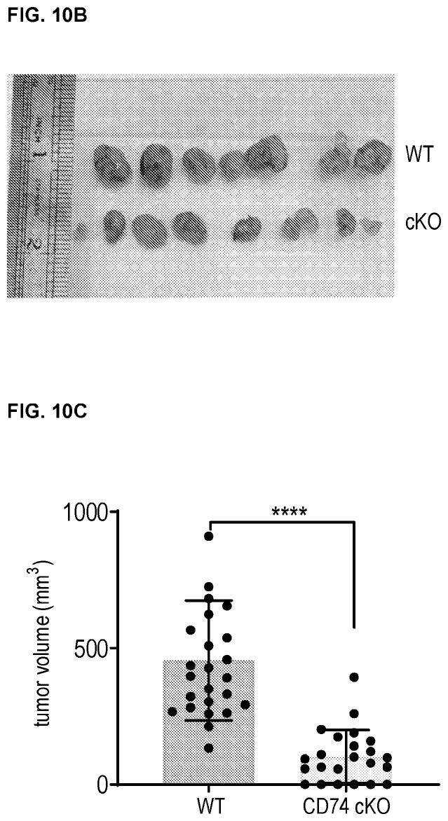

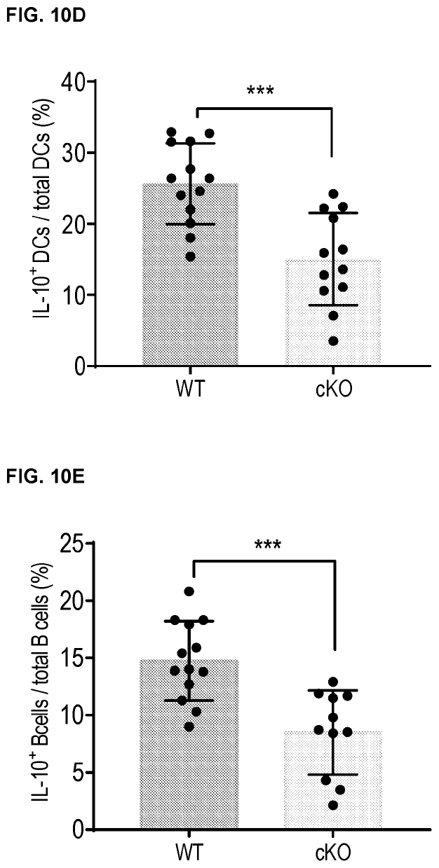

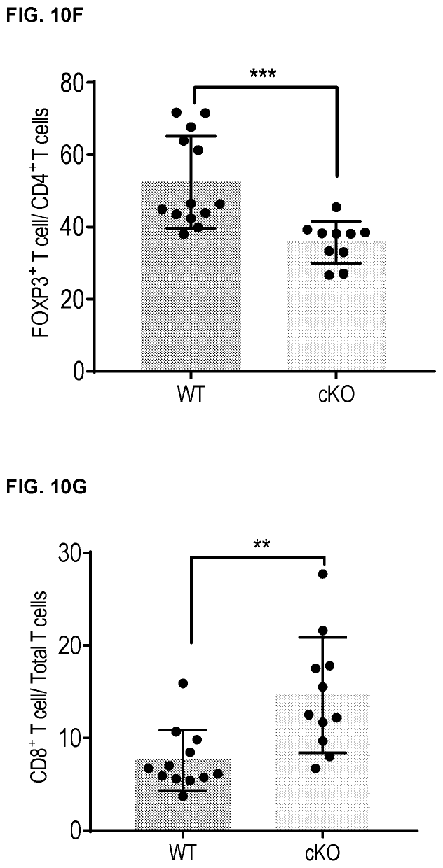

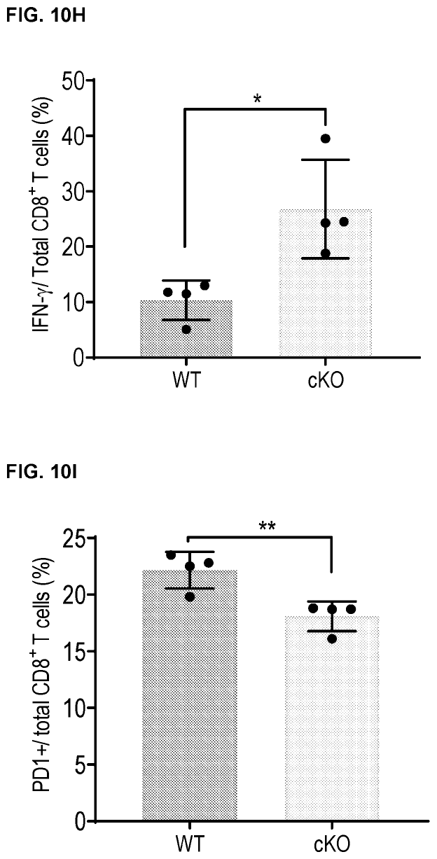

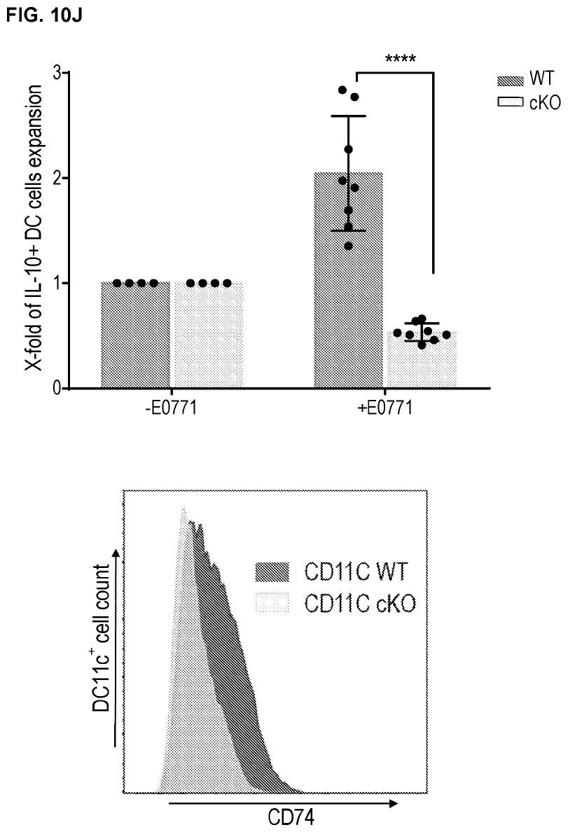

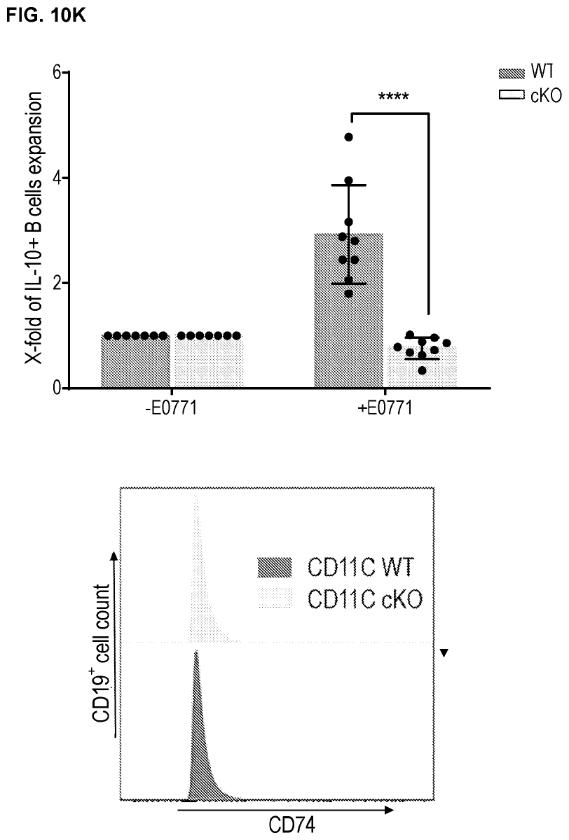

FIGS. 10A-10K illustrate that CD74 deficiency in dendritic cells reduces tumor proliferation by activation of the immune response. FIGS. 10A-10I show female 6-weeks-old CD11c-Cre×CD74flox×CD74 flox mice were injected with 5*10⁵ E0771 cells into each of the 4^{th} mammary pads. Tumor size was recorded every 5 days in FIG. 10A. After 21 days, tumor sizes were measured and recorded (n=44) in FIGS. 10B-10C; each dot represents a tumor. In FIG. 10D, IL-10⁺ DCs were analyzed as a percentage of total DCs (WT n=13; conditional CKO n=12). FIG. 10E graph shows the mean and SD percentage of IL-10⁺ B cells out of total B cells (WT n=12; CKO n=11). FIG. 10F graph shows the mean and SD percentage of Tregs out of total CD4⁺ cells (WT n=13; CKO n=10). FIG. 10G graph shows the mean and SD percentage of the tumor-infiltrating CD8⁺ T-cells out of total CD3⁺ T cells (WT n=13; CKO n=12). FIG. 10H graph shows the mean and SD percentage of the IFN-γ releasing T-cells out of total CD8⁺ T-cells (WT n=4; CKO n=4). FIG. 10I graph shows the mean and SD percentage of PD1⁺ T-cells out of total CD8⁺ T-cells (WT n=4; C KO n=4). In FIGS. 10J-10K, splenic B cells and DCs were cultured with E0771 cells at a 1:5 ratio. FIG. 10J graph shows the mean and SD percentage of IL10⁺ DCs out of total dendritic cells. (WT n=8; conditional CD74 KO n=8). FIG. 10K graph shows the mean and SD percentage of Bregs out of B cells (WT n=8; conditional CD74-n=8). The histograms represent the expression of CD74 in the DC and B cells populations. ns p>0.05, * p<0.05, *** P<0.0005, **** p<0.00005.

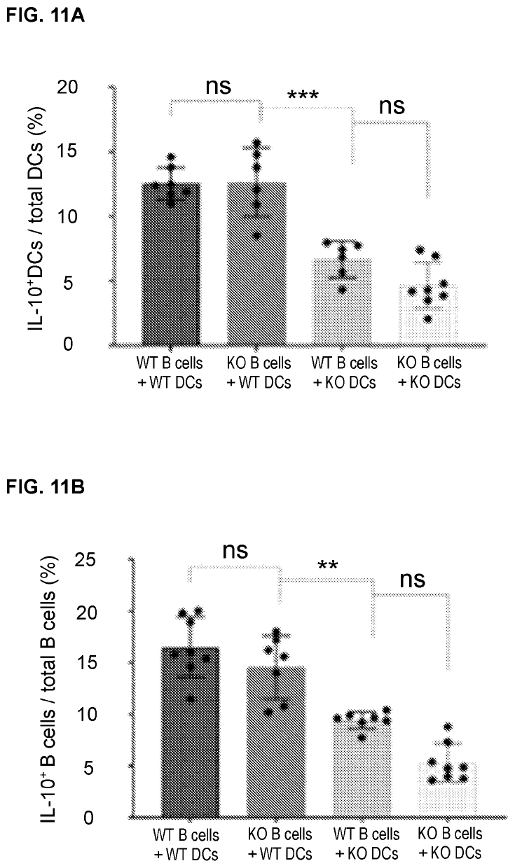

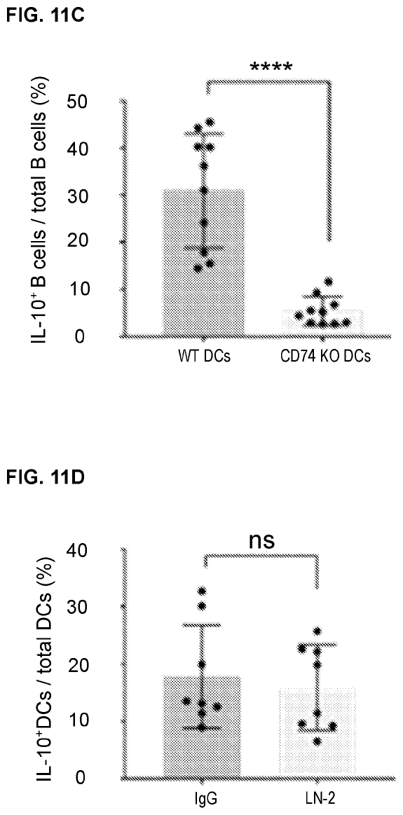

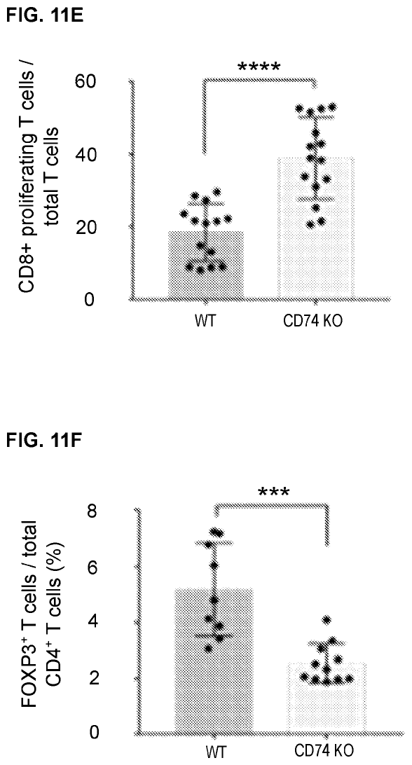

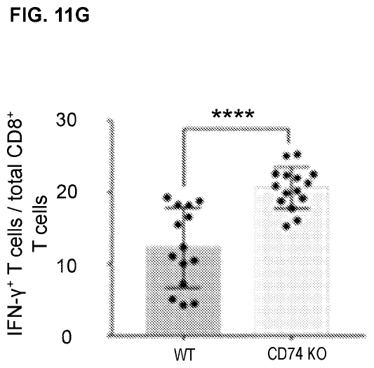

FIGS. 11A-11G illustrate that CD74 mediates the crosstalk between DC and B cells enhancing immunosuppression in the tumor ME. In FIGS. 11A-11B, B cells and DCs were isolated from spleens of naïve C57BL/6 and CD74-mice and cultured at a 1:1 ratio; E0771 cells were added at a 1:5 ratio. After 24 hours, B cells or DCs were collected and analyzed by flow-cytometry. PIM activation was conducted prior to FACS staining. Dead cells were excluded from analysis by Zombie Live/Dead staining. FIG. 11A graph shows the mean and SD percentage of IL 10⁺ dendritic cells out of total dendritic cells, under the different culture conditions (WT DCs+WT B cells n=8; CD75T4 KO DCs+WT B cells n=8; WT DCs+CD74 KO B cells n=8; CD74^{-/-} DCs+CD74 KO B cells n=8). FIG. 11B graph shows the mean and SD percentage of IL 10⁺ B cells out of total B cells, under the different culture conditions (WT DCs+WT B cells n=8; CD74 KO DCs+WT B cells n=8; WT DCs+CD74 KO B cells n=8; CD74 KO DCs+CD74 KO B cells n=8). In FIG. 11C, splenic DCs were purified from CD11c-Cre×CD74flox×CD74 flox mice and activated with E0771 for 24 h. Naive splenic B cells were purified from C57BL/6 mice and activated with the WT or CD74^{-/-} DC for 24 h with fresh medium. Bar graphs depict the mean and SD percentage of IL 10⁺ B cells out of total B cells (B cells+WT DCs CM n=10; B cells+CD74 cKO DCs CM n=10). In FIG. 11D, splenic B cells were purified from C57BL/6 mice, activated with the E0771, and incubated with an anti-CD74 blocking antibody (LN-2) or isotype control antibody for 24 h. Naive splenic DCs were purified from C57BL/6 mice and cultured together with the treated B cells with a fresh medium. Bar graph depicts the mean and SD percentage of IL10⁺ dendritic cells out of total DCs (DCs+B cells treated with IGg n=8; DCs+B cells treated with LN-2 n=8). In FIGS. 11E-11G, splenic DCs were purified using magnetic beads from C57BL/6 and CD74^{-/-} mice and co-cultured with E0771 at a 1:3 ratio for 24 h. Naive Splenic CD3⁺ T cells were purified from C57BL/6 mice and stained with the Cell Proliferation Dye (CPD) to determine their ability to proliferate. Following co-culture with E0771, DCs were cultured with T cells at a 1:1 ratio in the presence of IL-2 in the media for 72h. T cells were analyzed for CD8, FOXP3, and INF-γ. Dead cells were excluded from analysis by Zombie Live/Dead staining. FIG. 11E graph shows the mean and SD percentage of proliferating CD8⁺ T cells under the different culture conditions (WT n=14; CD74 KO n=15). FIG. 11F graph shows the mean and SD percentage of FOXP3⁺ CD4⁺ T cells under the indicated culture conditions (WT n=9; CD74^{-/-} n=11). FIG. 11G graph depicts the percentage of IFN-γ⁺ CD8⁺ T cells (WT n=14; CD74 KO n=15).

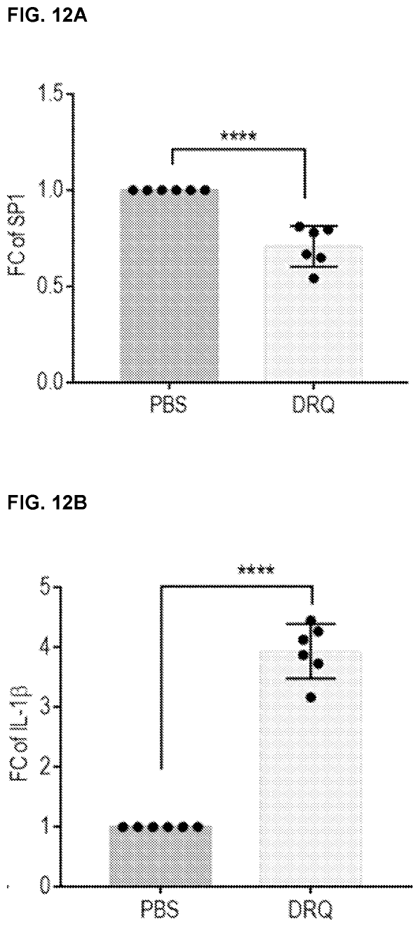

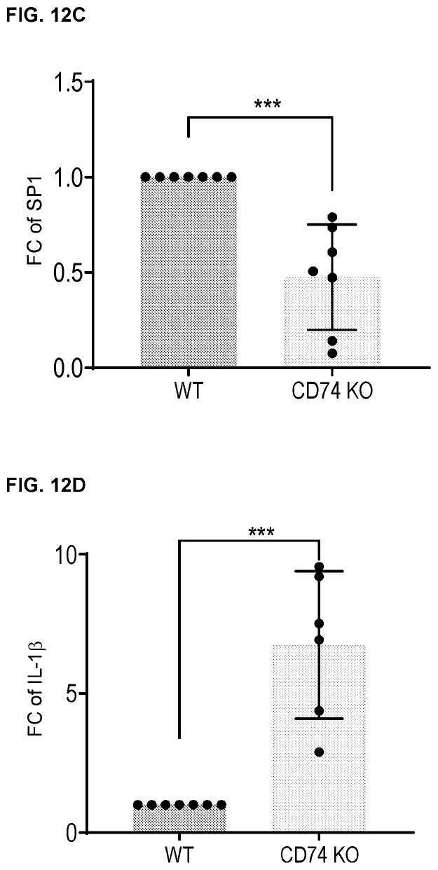

FIGS. 12A-12D illustrate that CD74 deficiency in dendritic cells induces pro-inflammatory pathways boosting the anti-tumor immune response. Female 6 weeks old C57BL/6 mice were injected with 5*10⁵ E0771 cells into each of the 4^{th} mammary pads. DRQ was injected intravenously on days 10,11, 12, 13, and 14, after tumor onset. After 21 days, tumor sizes were measured, and mice were euthanized. Tumors were processed into a single-cell suspension, and DCs were sorted from the tumor microenvironment of mice treated either with PBS or DRQ. Four replicates were analyzed for each group. Based on the heatmap of diseases, disorders and biological functions generated for all differentially expressed genes from the RNA-seq data of DRQ vs PBS, it was indicated that genes involved in cancer progression were downregulated in the DRQ-treated samples, while genes related to immune trafficking, cellular movement and inflammatory response were upregulated. In FIGS. 12A-12B, mRNA levels of SP1 and IL1β, were validated by qRT-PCR. Graph presents fold change of selected genes in the PBS-treated DCs versus DRQ treated DCs (n=6). FIGS. 12C-12D show fold change of SP1 and IL1β in WT and CD74 KO mice (n=6). *** p<0.0005 **** p<0.00005.

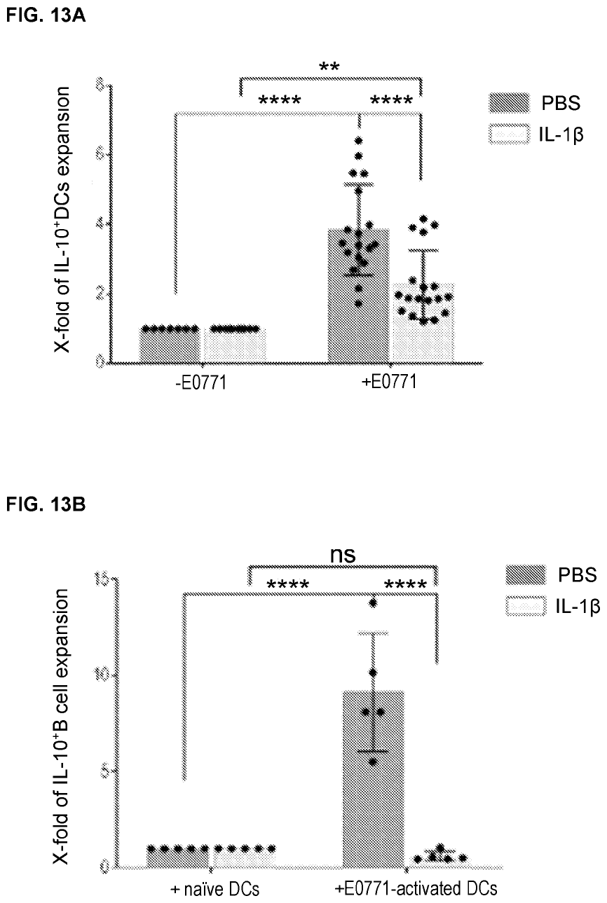

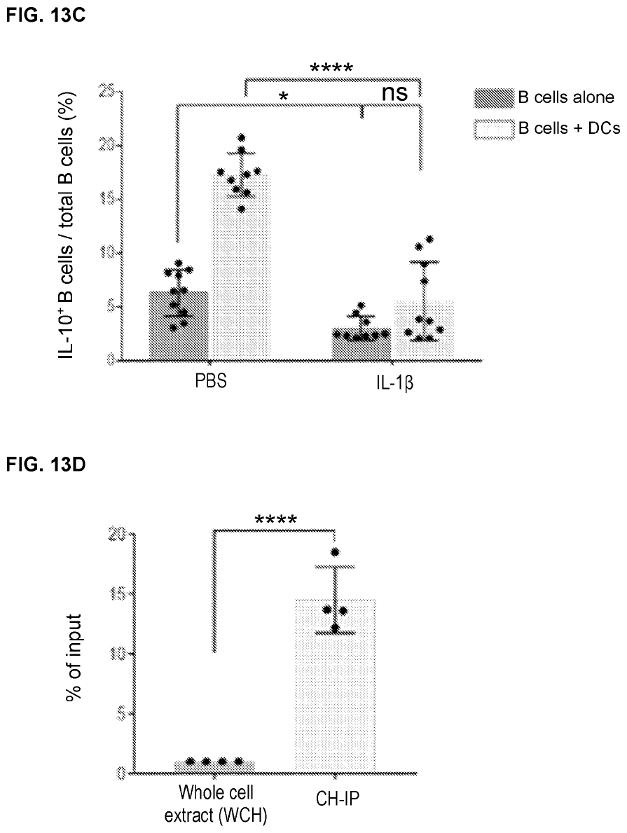

FIGS. 13A-13D illustrate that CD74-ICD binds IL-1β promotor in DC, promoting their tolerogenic phenotype. In FIGS. 13A-13C, splenic dendritic cells were isolated from vert-x mice and cultured in presence of the E0771 at a 1:3 ratio. IL-1β agonist or vehicle was added to the cells for 48h. FIG. 13A graph shows the mean and SD percentage of IL10⁺ dendritic cells out of total DCs (DCs+vehicle=18; DCs+IL1β n=20). Each dot represents a mouse. In FIG. 13B, splenic DCs were purified from C57BL/6 mice and activated with the E0771 for 24 h in presence of IL-1β agonist or PBS control. Naive splenic B cells were purified from C57BL/6 mice and added to the DCs for 24 h with a fresh medium. Bar graphs depict the mean and SD percentage of IL10⁺ B cells out of total B cells and IL10⁺ DCs out of total dendritic cells (B cells+DCs treated with PBS, n=5; B cells+DCs treated with IL-1β, n=5). In FIG. 13C, splenic B cells were purified from C57BL/6 mice and activated with E0771 cells either alone or with purified DCs for 24 h, in the presence of IL-1β or vehicle. Bar graphs depict the mean and SD percentage of IL10⁺ B cells out of total B cells (B cells treated with PBS n=10; B cells treated with IL-1β n=10; B cells+DCs treated with PBS n=10; B cells+DCs treated with IL-1β n=10). FIG. 13D shows female 6-weeks-old C57BL/6 mice were injected with 5*10⁵ E0771 cells into each of the 4^{th} mammary pads. After 21 days, mice were euthanized. Tumors were processed into a single-cell suspension and DCs were sorted from the tumor microenvironment. ChIP analysis was performed. The binding of CD74-ICD to the promotor area of the IL-1β gene was determined by qPCR. The graph represents the percentage of enrichment of the input (the amount of DNA pulled down by using the antibody of interest in the ChIP reaction, relative to the amount of starting material-input sample) (n=4). ns p>0.05, * p<0.05, **** p<0.00005.

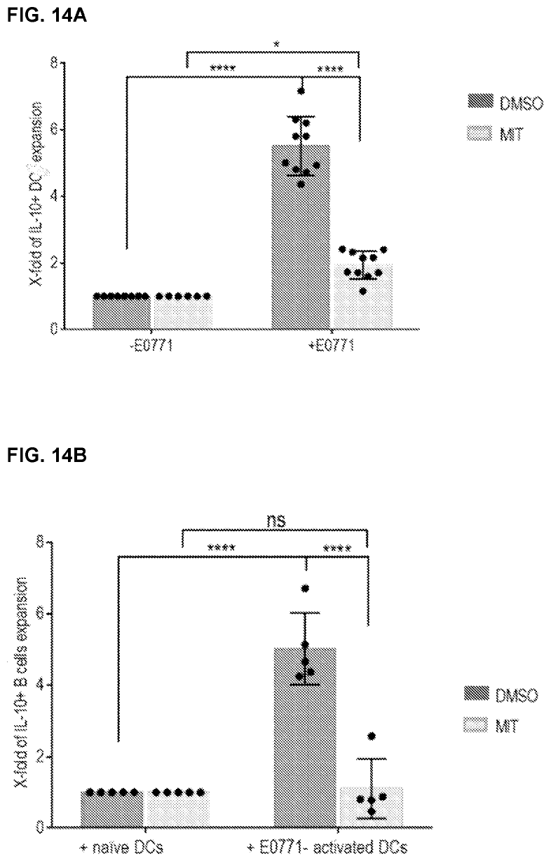

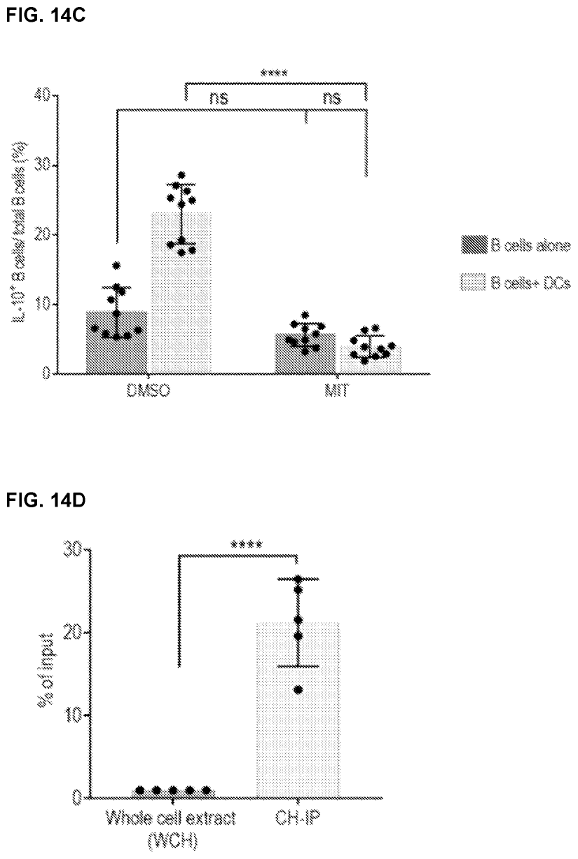

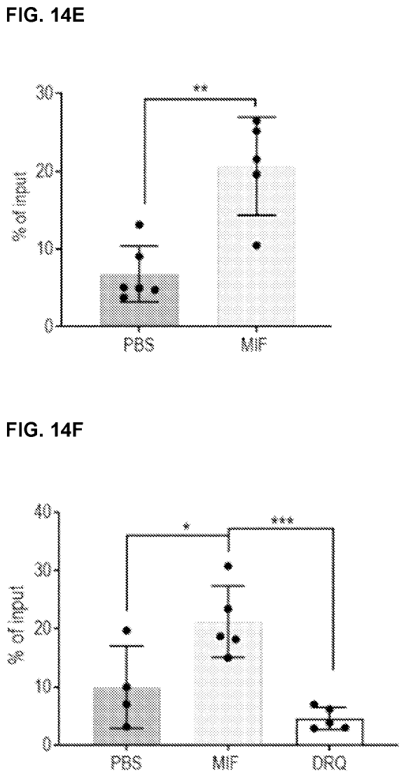

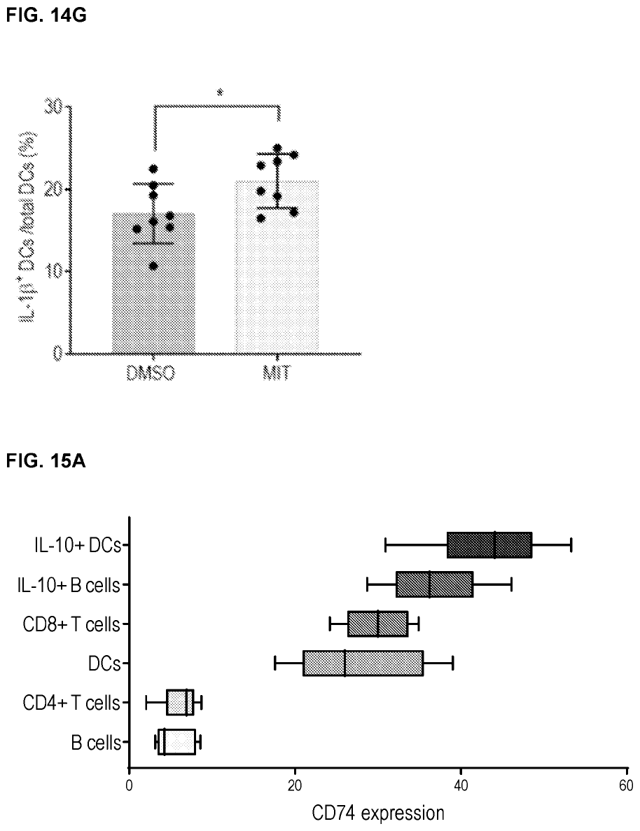

FIGS. 14A-14G illustrate that SP1 binds the IL-1β promotor on DC via the MIF-CD74 axis, inducing their tolerogenic phenotype. In FIGS. 14A-14C, splenic dendritic cells were isolated from vert-x mice and cultured in presence of E0771 cells at a 1:3 ratio. SP1 blocker (MIT) or DMSO were added to the cells for 48h. FIG. 14A shows fold change of the expansion of IL10⁺ dendritic cells out of total DCs (DCs+DMSO=10; DCs+MIT n=10). Each dot represents a mouse. In FIG. 14B, splenic DCs were purified from C57BL/6 mice and activated with the E0771 for 24 h in the presence of DMSO or MIT. Naive splenic B cells were purified from C57BL/6 mice and added to the DCs for 24 h after replacing the medium. Bar graphs depict the fold change of IL10⁺ B cells out of total B cells and IL10⁺ DCs out of total dendritic cells (B cells treated with DMSO n=5; B cells treated with mithramycin n=5). In FIG. 14C, splenic B cells were purified from C57BL/6 mice and activated with either E0771 cells alone or with purified DCs for 24 h in presence of MIT or DMSO. Bar graphs depict the mean and SD percentage of IL 10⁺ B cells out of total B cells (B cells treated with DMSO n=10; B cells treated with MIT n=10, B cells+DCs treated with DMSO n=10; B cells+DCs treated with MIT n=10). FIGS. 14D-14E show female 6-week-old C57BL/6 mice were injected with 5*10⁵ E0771 cells into each of the 4^{th} mammary pads. After 21 days, tumor sizes were measured, and mice euthanized. Tumors were processed into single cell suspension and DCs were sorted from the tumor microenvironment. Sorted DCs were activated for 1 h with either mrMIF or vehicle, and a chip-qPCR for the SP1 promotor was performed. FIG. 14D graph represents the percentage of enrichment of the input (the amount of DNA pulled down by CD74 antibody in the ChIP reaction, relative to the amount of starting material-input sample) (n=5). FIG. 14E graph shows binding of CD74-ICD in DCs activated with mrMIF (n=5). FIG. 14F shows female 6-weeks-old C57BL/6 mice were injected with 5*10⁵ E0771 cells into each of the 4th mammary pads. After 21 days, tumor sizes were measured, and mice euthanized.

Tumors were processed into single cell suspension and DCs were sorted from the tumor microenvironment. Sorted DCs were activated for 1 h with either mrMIF, DRQ or vehicle, and a ChIP-qPCR for IL-1β promoter was performed. The graph represents the percentage of enrichment of the input (the amount of DNA pulled down by using SP1 antibody in the ChIP reaction, relative to the amount of starting material-input sample) in DCs activated with mrMIF, DRQ or vehicle (n=14). In FIG. 14G, DCs were purified from C57BL/6 mice and seeded together with E077 in the presence or absence of MIT. The graph represents the mean and SD percentage of IL-1β⁺ DCs out of total DCs (DCs treated with DMSO=8, DCs treated with MIT=8). p>0.05, * p<0.05, ** P<0.005, *** P<0.0005, **** p<0.00005.

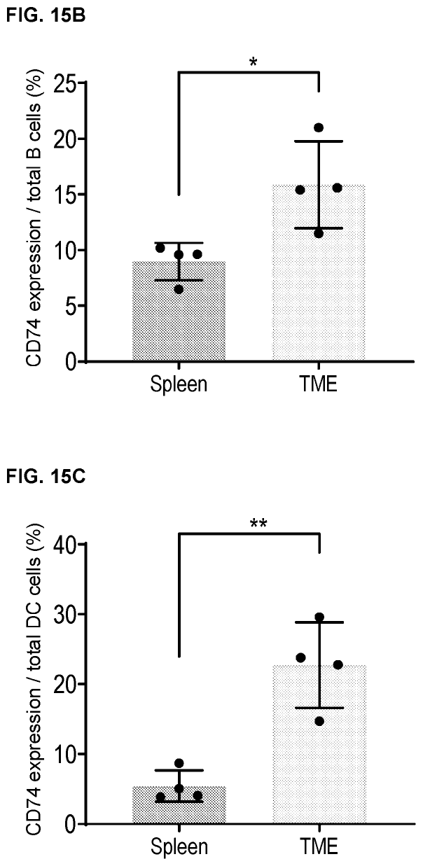

FIGS. 15A-15C illustrate that CD74 expression is upregulated in tolerogenic DCs and B cells. 6 weeks old C57BL/6 female mice were injected with 5*105 E0771 cells into each of the 4th mammary pads (total of two mammary pads per mouse). Mice were euthanized, and total PBMCs from the tumor site and spleen were activated with PIM and then analyzed by flow cytometry. Dead cells were excluded from analysis by Zombie Live/Dead staining. FIG. 15A shows box plot analysis depicting the relative expression level of CD74 in several immune cell populations. FIG. 15B graph shows the mean and SD percentage of CD74 expression on B cells in the spleen and in the TME (B cells in the spleen n=4; B cells in the TME n=4). FIG. 15C graph shows the mean and SD percentage of CD74 expression on dendritic cells in the spleen and in the TME (dendritic cells in the spleen n=4; dendritic cells in the TME n=4). * p<0.05, ** p<0.005.

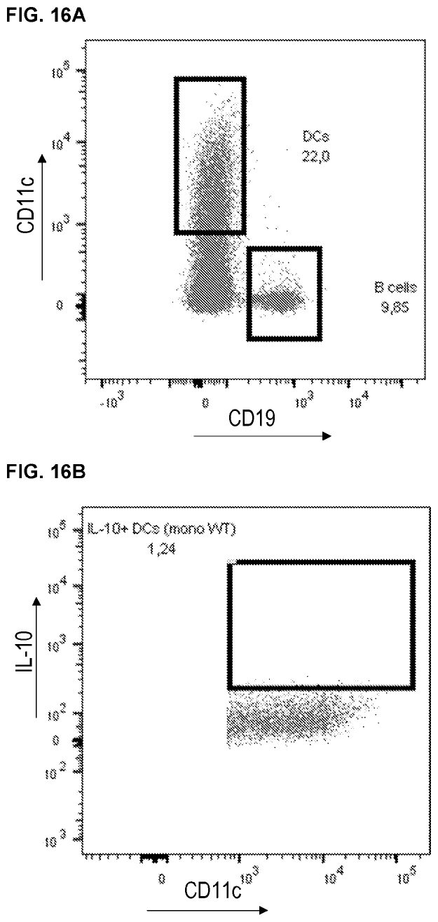

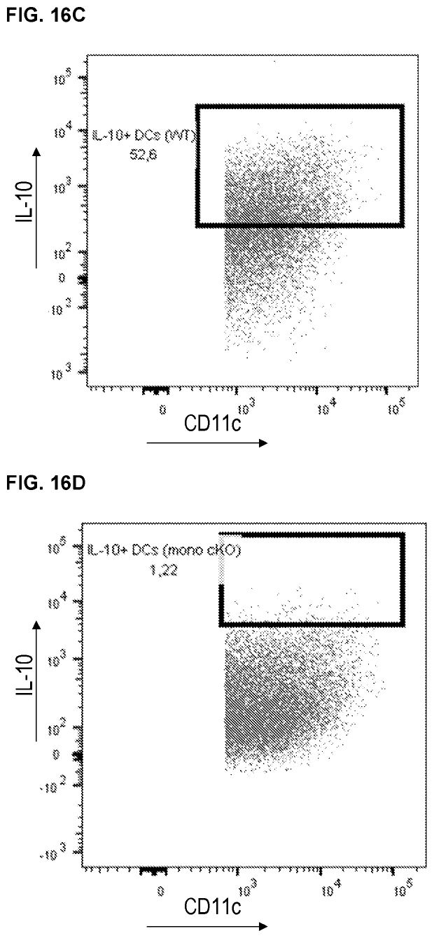

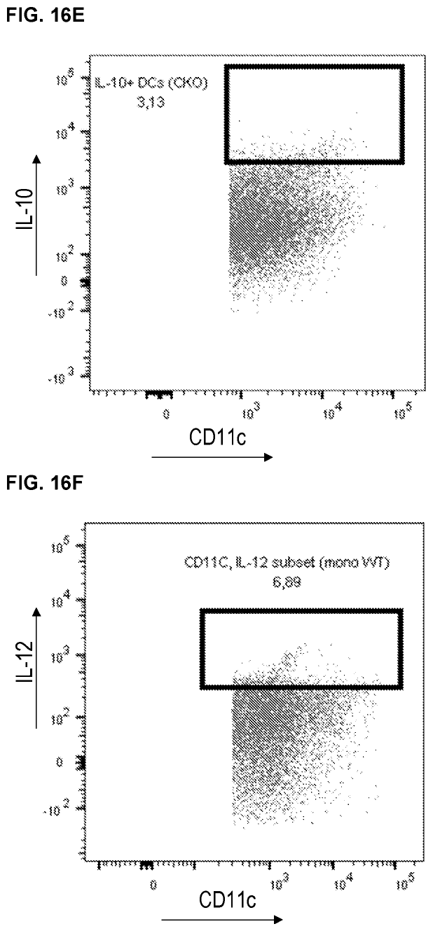

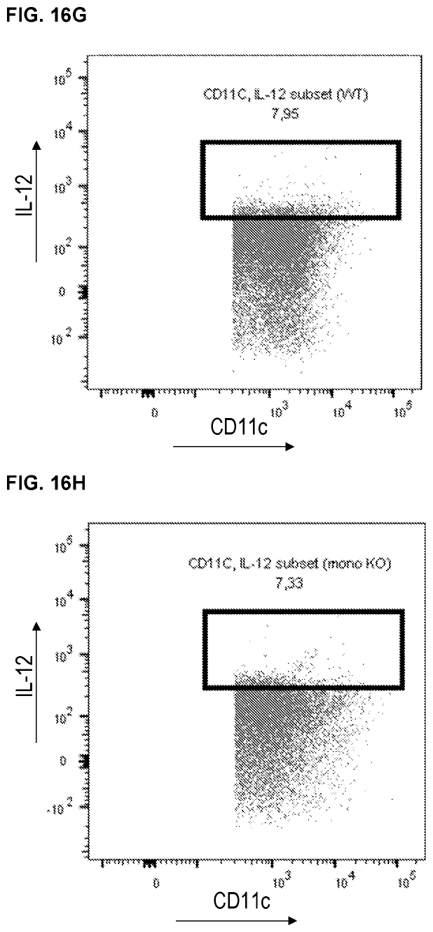

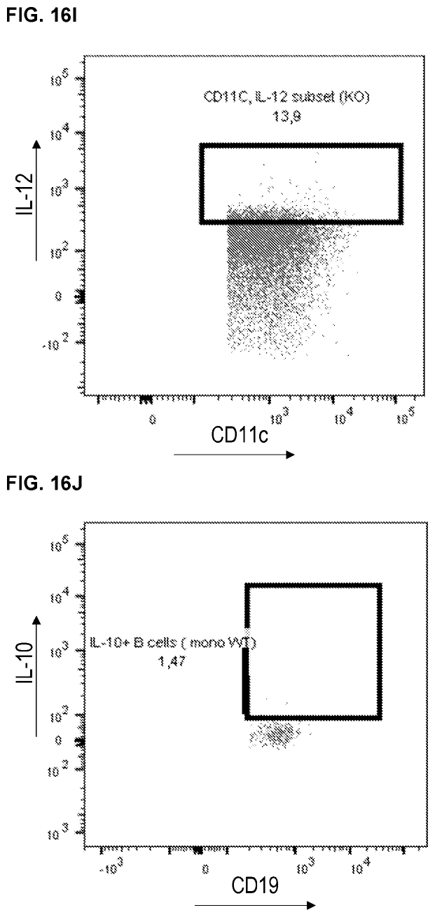

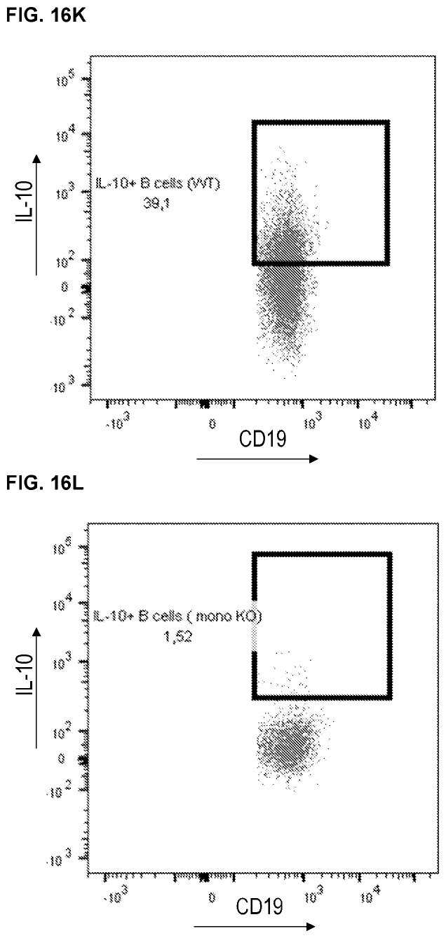

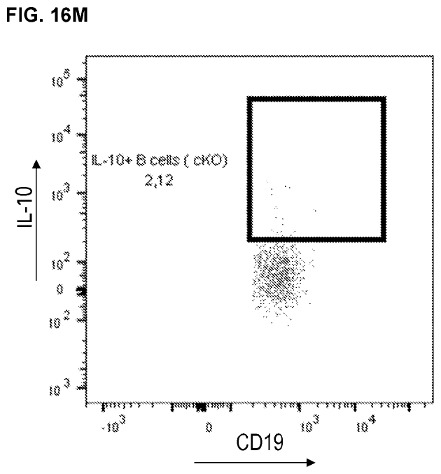

FIGS. 16A-16M illustrate a gating strategy for IL-10+ DCs and B cells and for IL-12+ DCs. PBMCs from the tumor site were activated with PIM and then analyzed by flow cytometry. Dead cells were excluded from analysis by Zombie Live/Dead staining. In FIG. 16A, DC cells were analyzed for CD11c expression after excluding LY6-C+, F4/80+ and CD19. B cells were analyzed for CD19 after excluding LY6-C+, F4/80+ and CD11c. In FIGS. 16B-16E, IL-10+ expression on DCs was measured by comparing the non-activated for either WT and CD74−/− samples with the ones activated with PIM. In FIGS. 16F-16I, IL-12+ expression on DCs was measured by comparing the non-activated for either WT and CD74−/− samples with the ones activated with PIM. In FIGS. 16J-16M, IL-10+ expression on B cells was measured by comparing the nonactivated for either WT and CD74−/− samples with the ones activated with PIM.

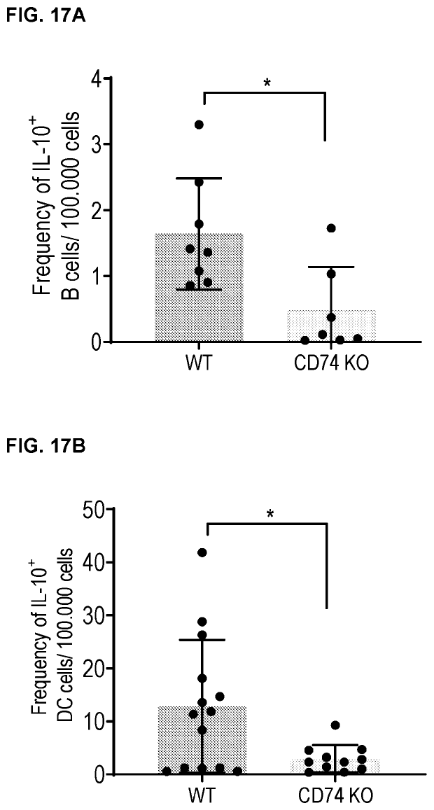

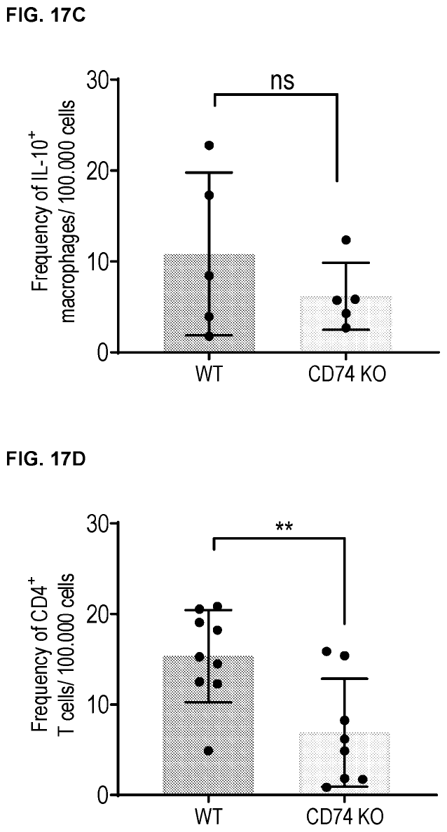

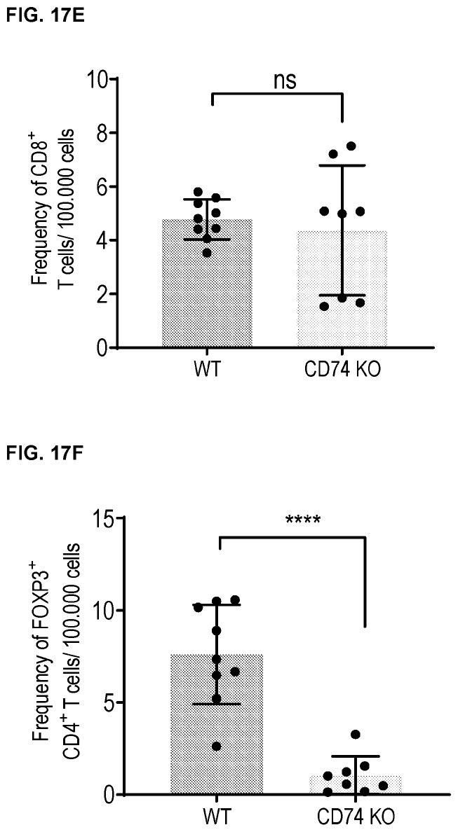

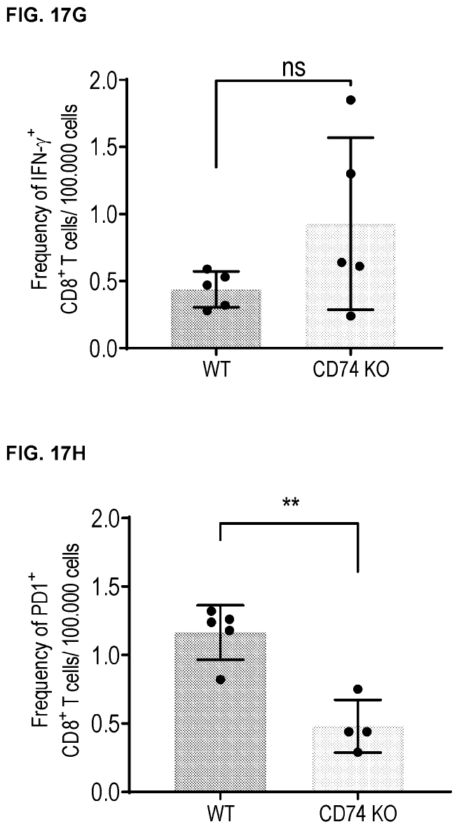

FIGS. 17A-17I illustrate that CD74 regulates the accumulation of tolerogenic immune cells in the TME. 6 weeks old C57BL/6 and CD74−/− female mice were injected with 5*105 E0771 cells into each of the 4th mammary pads (total of two mammary pads per mouse). FIG. 17A shows frequency of IL-10+ B cells out of total B cells (WT n=8; CD74−/− n=7). In FIG. 17B, DC cells were analyzed for CD45, CD11c, and IL-10 expression after excluding LY6-C+, F4/80+ and CD19+ cells. Graph shows the frequency of IL-10+ DCs in the tumor site (WT n=14; CD74−/− n=11). FIG. 17C shows frequency of IL-10+ macrophages after excluding monocytes and DCs (WT n=5; CD74−/− n=5). FIG. 17D shows frequency of CD4+ T-cells (WT n=9; CD74−/− n=8). FIG. 17E shows frequency of CD8+ T-cells (WT n=9; CD74−/− n=8). FIG. 17F shows frequency of FOXP3+ T cells out of total CD4+ T cells (WT n=9; CD74−/− n=8). FIG. 17G shows frequency of IFN-γ+ T cells out of total CD8+ T cells (WT n=5; CD74−/−n=4). FIG. 17H shows frequency of PD1+ T cells out of total CD8+ T cells (WT n=5; CD74−/− n=4). FIG. 17I shows frequency of CD62L+ T cells out of total CD8+ T cells (WT n=5; CD74−/− n=5). ns p>0.05, * p<0.05, ** p<0.005, **** p<0.00005.

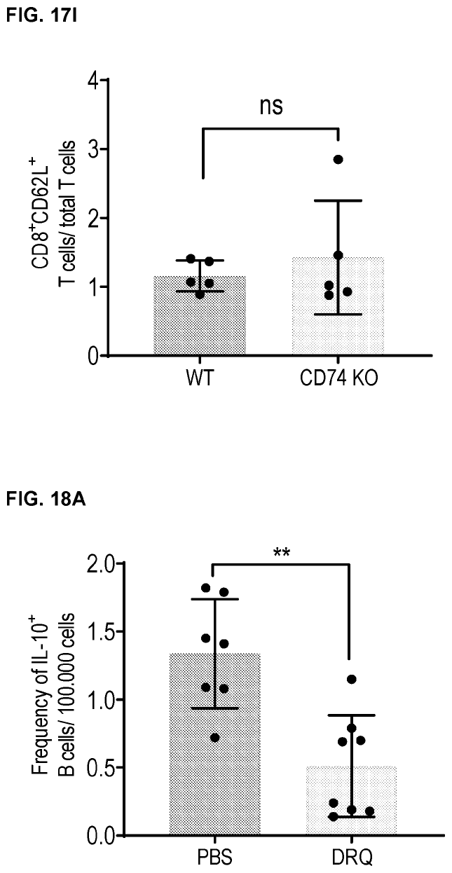

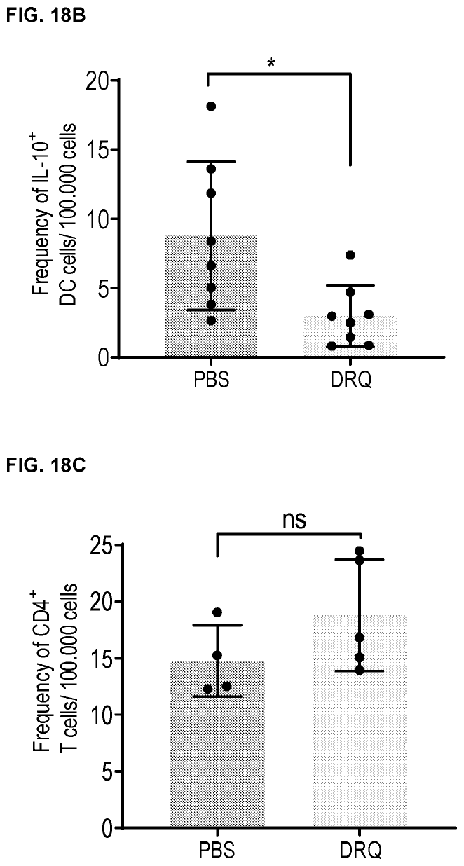

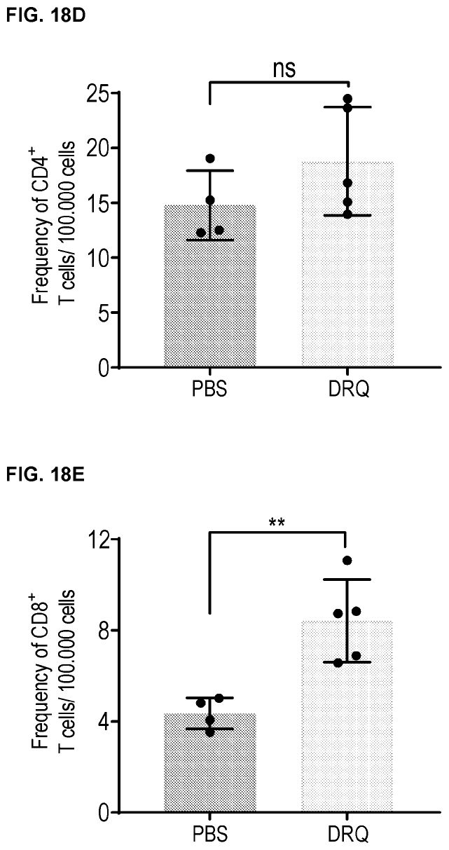

FIGS. 18A-18E illustrate that the CD74 blocker DRQ restores the immunogenicity of the TME. 6 weeks old C57BL/6 female mice were injected with 5*105 E0771 cells into each of the 4th mammary pads (total of two mammary pads per mouse). On days 10, 11, 12, 13, 14, after tumor implantation, DRQ was intravenously injected. FIG. 18A shows frequency of IL-10+ B cells out of total B cells (PBS n=7; DRQ n=8). In FIG. 18B, DC cells were analyzed for CD45, CD11c, and IL-10 expression after excluding LY6-C+, F4/80+ and CD19+ cells. Graph shows the frequency of IL-10+ DCs in the tumor site (PBS n=8; DRQ n=8). FIG. 18C shows frequency of CD4+ T-cells (PBS n=4; DRQ n=5). FIG. 18D shows frequency of FOXP3+ T cells out of total CD4+ T cells (PBS n=4; DRQ n=5). FIG. 18E shows frequency of CD8+ T-cells (PBS n=4; DRQ n=5). ns p>0.05, * p<0.05, ** p<0.005.

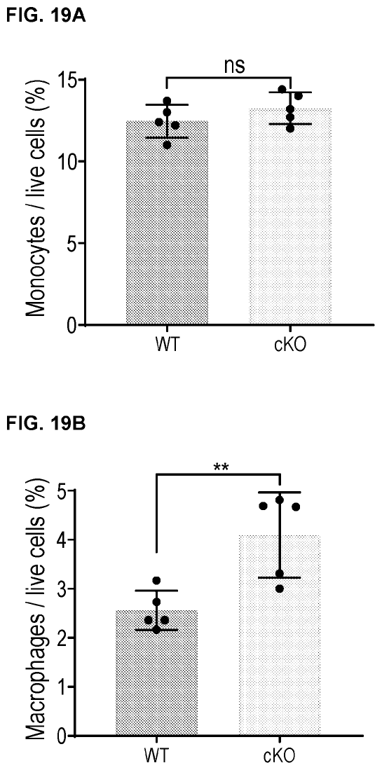

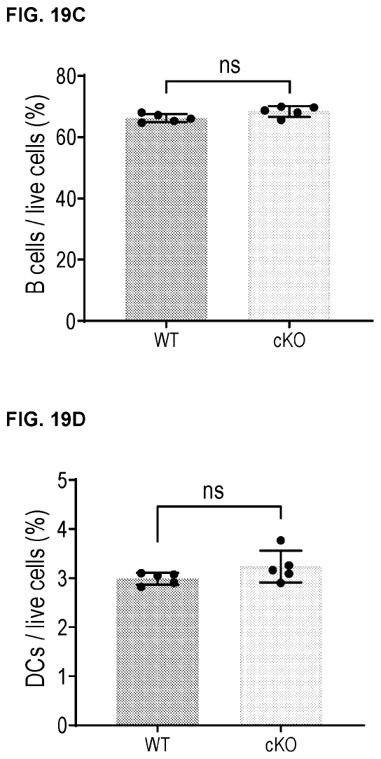

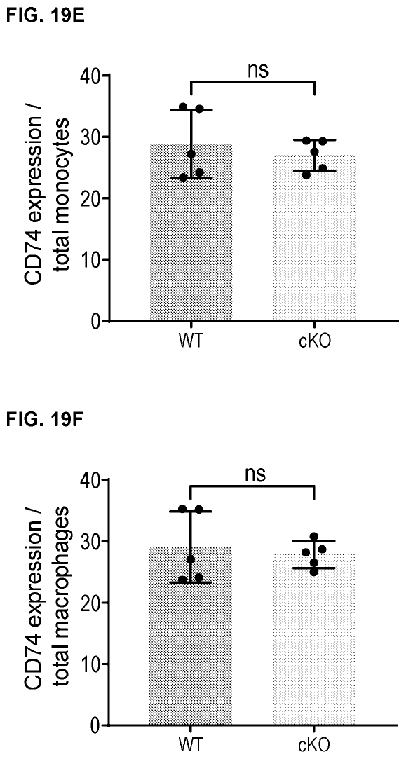

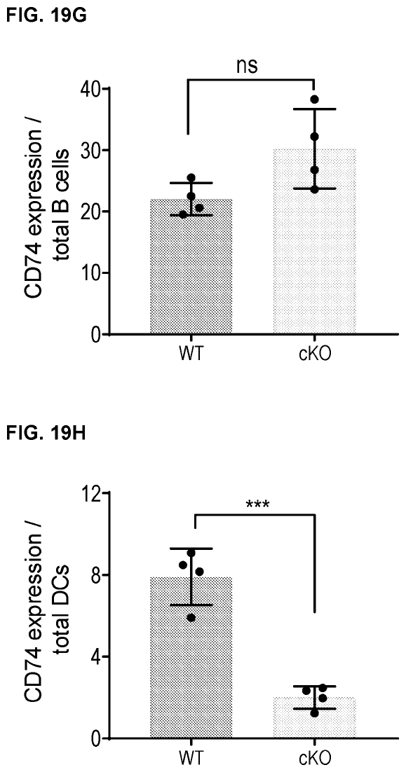

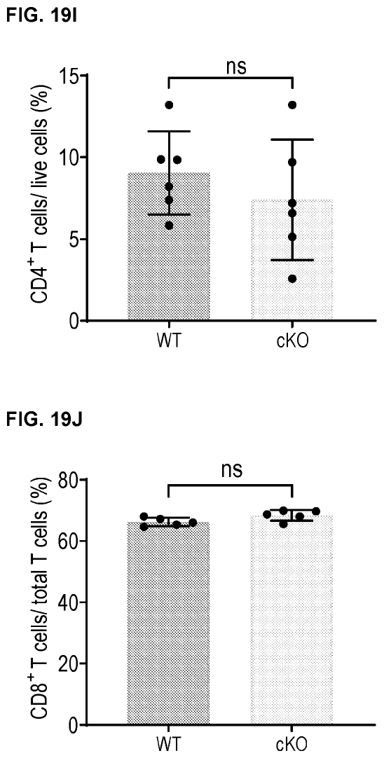

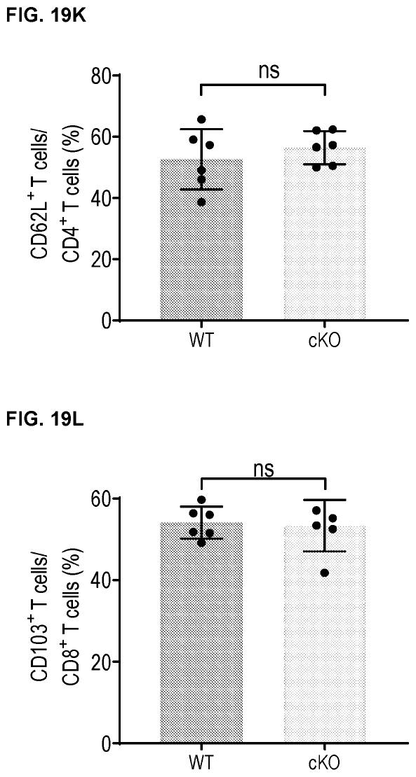

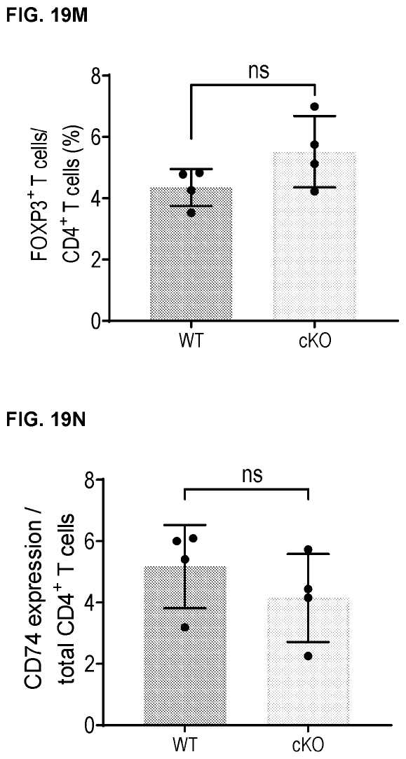

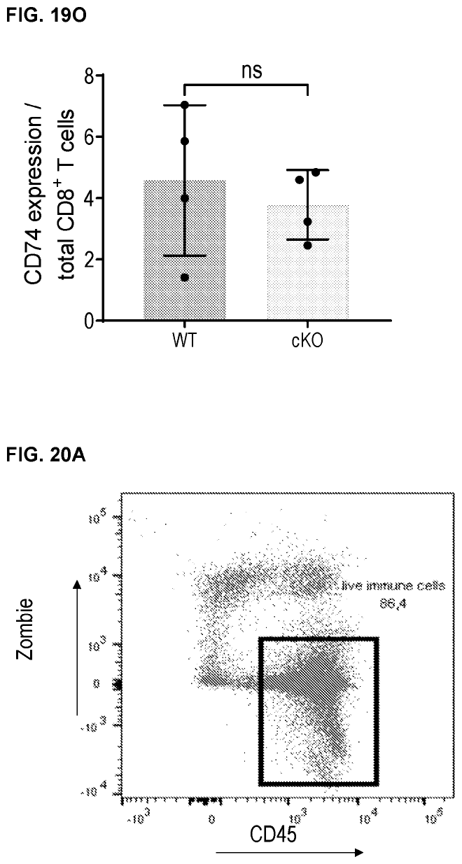

FIGS. 19A-19O illustrate that CD74 deficiency in DC specifically affects the dendritic cells population. Female 6-week-old CD11c-Cre×CD74flox×CD74 flox mice were sacrificed, spleens were harvested, processed to a single cell suspension, and total PBMCs were isolated. FIG. 19A graph shows the mean and SD percentage of monocytes out of total live cells (WT n=5, cKO n=5). FIG. 19B graph shows the mean and SD percentage of macrophages out of total live cells (WT n=5, cKO n=5). FIG. 19C graph shows the mean and SD percentage of B cells out of total live cells (WT n=5, cKO n=5). FIG. 19D graph shows the mean and SD percentage of dendritic cells out of total live cells (WT n=5; CKO n=5). FIGS. 19E-19H graphs show the mean and SD percentage of CD74 expression on monocytes (FIG. 19E), macrophages (FIG. 19F), B cells (FIG. 19G) and DCs (FIG. 19H) (WT n=6; cKO n=6). FIG. 19I graph shows the mean and SD percentage of CD4+ T cells out of total live cells (WT n=6; cKO n=6). FIG. 19J graph shows the mean and SD percentage of CD8+ T cells out of total live cells (WT n=6; cKO n=6).) FIG. 19K graph shows the mean and SD percentage of CD62L+ T cells out of total CD4+ T cells (WT n=6; cKO n=6). FIG. 19L graph shows the mean and SD percentage of CD103+ T cells out of total CD8+ T cells (WT n=6; cKO n=6). FIG. 19M graph shows the mean and SD percentage of FOXP3+ T cells out of total CD4+ T cells (WT n=4; cKO n=4). FIGS. 19N-19O show expression of CD74 in the CD4+ (FIG. 19N) and CD8+ (FIG. 19O) populations. ns p>0.05, ** P<0.005, *** p<0.0005.

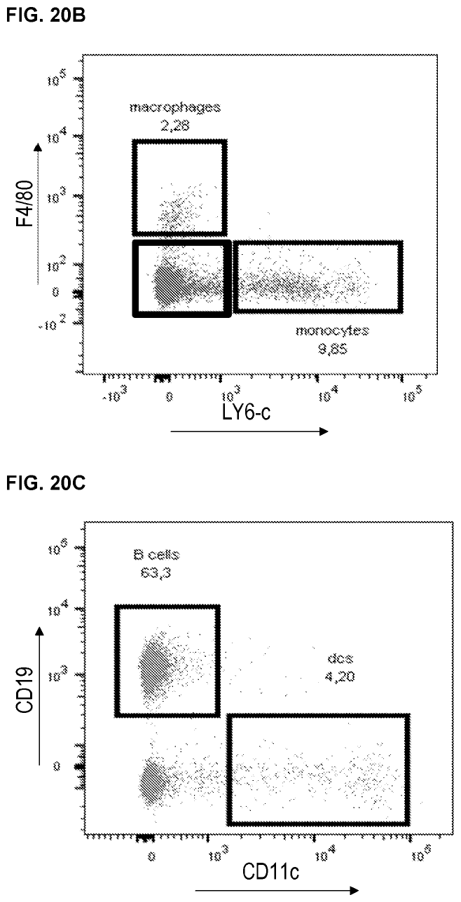

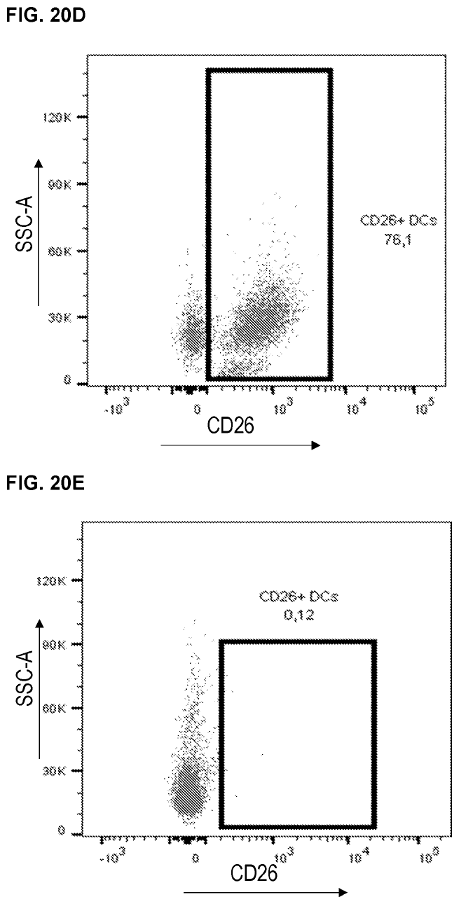

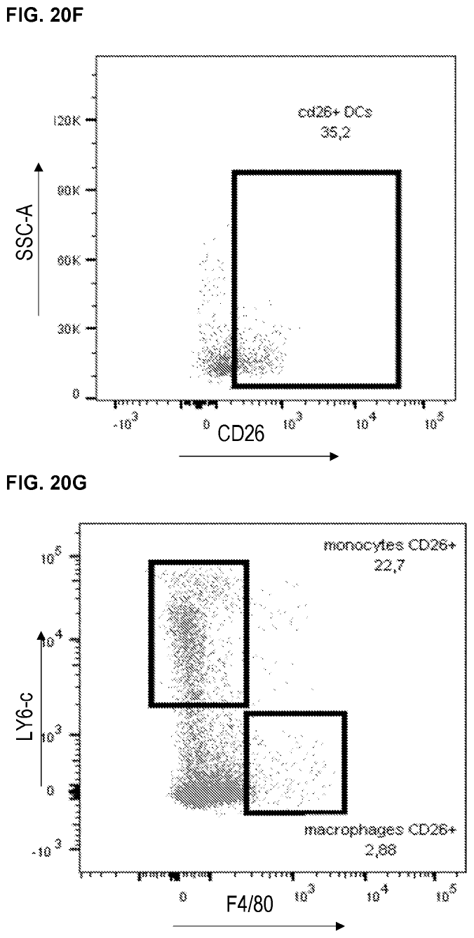

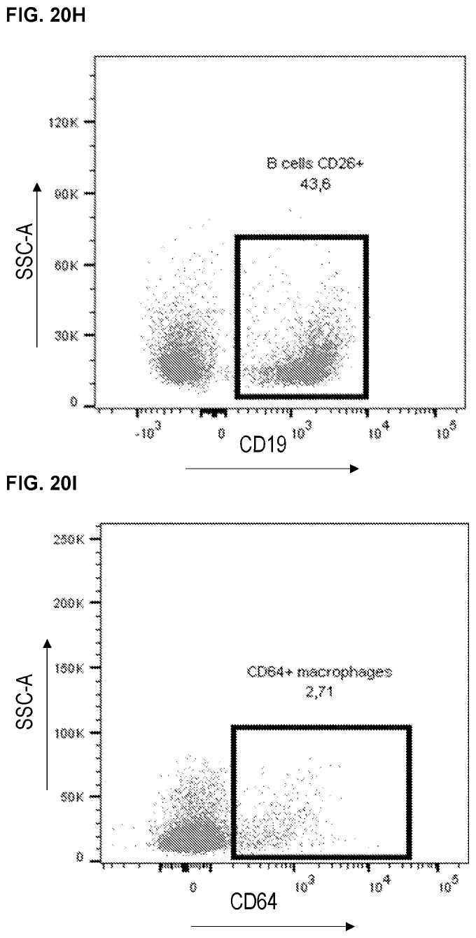

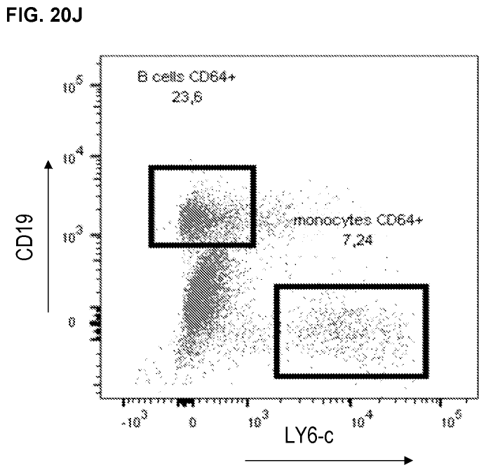

FIGS. 20A-20J illustrate that CD26 and CD68 are not specific markers for DCs or macrophages. PBMCs from the spleen of naïve mice were analyzed by flow cytometry. FIG. 20A shows that Dead cells were excluded from analysis by Zombie Live/Dead staining. FIG. 20B shows that macrophages and monocytes were gated for F4/80 and LY-6c, respectively. FIG. 20C shows that the double negative population was analyzed for CD19 and CD11c to detect DC and B cells. In FIG. 20D, DCs obtained in FIG. 20C were analyzed for CD26. FIGS. 20E-20F shows that the CD45+ population was gated for CD26 as a dendritic cell marker. FIGS. 20G-20H shows that CD26+ DCs were analyzed for F4/80, LY-6c and CD19. FIG. 20I shows that CD45+ population was gated for CD64 as a macrophage marker. In FIG. 20J, CD64+ macrophages were analyzed for CD19 and LY-6c expression.

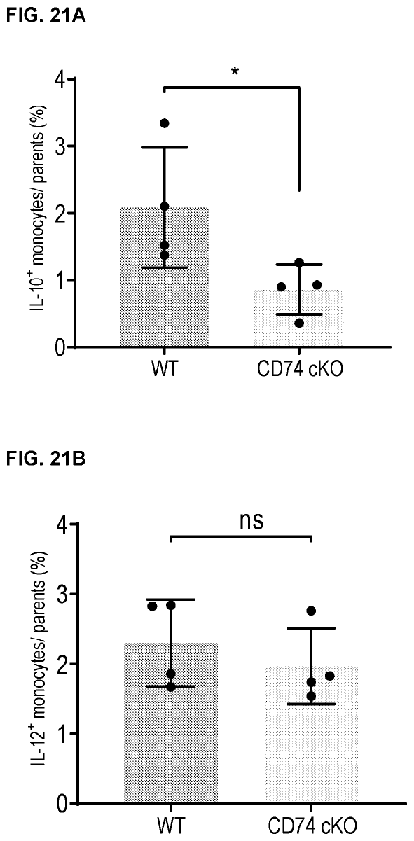

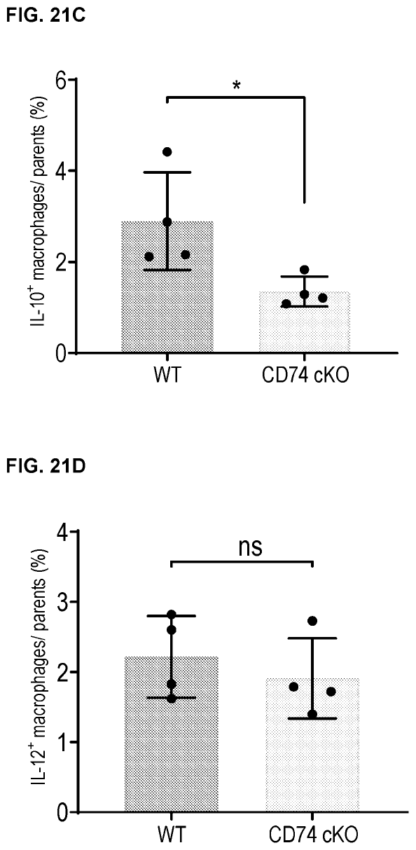

FIGS. 21A-21D illustrate that conditional KO of CD74 in mature B cells impacts the IL-10 release from monocytes and macrophages. Female 6-week-old CD23-Cre×CD74flox×CD74 flox mice were injected with 5*105 E0771 cells into each of the 4th mammary pads. After 21 days, mice were euthanized, and tumors were harvested, processed to a single cell suspension, and total PBMCs from the tumor site were isolated. Cells were then activated with PIM and analyzed by flow cytometry. FIG. 21A graph shows the mean and SD percentage of IL-10+ monocytes out of total monocytes (WT n=4, cKO n=4). FIG. 21B graph shows the mean and SD percentage of IL-12+ monocytes out of the total population (WT n=4, cKO n=4). FIG. 21C graph shows the mean and SD percentage of IL-10+ macrophages out of total macrophages (WT n=4, cKO n=4). FIG. 21D graph shows the mean and SD percentage of IL-12+ macrophages out of total (WT n=4, cKO n=4). (ns) p>0.05, (*) P<0.05.

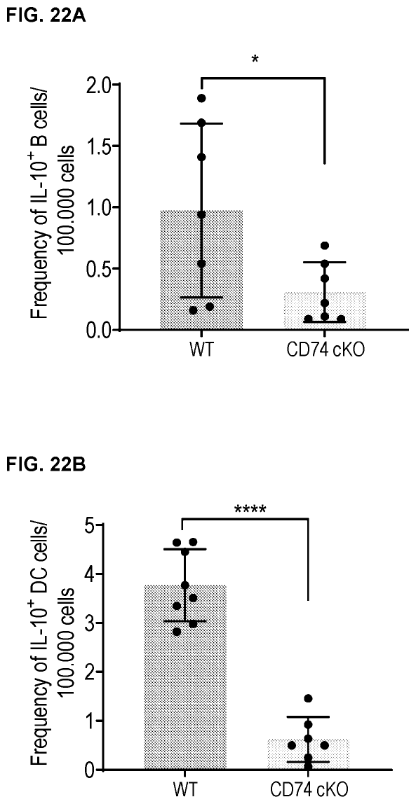

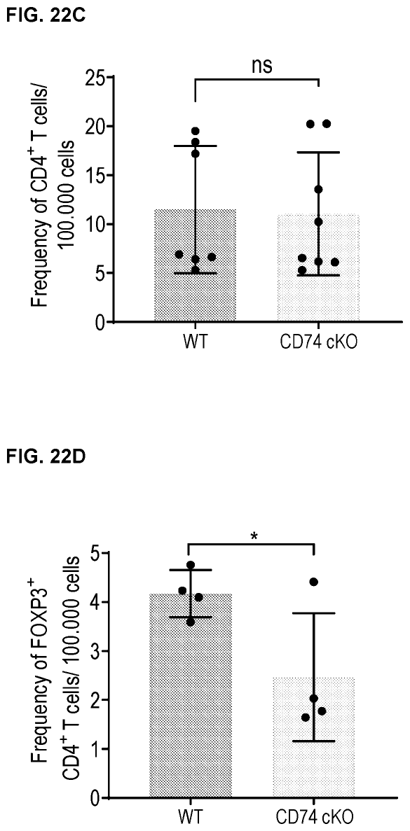

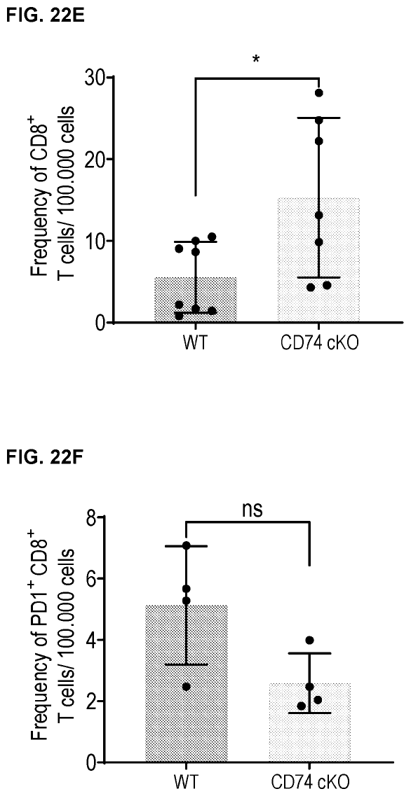

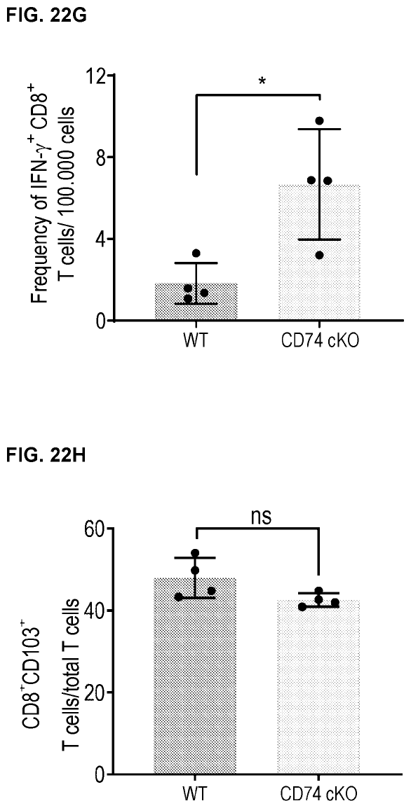

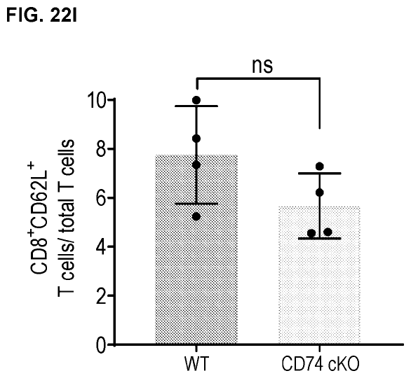

FIGS. 22A-22I illustrate that CD74 conditional KO in mature B cells reduces the frequency of tumor-infiltrating immunosuppressive cells. Female 6-week-old CD23-Cre×CD74flox×CD74 flox mice were injected with 5*105 E0771 cells into each of the 4th mammary pads. After 21 days, mice were euthanized, and tumors were harvested, processed to a single cell suspension, and total PBMCs from the tumor site were isolated. Cells were then activated with PIM and analyzed by flow cytometry. FIG. 22A shows frequency of IL-10+ B cells out of total B cells (WT n=7; cKO n=7). FIG. 22B shows frequency of IL-10+ DCs in the tumor site (WT n=8; CD74 cKO n=7). FIG. 22C shows frequency of CD 4+ T-cells (WT n=7; CD74 cKO n=8). FIG. 22D shows frequency of FOXP3+ T cells out of total CD4+ T cells (WT n=4; CD74 cKO n=4). FIG. 22E shows frequency of CD 8+ T-cells (WT n=8; CD74 cKO n=7). FIG. 22F shows frequency of PD1+ T cells out of total CD8+ T cells (WT n=4; CD74 cKO n=4). FIG. 22G shows frequency of IFN-γ+ T cells out of total CD8+ T cells (WT n=4; CD74 cKO n=4). FIG. 22H shows frequency of CD103+ T cells out of total CD8+ T cells (WT n=4; CD74 cKO n=4). FIG. 22I shows frequency of CD62L+ T cells out of total CD8+ T cells (WT n=4; CD74 cKO n=4). ns p>0.05, * p<0.05, **** p<0.00005.

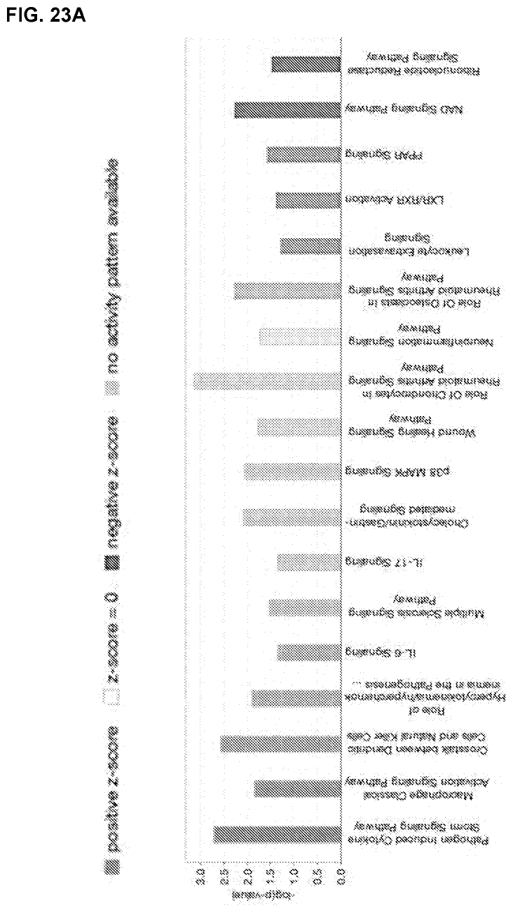

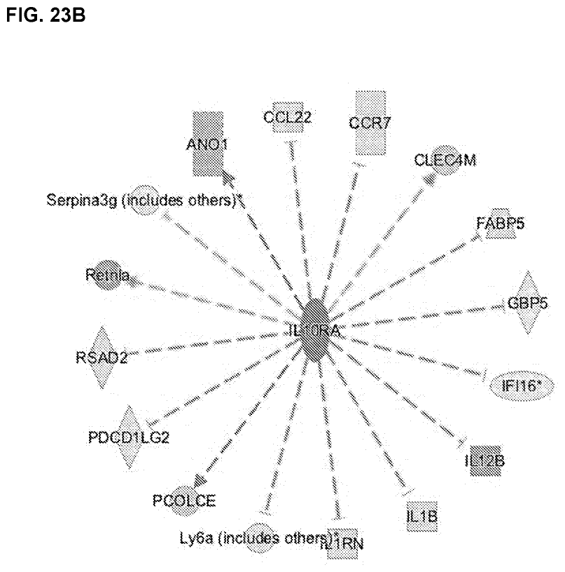

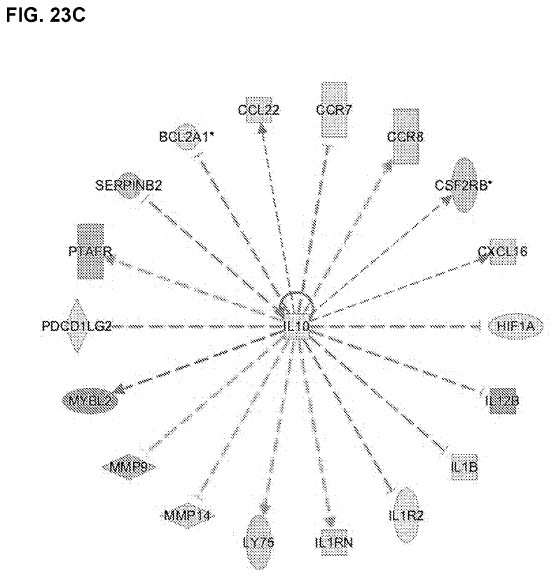

FIGS. 23A-23C illustrate that CD74 downregulation in DC induces pro-inflammatory pathways and depicts RNA-seq analysis. Female 6 weeks old C57BL/6 mice were injected with 5*105 E0771 cells into each of the 4th mammary pads (total of two mammary pads per mouse). After 10 days, DRQ was injected intravenously for 4 consecutive days (days 10-13). After 21 days, tumor sizes were measured, and mice euthanized. Tumors were processed into single cell suspension and DCs were sorted from the tumor microenvironment of mice treated either with PBS or DRQ. Four replicates were used from each group. FIG. 23A depicts visualization of the Ingenuity Pathway Analysis (IPA) where the relevant pathways are shown ordered by significance (p-value), calculated in IPA by right-tailed Fischer's exact t-test. The pro-inflammatory pathways show a positive z-score indicating that pathway activity is increased in DRQ versus PBS treated-mice. FIG. 23B shows IPA Upstream Regulator Analysis was used to predict the upstream regulators responsible for the gene expression changes observed. IL-10 receptor is shown to be downregulated in DCs treated with DRQ. FIG. 23C depicts the gene interactions, where some genes are upregulated, while some genes are suppressed. Genes related to immunogenic response of DCs are increased in DRQ versus PBS treated mice.

### Example 2

### Effect of DRQ in Melanoma

Higher MIF expression is correlated with worse outcomes in advanced melanoma patients. Analysis of the publicly available The Cancer Genome Atlas (TCGA) datasets revealed that high MIF expression was correlated with reduced overall survival in the skin cancer melanoma (SKCM) patient cohort (FIG. 1). Of the cohort of TCGA melanoma patients following any treatment, the level of MIF expression was significantly higher in those with poorer outcomes compared to those with response. Additionally, a query of SKCM patients who received anti-PD-1 demonstrated that high MIF expression was associated with decreased survival (de Azevedo et al. Oncoimmunology 9 (1): 1846915, 2020).

MIF is expressed by tumor cells and downregulated by DRQ. B16F10 cells were incubated for 48 hours with either vehicle control (Tris-HCl pH 8.5+8.5% sucrose) or DRQ at 25 μg and 50 μg and supernatants were collected. As determined by ELISA, MIF expression increased in the control group, whereas it decreased in a dose-dependent manner in the DRQ treated groups, as predicted (p<0.01, FIG. 2).

DRQ controls tumor growth in a localized intradermal tumor model. Eight-week-old C57BL/6 mice were injected with 5 x105 B16F10 mouse melanoma cells intradermally on day 1. Mice were then treated three times per week for two weeks with DRQ (at a dose of 100 μg) or vehicle control starting when the tumor was first visible (day 5). Per IACUC requirement, tumors were harvested at 2 cm diameter. DRQ provided a statistically significant survival benefit compared to vehicle control in these localized tumors (FIG. 3).

DRQ increases immune infiltration into the tumor microenvironment. Photomicrographs of an intradermal B16F10 melanoma tumor from a mouse treated with DRQ demonstrated a brisk immune infiltrate within the tumor (FIG. 4) compared with vehicle control. The tumors were disaggregated and then analyzed with flow cytometry. Flow data demonstrated a notable infiltrating immune population, and ~11% of the immune population consisted of CD8+ T lymphocytes; while similar tumor samples with vehicle control had significantly fewer identifiable immune cells by flow cytometry.

DRQ increases the fraction of TRP2-reactive CD8+ cells in B16F10 tumors. B16F10 tumors from DRQ versus vehicle control-treated mice were incubated with TRP2-PE tetramers and analyzed with flow cytometry (FIG. 5). DRQ caused a significant increase in TRP2-reactive CD8+ infiltrating lymphocytes within the tumor (30.8%) compared to vehicle control (2.7%).

RNA-Seq data demonstrates DRQ decreases expression of ERK in B16F10 cells. Bulk RNA-sequencing was performed on DRQ-treated B16F10 cells versus control to confirm that DRQ functions through reduction of ERK expression and to identify other genes differentially expressed. Read counts were analyzed to assess differences in gene expression between populations using DEseq2. Count data was fitted to a negative binomial general linear model; to control for multiple comparisons a Benjamini-Hochberg correction was performed. Notably, the DRQ samples had significantly reduced levels of MAPK1 (ERK2) expression compared to control (p<0.05). DRQ also downregulates TLR-2 and TLR-4 and other proinflammatory messengers, that play a key role in the inflammatory reaction.

DRQ downregulates pERK and pSTAT3 expression. B16F10 cells were grown in culture and then incubated with either vehicle or 50 μg of DRQ for 1 hour. Half a million cells from each condition were spun and lysed. Lysates were collected and subjected to SDS-PAGE in 10-20% gradient gels under reducing conditions. After electrophoresis, proteins were transferred to PVDF to evaluate phosphorylated ERK1/2 (pERK 1/2) and pSTAT3. DRQ downregulated phosphorylated pERK1/2 and pSTAT3 (FIG. 6).

Toxicology Evaluation: A formal toxicity study was conducted. The maximum feasible dose (MFD) of 25 mg/kg was well tolerated even when given daily for 7 days i.v. to male and female C57BL/6 mice. There were no clinical signs and no effects on body weight and no lesions in gross necropsy.

### Example 3

### Determining Minimum Effective and Optimal Doses of DRQ

Studies to determine minimum effective and optimal dose of DRQ (DRhQ or DRmQ) in BRAF wt mouse melanoma models B16F10 and YUMM4.1 are performed. The minimum effective dose is the dose at which this the dose that shows least tumor growth with acceptable toxicity. Animals are thoroughly assessed for signs of toxicity in this model.

The mice are observed for status, weight, and food intake at least daily. On death of the animal or endpoint of the study, a full necropsy with organ weights, histopathology, hematology, and clinical chemistry is conducted. For optimal dose and dose range finding studies, two mouse melanoma models are utilized (B16F10 and YUMM4.1). Male and female C57BL/6 mice at 8-9 weeks of age are injected intradermally with 1×10⁵ mouse melanoma cells on the flank. Upon engraftment and detection of visible tumor, the mice begin treatment with either DRQ i.v. (at doses of 1 μg, 10 μg, 25 μg, 50 μg, 100 μg, 250 μg, and 500 μg (500 μg is the MFD) or vehicle control, three times per week for two weeks. Tumor growth is monitored by conventional caliper daily and mice are euthanized when the tumor diameters reach 2 cm. Tumors are harvested to generate FFPE slides for cyclic multiplexed immunofluorescence (cycIF) and for flow cytometry and protein and transcriptional interrogation. Cells are sorted into CD45+ fraction (immune cells) and tumor fraction (CD45-cells). Each fraction is then analyzed for expression of CD74, CD44, PD-L1, PD-L2, HIF1a, MIF and pERK1/2 by Western blot. Soluble CD74 (sCD74) in plasma at time of sacrifice is also measured.

### Example 4

### Assessment of Mechanisms Associated with Durable Tumor Control by DRQ

Male and female 8-9 week-old C57BL/6 mice are injected intradermally with 1×10⁵ mouse melanoma cells on the flank and on engraftment and detection of visible tumor, mice are treated three times per week for two weeks, with: 1) vehicle control or 2) DRQ (DRhQ or DRmQ): (2 experimental groups of each mouse background×2 cell lines×10 mice/group×2 sexes=80 mice). Tumors are harvested at 2 cm diameter; a portion used for FFPE slides for cycIF and the remainder dissociated. Cells are sorted into CD45+ fraction and tumor fraction as in Example 2. Specific-anti tumor immunity is measured by tetramer staining among CD8+ T cells using flow cytometry. TRP1, TRP2, and gp100 are melanoma-specific antigens that have tetramers available (Immudex). Additionally, number of infiltrating CD8+ T cells, CD4+ T cells, MDSCs, NK cells and Tregs is assessed using flow cytometry. Cell surface expression of CD74, CD44, PD-L1, and PD-L2 is assessed by flow cytometry. pERK1/2, MIF, and total CD74 is analyzed by Western blot. Single cell RNA-seq (scRNA-seq) is performed on representative mice from each group. The cycIF and RNA-seq experiments allow identification of the impact of DRQ on all immune cells.

Multiplexed Immunofluorescence: Cyclic multiplexed Immunofluorescence (CycIF) exploits in situ hybridization of complementary oligonucleotides for labeling and to facilitate signal removal for sequential rounds of tagging and imaging. CycIF is therefore able to visualize endogenous protein expression while maintaining spatial context in situ. This can allow for imaging of >20 unique epitopes simultaneously (see Table 1 for targets).

TABLE 1
List of antibodies for CycIF and Ab-Seq

| | | | |
|---|---|---|---|
| CD117 | CD11b | CD11c | CD138 |
| CD14 | CD15 | CD16 | CD161 |
| CD19 | CD193 | CD1d | CD11a |
| CD206 | CD25 | CD3 | CD4 |
| CD45RA | CD45RO | CD56 | CD62L |
| CD68 | CD69 | CD8 | CD80 |
| CD86 | PD-L2 | CD74 | PD-L1 |
| LAG3 | PD1 (CD279) | CTLA-4 | TIM-3 |

Image processing, data and statistical analysis: The multiplexed images are processed and analyzed using FCS Express 6 Image Cytometry software packages. Wilcoxon signed rank tests is utilized to determine statistically significant differences in paired data sets, followed by the Spearman correlation coefficient to assess for correlations of cell percentages and densities among cell lineages. For spatial analysis, the point pairwise correlation function, Ripley's K, is utilized to determine the spatial correlation among subtypes of immune cells. P values are adjusted for multiple comparisons using the false discovery rate (FDR) of Benjamini and Hochberg.

Overview of single-cell approach: scRNA-seq profiling assigns high-resolution molecular identities by generating high-confidence gene expression levels. Each single cell is also assessed for 32 proteins by epitope (Table 1), matching them to canonical immune classes. BD Rhapsody single cell platform is used, followed by standard Illumina sequencing. The BD Rhapsody pipeline is used for initial quality control and filtering, batch correction, read alignment, and to generate gene and protein epitope count matrices. Seurat R bioinformatics package is used for more detailed analyses including clustering/identification of subpopulations.

Single cell preparation: Tumor tissues are dissociated with collagenase IV and hyaluronidase for one hour. The resulting cells are stained with anti-CD45 antibody and a cocktail of AbSeq antibodies (BD AbSeq; BD Bioscience, Table 1). Cells are stained with 7-AAD viability marker and sorted for CD45+ and tumor fractions. ~10,000 total cells are then be loaded from each flow sorted sample onto a BD Rhapsody Cartridge for single cell capture: 5000 CD45+ immune cells and 5000 tumor cells.

cDNA library preparation and sequencing: The BD Rhapsody System is used for single-cell capture and cDNA preparation and amplification. Final pooled libraries are sequenced (100 bp paired-end) on a NovaSeq 6000 sequencer to a sequencing depth of 100,000 reads per cell for the WTA mRNA library and 32,000 reads per cell for the AbSeq library (1000 reads per cell per antibody with 32 antibodies).

Data Analysis and QC: The raw single cell RNA-seq FASTQ files are processed following the BD Biosciences Rhapsody pipeline where reads are aligned to a reference genome using Bowtie2, and gene and protein epitope count matrices are generated. The distribution-based error correction (DBEC)-adjusted molecule counts are used for all analyses using the R package Seurat 3.0. Expression matrices are log-normalized. Uniform Manifold Approximation and Projection (UMAP) are used for dimensionality reduction. A negative binomial generalized linear model is implemented using the Seurat R package sc transform.

Detection of transcriptional markers: Once immune populations are determined, differential gene expression (DE) is performed for subpopulations applied to average gene expression value, appropriately weighted for number of cells, using R package Seurat. Genes that are co-regulated are identified by building gene co-expression networks based on the Mutual Information (MI) criteria.

### Example 5

### Materials and Methods for Assessment of CD74 as a Therapeutic Target in Triple-Negative Breast Cancer

Mice: C57BL/6, CD74^{-/-}, Vert-x, CD23-cre×CD74-flox, CD11c×CD74-flox mice were used in this study. Vert-x mice were provided by C. Mauri, UCL. All animals were used at 6-8 weeks of age. In the breast cancer model, only females were used, and the groups were age and sex-matched in each experiment. All animal procedures were approved by the Animal Research Committee at the Weizmann Institute of Science. To generate Cre-CD23×flox-CD74 littermates, Cre-CD23 and flox-CD74 mice were crossed, and screened by PCR for CD74 and CD23 genotypes. To generate Cre-CD11c×flox-CD74 littermates, Cre-CD11c and flox-CD74 mice were crossed, and screened for CD74 and CD11c genotypes by PCR.

Breast cancer induction: E0771 cell-line cells were grown in a complete RPMI medium with 10% fetal bovine serum. For tumor models, 5*10⁵ cells in PBS were injected s.c. to each of the fourth mammary pads (total 2 mammary pads/mouse) of 6-8-weeks old C57BL/Vert-x competent female mice.

Tumor load measurements: Tumor size was assessed by external measurement of the length (L) and width (W) of the tumors in two dimensions using a Vernier caliper. Tumor volume (V, expressed in mm³) was calculated by using the following equation: V=(L×W²/2, when W is the shorter dimension measurement, and L is the longer).

Preparation of Tumor infiltrating Lymphocytes (TIL): Tumor tissues were harvested 21 days following tumor implantation, cut into small pieces, and incubated in digestion buffer (1 mg/ml collagenase A, 0.15 mg/ml Hyaluronidase, 10% FBS, 1% P/S) for 45 minutes in a 37 °C incubator with gentle shaking. Tumor tissue was then passed through a 100 μm cell strainer and washed 3 times with PBS. Dissociated tumors were then suspended in 8 ml 44% Percoll solution and loaded onto 5 ml 67% Percoll cushions. Samples were centrifuged for 20 minutes at 1000 RCF with no brake at room temperature. The middle fraction containing infiltrating mononuclear cells was collected and washed twice with PBS.

B cell isolation from spleen and bone-marrow: Murine spleens were dissected post-mortem and collected in PBS. Organs were processed through a 100-μm-cell strainer, and treated with Red Blood Lysis buffer to lyse erythrocytes for 5 minutes. Next, cells were washed with PBS, and processed through a 40-μm-cell strainer. Finally, B cells were purified by positive B cell selection with B220 magnetic beads.

Immune cell isolation from spleen: Murine spleens were dissected post-mortem and collected in PBS. Organs were processed through a 100-μm-cell strainer, and treated with Red Blood Lysis buffer for 3 minutes. Cells were then washed with PBS and processed through a 40-μm-cell strainer.

Regulatory B cell activation: For detection of IL-10 on B cells, B cells at 2.5×10⁶ cells/ml in complete ISCOVE medium were cultured for 5 hours with PMA (100 ng/ml), Ionomycin (1 μg/ml), Monensin (1 μg/ml), and LPS (10 μg/ml).

Tolerogenic DC cell activation: For detection of IL-10 on DC cells, DC cells at 2.5×10⁶ cells/ml in complete ISCOVE's medium were cultured for 5 hours with PMA (100 ng/ml), Ionomycin (1 μg/ml), Monensin (1 μg/ml), and LPS (10 μg/ml).

Co-cultures: E0771 cancer cells were seeded in 12-well plates. The next day, B cells were purified from splenocytes by positive B cell selection with B220 magnetic beads. B cells were then cultured either alone, or co-cultured in 12 well plates in complete ISCOVE's medium with 10% FBS for 24 hours at ratios of 1:5 of B cells/E0771 cells. Similarly, DC were purified from splenocytes by positive Mojosort mouse Pan Dendritic cell isolation kit and added to the E0771, or cultured alone in 12 well plates with 10% FBS complete RPMI medium for 24 hours at a ratio of 1:3. The total number of cells in each well was 2.5*10⁶ under all conditions. For the last 5 hours of culture, cells were activated PMA, Ionomycin, Monensin and LPS.

Flow cytometry staining: FACS analysis was performed using FACS Canto. FACS data analysis was performed using FlowJo software. Antibodies are listed in Table 2, below. Cells were stained using specific antibodies for surface markers as previously described, followed by fixation, and permeabilization using BD Cytofix/Cytoperm commercial kit or eBioscience transcription factor staining buffer set. Cells were then stained with intracellular antibodies.

CD74 blocking with DRQ-2 in-vivo: Blocking of CD74 in vivo was performed using DRQ and 20 mM TRIS buffer, pH8.5 in saline as a control. Treatment with DRQ or PBS was started at day 10 after tumor cell administration and continued for 5 consecutive days. The inhibitor or control were injected into the tail vein (100 μg/100 μl per mouse).

CD74 blocking with LN-2 antibody in-vitro: B cells were treated with LN-2 blocking antibody or IgG isotype control (150 μg/ml) for 24 hours. The total number of cells in each well was 5*10⁶ under all conditions.

MIF activation: Cultures of 5×10⁶ cells were activated with 150 ng/ml of MIF activator in 1 ml medium in a 24 well plate for 24 h.

In vitro DC suppression assay: DCs were isolated from the spleen of WT and CD74^{-/-} mice through the positive Mojosort mouse pan dendritic cell isolation kit. Isolated DCs were cocultured for 24 h with E0771 cancer cells at a 1:3 ratio. Splenic CD3⁺ T cells were isolated using the CD3⁺ mouse positive selection kit. T cells were labeled with Carboxy Fluorescein Succinimidyl Ester (CFSE) and seeded at ratios of 1:1 with DCs, in the presence of anti-CD3 coupled beads for 72h. Cells were then collected, and T cells analyzed for proliferation by FACS.

SP1 blocking in-vitro: E0771 cancer cells were seeded in 12-well plates. The next day, DCs were purified from splenocytes and then co-cultured with the tumor cells, at an E0771/DC cell ratio of 1:3. Cells were cultured in complete RPMI medium+10% FBS, in the presence of 20 μM of Mithramycin, or DMSO as a negative control for 48 hours. The total number of cells in each well was 2.5*10⁶ under all conditions.

IL-1β activation in-vitro: E0771 cancer cells were seeded in 12-well plates. The next day, DCs were purified from splenocytes and then co-cultured with the cancer cells, at an E0771/DC cell ratio of 1:3. Cells were cultured in complete RPMI medium+10% FBS, in the presence of 20 nM of IL-1β recombinant antibody or PBS as negative control for 48 hours. Total number of cells in each well was 2.5*10⁶ under all conditions.

RNA extraction for high throughput experiments and RNA-sequencing: Tumor infiltrating DCs were sorted from dissociated TME. mRNA was extracted from these cells using the Dynabead mRNA purification kit, and Illumina libraries were constructed from total mRNA using the bulk adaptation of the MARS-Seq protocol for Illumina TruSeq RNA Sample Preparation v2 (Cat. no. RS-122-2002, Illumina) according to the manufacturer's instructions. Indexed samples were sequenced in an Illumina NextSeq High output HiSEq 2500 machine in single-read mode. STAR (2.7.3a) TopHat (v2.0.10) was used to align the reads to the Mus_musculus genome (GRCm39) and human genome (hg19). Counting reads based on annotations downloaded from Ensembl (release 106) on hg19 RefSeq genes was done with HTSeq-count (version 0.11.2) (v0.6.1p1). Differentially expressed genes were identified using DESeq2 with the betaPrior, cooksCutoff, and independent filtering parameters set to False. Raw P values were adjusted for multiple testing using the procedure of Benjamini and Hochberg. Differentially expressed genes were determined by a p-adj of <0.05 and absolute fold changes >1.5 and max raw counts >10.

RNA extraction and cDNA synthesis for RT-qPCR: Total RNA was isolated from cells using the TRI Reagent® RNA Isolation Reagent, according to the manufacturer's instructions. For cDNA synthesis, 500 ng or 1 μg mRNA was used with the qScript™ cDNA Synthesis Kit, according to the manufacturer's instructions.

qRT-PCR: qRT-PCR was performed on the Lightcycler 480. The program used was: 10′ at 95 °C, followed by 45 cycles of amplification (95 °C for 10″, 60 °C for 10″, 72 °C for 10″) and then cooling to 4 °C. Primers are listed in Table 3.

siRNA transfection: siRNA was introduced by electroporation using a Nepagene (Ichikawa, Chiba, Japan) Super Electroporator NEPA21 Type II, using 2 mm gap cuvettes, with 20 μg of siRNA at 225 mv, 5 msec in 100 μl of OptiMem medium. After the transfection, the cells were resuspended in RPMI 1% FCS medium, and incubated for 24 hours.

ChIP qPCR: ChIP-seq was performed as previously described [20]. For each sample, 5×10⁵ tumor-infiltrating DC cells were sorted and activated with rmMIF or with vehicle for 1 h, then cross-linked with DSG (disuccinimidyl glutarate) and fixed. Chromatin was immunoprecipitated with anti CD74 or anti-SP1 antibodies and ChIP-DNA was processed. The samples were analyzed by qPCR for SP1 or IL-1β promotor.

Statistical analysis: Data analysis was performed using Graphpad Prism (Version 7.0 f, GraphPad Software, Inc., La Jolla, CA, USA). For most experiments, the mean is provided together with SEM or SD. To determine the significance of the differences, 2-way ANOVA and Student's t test, either one- or two-tailed and 2-way, were used depending on the experiment. Results were deemed significant with a P value of 0.05 or less.

TABLE 2
List of antibodies for flow cytometry

| Antibody | Fluorophore | Company |
|---|---|---|
| CD19 | PE-cy7 | Invitrogen |
| | BV711 | Biolegend |
| B220 | FITC | Biolegend |
| | PB | Biolegend |
| F4/80 | PB | Miltenyi |
| | FITC | Biolegend |
| | BV711 | Biolegend |
| Ly6-c | BV711 | Biolegend |
| IL-10 | PE | Biolegend |
| | BV-421 | BD |
| CD80 | Pe-cy7 | Biolegend |
| | FITC | Biolegend |
| CD11C | PE | Biolegend |
| IL-12 | Pe-cy7 | Biolegend |
| CD4 | FITC | Biolegend |
| CD8 | PE-cy7 | Invitrogen |
| FOXP3 | PE | Thermofisher |
| IFN-γ | PB | Biolegend |
| CD74 | FITC | R&D System |
| ZOMBIE | APC-Cy7 | Biolegend |
| | PB | Biolegend |
| CD3 | FITC | Biolegend |
| IL-1β | APC | Termofisher |
| CD45 | PERCP | Thermofisher |

TABLE 3
List of primers for qRT-PCR

| Organism | Gene | Forward | Reverse |
|---|---|---|---|
| Mouse | SP1 | TTG AAA AAG GAG TTG GTG | TGC TGG TTC TGT AAG TTG |
| | | GC | GG |
| Mouse | IL-1β | GAAATGCCACCTTTTGACAGTG | TGGATGCTCTCATCAGGACAG |
| Mouse | IL-10 | ATTTGAATTCCCTGGGTGAGAAG | CACAGGGGAGAAATCGATGACA |
| Mouse | MIF | GTTTCTGTCGGAGCTCAC | AGCGAAGGTGGAACCGTTCCA |
| Mouse | SP1 Promoter | CTTGCCTCGTCAGCGTCC | GTGGACTCATCCTTACCGCTC |
| | Area | | |
| Mouse | IL-1β Promoter | AAGAGGCTATTGCTACCCTGA | TGTCCAACTTGTTTTCCCTCCC |
| | Area | | |

### Example 6

### CD74 Regulates Tumor Load by the Control of Immune-Suppressive Populations in the TNBC Murine Model

To determine the in vivo role of CD74 in the ME of TNBC, E0771 murine TNBC cells were orthotopically injected into C57BL/6 or CD74 deficient (CD74^{-/-}) mice. Tumor size was monitored every 5 days from the day of injection, and mice were sacrificed on day 21. As shown in FIGS. 7A-7C, the absence of CD74 significantly reduced tumor development and growth. Next, the expression of CD74 on cells in the TME were analyzed. CD74 was widely expressed on immune cells, but its expression was upregulated on the tolerogenic populations of DCs and B cells (FIG. 15A). Furthermore, CD74 expression on tumor-infiltrating B cells and DCs was significantly higher compared to its levels on the peripheral splenic populations (FIGS. 15B-15C), suggesting a role for CD74 in the TNBC microenvironment.

Next, the role of CD74 in cells derived from the tumor microenvironment were determined. TNBC cells reprogram their microenvironment towards an immunosuppressive phenotype by inducing the secretion of IL-10 in the various immune cell populations [5]. Therefore, the antigen-presenting cells (APCs) and T cells in the TME derived from WT and CD74-deficient mice were analyzed.

DCs positively or negatively regulate the anti-tumor immune response according to the cytokines released and the expression of costimulatory molecules able to bind their T cell counterparts in order to induce their priming [36]. Thus, DCs in the tumor microenvironment in WT and CD74-deficient mice were analyzed for their numbers and functionality by FACS analysis. As shown in FIG. 7D, a significantly higher percentage of CD74 deficient DCs were observed in the TME. These accumulated cells expressed higher levels of CD80 (FIG. 7E) and a lower percentage of IL-10 (FIG. 7F; FIG. 17B), suggesting that the CD74 deficient DC in the TME were more immunogenic and less tolerogenic (gating strategy appears in FIGS. 16A-16I). Furthermore, the percentage and frequency of B regs (gating strategy appears in FIGS. 16J-16M; frequency appears in FIG. 17A), of IL-10⁺ macrophages, regulatory T cells, CD4⁺ T cells and CD8⁺ exhausted T cells were downregulated. No differences were detected in the frequency of CD8⁺ T cells or CD8⁺ CD62⁺ T cells, but the cytotoxic activity of CD8⁺ T cells was induced in the TME of CD74 KO mice (FIGS. 17C-17I).

Since deficiency of CD74 results in a reduced number of mature B cells and CD4⁺ T cells [37], next it was determined whether the reduced tumor load detected in the CD74 deficient mice results from its function as a MIF receptor, or whether it is due to the role of CD74 in antigen presentation, controlling T cell numbers. To address this question, CD74 function was blocked in C57BL/6 mice, and the infiltration of immune cells and their activity were analyzed. To this end, TNBC cells were injected to the mice. Starting from day 10, mice were intravenously treated for 5 consecutive days (10-14), with either a partial MHC class II construct, which inhibits ligand binding to CD74 (DRQ) or vehicle (saline) control. Blocking CD74 reduced tumor growth and tumor volume (FIGS. 7J-7K). Analysis of immune cells in the TME showed that this treatment elevated the percentage of immunogenic DC, resulting in a downregulation of tolerogenic IL-10⁺ DC cells (tol-DCs; FIG. 7L; FIG. 18B) and upregulation of the IL-12 expressing DC (FIG. 7M) in the TME. Furthermore, a decrease in infiltrating Bregs and Tregs was observed in the DRQ-treated mice (FIGS. 7N-7O; FIGS. 18A, 18C, 18D) as well as an upregulation in the frequency of CD8⁺ T cells (FIG. 18E). These results suggest that MIF binding to CD74 positively regulates the tumor-suppressive ME in TNBC.

### Example 7

### MIF Induces the CD74 Immunosuppression of Cells of the TME

To determine whether CD74 can induce an in vitro expansion of tol-DC and Breg in presence of the cancer cells, a co-culture experiment was performed. Purified splenocytes derived from naïve WT and CD74 KO mice were cultured either alone or in the presence of E0771 cells. Cells were analyzed by FACS after 24 hrs. The presence of cancer cells induced the expansion of both tol-DC (FIG. 8A) and the Breg population (FIG. 8B). This expansion was abrogated in the presence of immune cells lacking CD74, emphasizing the importance of CD74 as an immunosuppressive pro-oncogenic factor.

Since MIF is the ligand of CD74, next it was determined whether MIF secreted from TNBC cells contributes to the expansion of immunosuppressive DCs and Bregs. Purified DC or B cells were separately cultured in presence of E0771 cells and activated with mouse recombinant MIF (rmMIF) or vehicle for 24 h. As shown in FIGS. 8C-8D, MIF induced a modest expansion of IL-10 positive tol-DCs (FIG. 8C) and Bregs (FIG. 8D). Since cancerous cells endogenously produce and release MIF, it was directly determined whether MIF derived from the malignant cells regulates immunosuppressive cell expansion. To this end, MIF expression in the E0771 cells was knocked down by MIF siRNA (FIG. 8E). Cancer cells expressing low or high levels of MIF were then cultured with DC or B cells, and their phenotype was analyzed. Downregulation of MIF expression resulted in a significantly reduced expansion of tol-DCs (FIG. 8F) and B-regs (FIG. 8G). Thus, the MIF secreted from the malignant cells plays a crucial role in the regulation of tol-DC and Breg expansion.

### Example 8

### The Effect of CD74 on Tumor Growth is Intrinsic to Dendritic Cells

To determine which antigen-presenting cells (B or DCs) lacking CD74 regulate tumor growth, CD74 was exclusively downregulated in mature B cells and DCs using conditionally CD74/Cre-flox mice (cKO). Specifically, WT mice lacking CD74 uniquely in CD23⁺ mature B cells and mice lacking CD74 in the CD11c⁺ dendritic cell population were injected with E0771 tumor cells, tumor size was monitored weekly, and mice were sacrificed on day 21. The lack of CD74 in mature B cells did not affect the tumor load (FIG. 9A) nor the phenotype of the infiltrating DC. Similar IL-10 (FIG. 9B) and IL-12 (FIG. 9C) levels were detected in DCs derived from both WT mice and animals deficient in CD74 in their B cell population. However, deficiency of CD74 in the mature B cell population resulted in a significant decrease of Bregs in the TME (FIG. 9D), suggesting a direct role for CD74 in the regulation of Bregs. To further investigate the role of CD74 knockdown in CD23⁺ mature B cells on DCs, in vitro co-culture of splenic DC cells derived from WT or CD23 cKO with or without E0771 cells was performed. Deficiency of CD74 in the mature B cell population had no effect on IL-10 release from naïve or cancer-activated DCs (FIG. 9E), while the co-culture of B cells with E0771 cells led to a weaker induction of Bregs (FIG. 9F).

To follow the role of CD74 in DCs, CD74 expression was downregulated in CD11C+ cells. The specificity of CD74 deletion to DCs was first validated by analyzing the accumulation of CD11c expressing population and their CD74 expression (FIGS. 19A-19H), and by evaluating the effect of the cKO on the T cell population (FIGS. 19I-19O) in the naïve mice. The downregulation of CD74 expression was specific to the DC population (FIG. 19H and gating strategy in FIGS. 20A-20K). In contrast to the limited effect of CD74 deficiency in B cells on the tumor load, deficiency of CD74 in DCs remarkably reduced tumor growth. Mice deficient in CD74 in the CD11c population developed significantly smaller tumors compared to their size in WT mice (FIGS. 10A-10C). DCs lacking CD74 in the TME displayed a decrease in their IL-10 levels (FIG. 10D, FIG. 22B), demonstrating a direct role for CD74 in the regulation of the tolerogenic DC phenotype. In addition, these mice showed a reduced accumulation of IL-10⁺ monocytes and macrophages and no difference in their IL-12 release, (FIGS. 21A-21D) Bregs (FIG. 10E, FIG. 22A) and T-regs (FIG. 10F, FIG. 22D) and an increase of CD8⁺ T cells in the TME (FIG. 10G, FIG. 22E) characterized by a more cytotoxic and a less exhausted phenotype (FIGS. 10H-10I, FIGS. 22F-22G). No differences were detected in the frequency of CD4⁺ T cells (FIG. 22C) and in the frequency of CD8+CD103+ or CD8+CD62L+ cells (FIGS. 22H-22I).

To further validate the effect of CD74 knockdown in DCs on B cells, in-vitro co-cultures were conducted. Splenic DCs or B cells purified from the CD11c cKO mice and cultured with the E0771 cell line, revealed a reduction of both tol-DCs (FIG. 10J) and Bregs (FIG. 10K), an effect that was not observed in the CD23 CKO mice. Thus, blocking of CD74 in CD11c⁺ cells reduced the levels of tol-DCs, Bregs and Tregs and their immunosuppressive functions in vivo and in vitro. Taken together, these results suggest that CD74 expressed in DCs, but not in B cells, is crucial for the regulation of immune cell suppression at the tumor site.

### Example 9

### CD74 Expressed in DCs Mediates the Crosstalk with the Bregs

To further assess the CD74-mediated crosstalk between tol-DCs and Bregs, WT, CD74 deficient DCs and B cells were cultured together in vitro in the presence of E0771 cells. CD74 deficiency in both cell types reduced the expansion of tol-DCs (FIG. 11A) and Bregs (FIG. 11B) in a synergistic manner. However, while the lack of CD74 in the B cell population did not affect the Bregs or the tolerogenic DC, DCs lacking CD74 reduced not only the expansion of the tol-DCs (FIG. 11A) but of the Bregs as well (FIG. 11B) suggesting a role of CD74 in governing the Breg expansion. To determine whether DCs directly regulate Breg expansion, WT and CD74^{-/-} DCs were cultured with E0771 cells for 24 h. Naïve B cells were then seeded with the previously activated DCs. As shown in FIG. 11C, the lack of CD74 in DCs strongly reduced IL-10 release in B cells, further supporting the role of CD74 on DCs in mediating Breg expansion.

To understand whether B cells can affect the DC phenotype, B cells were incubated with anti-CD74 blocking, LN-2, or IgG control antibodies, and activated with E0771 cells for 24 h. Naïve DCs were then seeded with the previously cancer-activated B cells. CD74 inhibition did not regulate the expansion of tol-DC (FIG. 11D).

To further confirm the immunosuppressive role of CD74 in DC activity, their function in T cell proliferation and suppression was analyzed. WT naive splenic CD3⁺ T cells were stained with the Cell Proliferation Dye (CPD), and then co-cultured with WT or CD74 KO splenic DCs, previously activated with E0771 cells. Downregulation of CPD expression in T cells correlates with the proportion of cells that undergo division. Induced CD8⁺ T cell proliferation was detected in the cells incubated with CD74 KO DCs (FIG. 11E). Moreover, lower levels of Tregs were observed in the presence of CD74KO DCs, further supporting an immunosuppressive role of CD74 expressed in DCs in the context of TNBC (FIG. 11F). In addition, the cytotoxicity of CD8⁺ T cells was assessed by analyzing interferon-γ (IFN-γ) protein levels. Elevated levels of IFN-γ were detected in T cells co-cultured with CD74-deficient DCs, indicating stronger cytotoxic potential (FIG. 11G). Thus, CD74 regulates the functionality of DCs in the TME.

### Example 10

### CD74 Binds the IL-1β and SP1 Promotors, which in Turn Regulate Tol-DCs and Breg Expansion

The mechanism of action of CD74 expressed on DC was investigated. TNBC cells were injected to the mice. Starting from day 10, mice were intravenously treated for 5 consecutive days (10-14), with either PBS or DRQ. DCs were sorted from the TME and purified RNA was then analyzed by RNA-seq. Inhibition of CD74 led to stronger DC activation, as seen by the upregulation of key pathways in the anti-tumor immune response, such as the cross-talk between DCs and NK cells [39, 40] or the classical markers of macrophage activation, which are essential for the anti-tumor response [41]. Moreover, pathways involved in metabolic signaling important for cancer progression, as the PPAR cascade, were downregulated (FIG. 23A).

Furthermore, as predicted the upstream regulators responsible for gene expression changes observed in the DRQ-treated mice. The IL-10 receptor was downregulated in DCs treated with the CD74 inhibitor (FIG. 23B). Analysis of the downstream events showed an elevation of a pro-inflammatory response, dictated mostly by the release of pro-inflammatory cytokines from DRQ-treated DCs (FIG. 23C). In addition, blocking CD74 resulted in a downregulation of cancer-related diseases and activation of the migration and interaction of the immune cells. Among the genes differentially expressed in DRQ-treated DCs, SP1 was downregulated, as confirmed by RT-qPCR (FIG. 12A), and IL-1β mRNA levels were upregulated under these conditions (FIG. 12B). The same effect was observed in DCs lacking CD74 (FIGS. 12C-12D).

SP1 is associated with immunosuppression in several cancer types, including TNBC [43, 44], and IL-1β is responsible for the activation of a pro-inflammatory pathway. Although IL-1β expression in the TME is related to cancer progression [45], DCs expressing IL-1β are more immunogenic and reduce the expansion of immune-suppressive cells [46].

To verify that IL-1β controls IL-10 expression in DCs and the immunosuppressive ME, WT DCs were cultured in presence of E0771 cells and incubated with either IL-1β or PBS. IL-1β inhibited IL-10⁺ DC expansion (FIG. 13A). Moreover, to determine whether IL-1β-stimulated DCs regulate Breg expansion, B cells were cultured together with the DCs previously activated with E0771 and treated with either IL-1β or vehicle. As shown in FIG. 13B, IL-1β treatment of DCs, diminished Breg expansion, confirming that IL-1β treatment rendered DCs more immunogenic, and consequently less capable of inducing Bregs. Thus, DCs have a direct effect on Breg expansion, a process that is attenuated by IL-1β release. Furthermore, to assess whether B cells are directly affected by IL-1β, B cells were cultured in the presence of E0771, and treated with either IL-1β or vehicle. As shown in FIG. 13C, IL-1β only slightly reduced Breg expansion. To determine whether the effect is DC-mediated, DCs were added to the B cell culture with E0771 cells. The presence of DCs together with B cells strongly upregulated IL-10 release, suggesting that DCs powerfully control the Breg expansion in the presence of cancer cells, and that IL-1β plays a key role in reducing IL-10 release (FIG. 13C).

CD74-ICD is a regulator of transcription in health and disease [20, 47]. To determine whether CD74 attenuates the expression of IL-1β in DCs, DCs derived from the TME were sorted and the binding of CD74-ICD to the promotor region of IL-1β was analyzed by Chromatin Immunoprecipitation qPCR (ChIP-qPCR). A significant enrichment of binding of CD74-ICD to the promotor area of IL-1β in DCs was detected (FIG. 13D), thus it regulates its transcription.

Next, the role of SP1 in the immunosuppressive environment was investigated. To verify that SP1 contributes to IL-10 secretion by DCs, WT DCs were cultured in the presence of E0771 cells and incubated with either the SP1 blocker, mithramycin (MIT), or DMSO. MIT treatment abrogated IL-10⁺ DC expansion, suggesting a direct correlation between SP1 and IL-10 release (FIG. 14A).

To determine whether SP1 can directly induce Breg expansion via DCs, B cells were cultured together with DCs previously activated with E0771, and treated with either MIT or DMSO control (FIG. 14B). MIT-treated DCs negatively affected Breg expansion, indicating that DCs control the Breg expansion, a process that is augmented by SP1. To assess the role of SP1 on B cells, these cells were cultured in presence of E0771 and treated with either MIT or DMSO. Blocking SP1 on B cells alone did not affect Breg expansion. However, addition of DCs to this culture enhanced the percentage of Bregs and therefore boosted the effect of MIT on IL-10 release (FIG. 14C). Finally, to determine whether CD74 regulates the transcription of SP1 in DCs, sorted DCs from the TME were activated for 1 h with either PBS or MIF, and binding of CD74-ICD to the promotor region of SP1 was determined by ChIP-qPCR analysis (FIG. 14D). MIF activation induced a significant enrichment of binding to the promotor regions of SP1 by CD74-ICD, indicating a direct effect on SP1 transcription regulation through the MIF-CD74 axis (FIG. 14E). Since IL-1β transcription results to be inhibited by CD74, it was determined whether SP1 as transcription factor, acts in a MIF-CD74 dependent manner, to control IL-1β expression. SP1 as a transcription factor has the ability to bind the IL-1β promotor upon MIF activation, downregulating its transcription, as shown in FIG. 14F. Blocking SP1, resulted in the upregulation of IL-1β (FIG. 14G). These results suggest that CD74-ICD in DCs binds the SP1 promotor, which regulates IL-1β and IL-10 release, and in turn, governs the expansion of the Breg population.

### REFERENCES

1. Waks A G, Winer E P. Breast Cancer Treatment: A Review. JAMA. 2019; 321 (3): 288-300. Epub 2019 Jan. 23. doi: 10.1001/jama.2018.19323. PubMed PMID: 30667505.
2. Stagg J, Allard B. Immunotherapeutic approaches in triple-negative breast cancer: latest research and clinical prospects. Ther Adv Med Oncol. 2013; 5 (3): 169-81. Epub 2013 May 2. doi: 10.1177/1758834012475152. PubMed PMID: 23634195; PubMed Central PMCID: PMCPMC3630481.
3. da Silva J L, Cardoso Nunes N C, Izetti P, de Mesquita G G, de Melo A C. Triple negative breast cancer: A thorough review of biomarkers. Crit Rev Oncol Hematol. 2020; 145:102855. Epub 2020 Jan. 14. doi: 10.1016/j.critrevonc.2019.102855. PubMed PMID: 31927455.
4. Yin L, Duan J J, Bian X W, Yu S C. Triple-negative breast cancer molecular subtyping and treatment progress. Breast Cancer Res. 2020; 22 (1): 61. Epub 2020 Jun. 11. doi: 10.1186/s13058-020-01296-5. PubMed PMID: 32517735; PubMed Central PMCID: PMCPMC7285581.
5. Yu T, Di G. Role of tumor microenvironment in triple-negative breast cancer and its prognostic significance. Chin J Cancer Res. 2017; 29 (3): 237-52. Epub 2017 Jul. 22. doi: 10.21147/j.issn.1000-9604.2017.03.10. PubMed PMID: 28729775; PubMed Central PMCID: PMCPMC5497211.
6. Anderson D A, 3rd, Dutertre C A, Ginhoux F, Murphy K M. Genetic models of human and mouse dendritic cell development and function. Nat Rev Immunol. 2021; 21 (2): 101-15. Epub 2020 Sep. 11. doi: 10.1038/s41577-020-00413-x. PubMed PMID: 32908299.
7. Binnewies M, Mujal A M, Pollack J L, Combes A J, Hardison E A, Barry K C, et al. Unleashing Type-2 Dendritic Cells to Drive Protective Antitumor CD4 (+) T Cell Immunity. Cell. 2019; 177 (3): 556-71 e16. Epub 2019 Apr. 9. doi: 10.1016/j.cell.2019.02.005. PubMed PMID: 30955881; PubMed Central PMCID: PMCPMC6954108.
8. Zong J, Keskinov A A, Shurin G V, Shurin M R. Tumor-derived factors modulating dendritic cell function. Cancer Immunol Immunother. 2016; 65 (7): 821-33. Epub 2016 Mar. 18. doi: 10.1007/s00262-016-1820-y. PubMed PMID: 26984847.
9. Rosser E C, Mauri C. Regulatory B cells: origin, phenotype, and function. Immunity. 2015; 42 (4): 607-12. doi: 10.1016/j.immuni.2015.04.005. PubMed PMID: 25902480.
10. Mauri C, Menon M. The expanding family of regulatory B cells. Int Immunol. 2015; 27 (10): 479-86. doi: 10.1093/intimm/dxv038. PubMed PMID: 26071023; PubMed Central PMCID: PMCPMC4587489.
11. Stumptner-Cuvelette P, Benaroch P. Multiple roles of the invariant chain in MHC class II function. Biochim Biophys Acta. 2002; 1542:1-13.
12. Naujokas M F, Morin M, Anderson M S, Peterson M, Miller J. The chondroitin sulfate form of invariant chain can enhance stimulation of T cell responses through interaction with CD44. Cell. 1993; 74:257-68.
13. Roche P A, Furuta K. The ins and outs of MHC class II-mediated antigen processing and presentation. Nat Rev Immunol. 2015; 15 (4): 203-16. Epub 2015 Feb. 28. doi: 10.1038/nri3818. PubMed PMID: 25720354; PubMed Central PMCID: PMCPMC6314495.
14. Matza D, Wolstein O, Dikstein R, Shachar I. Invariant Chain Induces B Cell Maturation by Activating TAFII105-NF-kB Dependent Transcription Program. J Biol Chem. 2001; 276:27203-6.
15. Matza D, Kerem A, Lantner F, Shachar I. Invariant chain induced B cell differentiation requires intramembrane-proteolytic release of the cytosolic domain. Immunity. 2002; 17:549-60.
16. Becker-Herman S, Arie G, Medvedovsky H, Kerem A, Shachar I. CD74 is a member of the regulated intramembrane proteolysis (RIP) processed protein family. Mol Biol Cell. 2005; 16:5061-9.
17. Starlets D, Gore Y, Binsky I, Haran M, Harpaz N, Shvidel L, et al. Cell Surface CD74 initiates a signaling cascade leading to cell proliferation and survival. Blood. 2006; 107:4807-16.
18. Lantner F, Starlets D, Gore Y, Flaishon L, Yamit-Hezi A, Dikstein R, et al. CD74 induces TAp63 expression leading to B cell survival. Blood. 2007; 110:4303-11. PubMed PMID: 17846227.
19. Gore Y, Starlets D, Maharshak N, Becker-Herman S, Kaneyuki U, Leng L, et al. Macrophage migration inhibitory factor (MIF) induces B cell survival by activation of a CD74/CD44 receptor complex. J Biol Chem. 2008; 283:2784-92. PubMed PMID: 18056708.
20. David K, Friedlander G, Pellegrino B, Radomir L, Lewinsky H, Leng L, et al. CD74 as a regulator of transcription in normal B cells. Cell Rep. 2022; 41 (5): 111572. Epub 2022 Nov. 3. doi: 10.1016/j.celrep.2022.111572. PubMed PMID: 36323260.
21. Meyer-Siegler K, Hudson P B. Enhanced expression of macrophage migration inhibitory factor in prostatic adenocarcinoma metastases. Urology. 1996; 48:448-52.
22. Bando H, Matsumoto G, Bando M, Muta M, Ogawa T, Funata N, et al. Expression of macrophage migration inhibitory factor in human breast cancer: association with nodal spread. Jpn J Cancer Res. 2002; 93:389-96.
23. Nishihira J, Ishibashi T, Fukushima T, Sun B, Sato Y, Todo S. Macrophage migration inhibitory factor (MIF): Its potential role in tumor growth and tumor-associated angiogenesis. Ann N Y Acad Sci. 2003; 995:171-82.
24. Meyer-Siegler K L, Leifheit E C, Vera P L. Inhibition of macrophage migration inhibitory factor decreases proliferation and cytokine expression in bladder cancer cells. BMC Cancer. 2004; 4:34-45.
25. Ishigami S, Natsugoe S, Tokuda K, Nakajo A, Iwashige H, Aridome K, et al. Invariant chain expression in gastric cancer. Cancer Lett. 2001; 168:87-91.
26. Young A N, Amin M B, Moreno C S, Lim S D, Cohen C, Petros J A, et al. Expression profiling of renal epithelial neoplasms: a method for tumor classification and discovery of diagnostic molecular markers. Am J Pathol. 2001; 158:1639-51.
27. Ioachim H L, Pambuccian S E, Hekimgil M, Giancotti F R, Dorsett B H. Lymphoid monoclonal antibodies reactive with lung tumors. Diagnostic applications. Am J Surg Pathol. 1996; 20:64-71.
28. Datta M W, Shahsafaei A, Nadler L M, Freeman G J, Dorfman D M. Expression of MHC class II-associated invariant chain (Ii; CD74) in thymic epithelial neoplasms. Appl Immunohistochem Mol Morphol. 2000; 8:210-5.
29. Lazova R, Moynes R, May D, Scott G. LN-2 (CD74). A marker to distinguish atypical fibroxanthoma from malignant fibrous histiocytoma. Cancer. 1997; 79:2115-24.
30. Narni F, Kudo J, Mars W, Calabretta B, Florine D L, Barlogie B, et al. HLA-DR-associated invariant chain is highly expressed in chronic lymphocytic leukemia. Blood. 1986; 68:372-7.
31. Veenstra H, Jacobs P, Dowdle E B. Abnormal association between invariant chain and HLA class II alpha and beta chains in chronic lymphocytic leukemia. Cell Immunol. 1996; 171:68-73.
32. Hasby E A, Khalifa R A. Expression of CD74 in invasive breast carcinoma: its relation to Nottingham Prognostic Index, hormone receptors, and HER2 immunoprofile. Tumori. 2017; 103 (2): 193-203. Epub 2016 Nov. 12. doi: 10.5301/tj.5000562. PubMed PMID: 27834468.
33. Tian B, Zhang Y, Li N, Liu X, Dong J. CD74: a potential novel target for triple-negative breast cancer. Tumour Biol. 2012; 33 (6): 2273-7. Epub 2012 Sep. 1. doi: 10.1007/s13277-012-0489-x. PubMed PMID: 22935920.
34. Mizue Y, Nishihira J, Miyazaki T, Fujiwara S, Chida M, Nakamura K, et al. Quantitation of macrophage migration inhibitory factor (MIF) using the one-step sandwich enzyme immunosorbent assay: elevated serum MIF concentrations in patients with autoimmune diseases and identification of MIF in erythrocytes. Int J Mol Med. 2000; 5:397-403.
35. Binsky I, Haran M, Starlets D, Gore Y, Lantner F, Harpaz N, et al. IL-8 secreted in a macrophage migration-inhibitory factor- and CD74-dependent manner regulates B cell chronic lymphocytic leukemia survival. Proc Natl Acad Sci USA. 2007; 104:13408-13. PubMed PMID: 17686984.
36. Lee H, Lee H J, Song I H, Bang W S, Heo S H, Gong G, et al. CD11c-Positive Dendritic Cells in Triple-negative Breast Cancer. In Vivo. 2018; 32 (6): 1561-9. Epub 2018 Oct. 24. doi: 10.21873/invivo. 11415. PubMed PMID: 30348717; PubMed Central PMCID: PMCPMC6365742.
37. Bucala R, Shachar I. The integral role of CD74 in antigen presentation, MIF signal transduction, and B cell survival and homeostasis. Mini Rev Med Chem. 2014; 14 (14): 1132-8. PubMed PMID: 25643611.
38. Meza-Romero R, Benedek G, Gerstner G, Kent G, Nguyen H, Offner H, et al. Increased CD74 binding and EAE treatment efficacy of a modified DRalpha1 molecular construct. Metab Brain Dis. 2019; 34 (1): 153-64. Epub 2018 Oct. 26. doi: 10.1007/s11011-018-0331-2. PubMed PMID: 30353480; PubMed Central PMCID: PMCPMC6364671.
39. Bodder J, Zahan T, van Slooten R, Schreibelt G, de Vries IJM, Florez-Grau G. Harnessing the cDC1-NK Cross-Talk in the Tumor Microenvironment to Battle Cancer. Front Immunol. 2020; 11:631713. Epub 2021 Mar. 9. doi: 10.3389/fimmu.2020.631713. PubMed PMID: 33679726; PubMed Central PMCID: PMCPMC7933030.
40. Kalinski P, Mailliard R B, Giermasz A, Zeh H J, Basse P, Bartlett D L, et al. Natural killer-dendritic cell cross-talk in cancer immunotherapy. Expert Opin Biol Ther. 2005; 5 (10): 1303-15. Epub 2005 Oct. 4. doi: 10.1517/14712598.5.10.1303. PubMed PMID: 16197336.
41. Choi J, Gyamfi J, Jang H, Koo J S. The role of tumor-associated macrophage in breast cancer biology. Histol Histopathol. 2018; 33 (2): 133-45. Epub 2017 Jul. 7. doi: 10.14670/HH-11-916. PubMed PMID: 28681373.
42. Alimirah F, Peng X, Yuan L, Mehta R R, von Knethen A, Choubey D, et al. Crosstalk between the peroxisome proliferator-activated receptor gamma (PPARgamma) and the vitamin D receptor (VDR) in human breast cancer cells: PPARgamma binds to VDR and inhibits 1alpha,25-dihydroxyvitamin D3 mediated transactivation. Exp Cell Res. 2012; 318 (19): 2490-7. Epub 2012 Aug. 14. doi: 10.1016/j.yexcr.2012.07.020. PubMed PMID: 22884583.
43. Zhao Y, Yao D, Li Y, Zhang S, Tao Z, Zhang L, et al. Loss of polarity protein Par3 is mediated by transcription factor Spl in breast cancer. Biochem Biophys Res Commun. 2021; 561:172-9. Epub 2021 May 24. doi: 10.1016/j.bbrc.2021.05.025. PubMed PMID: 34023783.
44. Chang C M, Lam HYP, Hsu H J, Jiang S J. Interleukin-10: A double-edged sword in breast cancer. Tzu Chi Med J. 2021; 33 (3): 203-11. Epub 2021 Aug. 14. doi: 10.4103/tcmj.tcmj_162_20. PubMed PMID: 34386356; PubMed Central PMCID: PMCPMC8323643.
45. Kaplanov I, Carmi Y, Kornetsky R, Shemesh A, Shurin G V, Shurin M R, et al. Blocking IL-1beta reverses the immunosuppression in mouse breast cancer and synergizes with anti-PD-1 for tumor abrogation. Proc Natl Acad Sci USA. 2019; 116 (4): 1361-9. Epub 2018 Dec. 14. doi: 10.1073/pnas.1812266115. PubMed PMID: 30545915; PubMed Central PMCID: PMCPMC6347724.
46. Ghiringhelli F, Apetoh L, Tesniere A, Aymeric L, Ma Y, Ortiz C, et al. Activation of the NLRP3 inflammasome in dendritic cells induces IL-1beta-dependent adaptive immunity against tumors. Nat Med. 2009; 15 (10): 1170-8. Epub 2009 Sep. 22. doi: 10.1038/nm.2028. PubMed PMID: 19767732.
47. Gil-Yarom N, Radomir L, Sever L, Kramer M P, Lewinsky H, Bornstein C, et al. CD74 is a novel transcription regulator. Proc Natl Acad Sci USA. 2017; 114 (3): 562-7. Epub 2016 Dec. 30. doi: 10.1073/pnas. 1612195114. PubMed PMID: 28031488; PubMed Central PMCID: PMCPMC5255621.
48. Yaddanapudi K, Rendon B E, Lamont G, Kim E J, Al Rayyan N, Richie J, et al. MIF Is Necessary for Late-Stage Melanoma Patient MDSC Immune Suppression and Differentiation. Cancer Immunol Res. 2016; 4 (2): 101-12. Epub 2015 Nov. 26. doi: 10.1158/2326-6066.CIR-15-0070-T. PubMed PMID: 26603621; PubMed Central PMCID: PMCPMC4740231.
49. Alban T J, Bayik D, Otvos B, Rabljenovic A, Leng L, Jia-Shiun L, et al. Glioblastoma Myeloid-Derived Suppressor Cell Subsets Express Differential Macrophage Migration Inhibitory Factor Receptor Profiles That Can Be Targeted to Reduce Immune Suppression. Front Immunol. 2020; 11:1191. Epub 2020 Jul. 7. doi: 10.3389/fimmu.2020.01191. PubMed PMID: 32625208; PubMed Central PMCID: PMCPMC7315581.
50. Cotzomi-Ortega I, Rosas-Cruz A, Ramirez-Ramirez D, Reyes-Leyva J, Rodriguez-Sosa M, Aguilar-Alonso P, et al. Autophagy Inhibition Induces the Secretion of Macrophage Migration Inhibitory Factor (MIF) with Autocrine and Paracrine Effects on the Promotion of Malignancy in Breast Cancer. Biology (Basel). 2020; 9 (1). Epub 2020 Jan. 23. doi: 10.3390/biology9010020. PubMed PMID: 31963754; PubMed Central PMCID: PMCPMC7169388.
51. Gai J W, Wahafu W, Song L, Ping H, Wang M, Yang F, et al. Expression of CD74 in bladder cancer and its suppression in association with cancer proliferation, invasion and angiogenesis in HT-1376 cells. Oncol Lett. 2018; 15 (5): 7631-8. Epub 2018 May 8. doi: 10.3892/ol.2018.8309. PubMed PMID: 29731899; PubMed Central PMCID: PMCPMC5920967.
52. Zheng Y X, Yang M, Rong T T, Yuan X L, Ma Y H, Wang Z H, et al. CD74 and macrophage migration inhibitory factor as therapeutic targets in gastric cancer. World J Gastroenterol. 2012; 18 (18): 2253-61. Epub 2012 May 23. doi: 10.3748/wjg.v18.i18.2253. PubMed PMID: 22611320; PubMed Central PMCID: PMCPMC3351777.
53. Figueiredo C R, Azevedo R A, Mousdell S, Resende-Lara P T, Ireland L, Santos A, et al. Blockade of MIF-CD74 Signalling on Macrophages and Dendritic Cells Restores the Antitumour Immune Response Against Metastatic Melanoma. Front Immunol. 2018; 9:1132. Epub 2018 Jun. 8. doi: 10.3389/fimmu.2018.01132. PubMed PMID: 29875777; PubMed Central PMCID: PMCPMC5974174.
54. Li X, Du H, Zhan S, Liu W, Wang Z, Lan J, et al. The interaction between the soluble programmed death ligand-1 (sPD-L1) and PD-1 (+) regulator B cells mediates immunosuppression in triple-negative breast cancer. Front Immunol. 2022; 13:830606. Epub 2022 Aug. 9. doi: 10.3389/fimmu.2022.830606. PubMed PMID: 35935985; PubMed Central PMCID: PMCPMC9354578.
55. Castiello L, Sabatino M, Ren J, Terabe M, Khuu H, Wood L V, et al. Expression of CD14, IL10, and Tolerogenic Signature in Dendritic Cells Inversely Correlate with Clinical and Immunologic Response to TARP Vaccination in Prostate Cancer Patients. Clin Cancer Res. 2017; 23 (13): 3352-64. Epub 2017 Jan. 12. doi: 10.1158/1078-0432.CCR-16-2199. PubMed PMID: 28073842; PubMed Central PMCID: PMCPMC5496805.
56. Mansilla M J, Contreras-Cardone R, Navarro-Barriuso J, Cools N, Berneman Z, Ramo-Tello C, et al. Cryopreserved vitamin D3-tolerogenic dendritic cells pulsed with autoantigens as a potential therapy for multiple sclerosis patients. J Neuroinflammation. 2016; 13 (1): 113. Epub 2016 May 22. doi: 10.1186/s12974-016-0584-9. PubMed PMID: 27207486; PubMed Central PMCID: PMCPMC4874005.
57. Kendal A R, Chen Y, Regateiro F S, Ma J, Adams E, Cobbold S P, et al. Sustained suppression by Foxp3+ regulatory T cells is vital for infectious transplantation tolerance. J Exp Med. 2011; 208 (10): 2043-53. Epub 2011 Aug. 31. doi: 10.1084/jem.20110767. PubMed PMID: 21875958; PubMed Central PMCID: PMCPMC3182049.
58. Nakamura K, Smyth M J. Myeloid immunosuppression and immune checkpoints in the tumor microenvironment. Cell Mol Immunol. 2020; 17 (1): 1-12. Epub 2019 Oct. 16. doi: 10.1038/s41423-019-0306-1. PubMed PMID: 31611651; PubMed Central PMCID: PMCPMC6952382.
59. Tulotta C, Ottewell P. The role of IL-1B in breast cancer bone metastasis. Endocr Relat Cancer. 2018; 25 (7): R421-R34. Epub 2018 May 16. doi: 10.1530/ERC-17-0309. PubMed PMID: 29760166; PubMed Central PMCID: PMCPMC5987176.
60. Mohd Jaya F N, Garcia S G, Borras F E, Chan GCF, Franquesa M. Paradoxical role of Breg-inducing cytokines in autoimmune diseases. J Transl Autoimmun. 2019; 2:100011. Epub 2020 Aug. 4. doi: 10.1016/j.jtauto.2019.100011. PubMed PMID: 32743499; PubMed Central PMCID: PMCPMC7388338.
61. Keren-Shaul H, Kenigsberg E, Jaitin D A, David E, Paul F, Tanay A, et al. MARS-seq2.0: an experimental and analytical pipeline for indexed sorting combined with single-cell RNA sequencing. Nat Protoc. 2019; 14 (6): 1841-62. Epub 2019 May 19. doi: 10.1038/s41596-019-0164-4. PubMed PMID: 31101904.

It will be apparent that the precise details of the methods or compositions described may be varied or modified without departing from the spirit of the described aspects of the disclosure. We claim all such modifications and variations that fall within the scope and spirit of the claims below.

We claim:

1. A method of treating a subject with cancer comprising administering to the subject a therapeutically effective amount of
a recombinant polypeptide comprising an antigenic peptide covalently linked to a DRα1 domain or portion thereof comprising a glutamine residue at a position corresponding to amino acid 50 of SEQ ID NO: 1 or SEQ ID NO: 2; or
a nucleic acid encoding the recombinant polypeptide.

2. The method of claim 1, wherein the recombinant polypeptide further comprises a linker between the antigenic peptide and the DRα1 domain.

3. The method of claim 2, wherein the linker comprises a first glycine-serine spacer, a thrombin cleavage site, and a second glycine-serine spacer.

4. The method of claim 1, wherein the antigenic peptide is myelin oligodendrocyte glycoprotein (MOG)-35-55 or myelin basic protein (MBP)-85-99.

5. The method of claim 4, wherein the MOG-35-55 is human or mouse MOG-35-55.

6. The method of claim 1, wherein the recombinant polypeptide comprises or consists of the amino acid sequence of SEQ ID NO: 1 or SEQ ID NO: 2.

7. The method of claim 1, wherein the subject is administered about 0.1 mg/kg to about 10 mg/kg of the recombinant polypeptide.

8. The method of claim 1, wherein the cancer is a solid tumor or a hematological malignancy.

9. The method of claim 8, wherein the solid tumor is melanoma, glioblastoma, or breast cancer.

10. The method of claim 1, wherein the cancer of the subject does not express a BRAF mutation.

11. The method of claim 10, wherein the cancer of the subject does not express a BRAF V600 mutation.

12. The method of claim 1, wherein the subject with cancer is resistant to immune checkpoint blockade therapy.

13. The method of claim 1, further comprising administering one or more additional therapies to the subject.

14. The method of claim 13, wherein the one or more additional therapies comprise one or more of surgery, radiation, chemotherapy, and immunotherapy.

15. The method of claim 14, wherein the immunotherapy comprises immune checkpoint blockade therapy.


SEQUENCE LISTING (5 sequences)

SEQ ID NO: 1 - protein, 120 aa, Synthetic construct
MEVGWYRPPFSRVVHLYRNGKGGGGSLVPRGSGGGGIKEEHVIIQAEFYQNPDQSGEFMFDFDGDEIFHVDMAKKETVWRLEEFGRFASFEAQGALANIAVDKANLEIMTKRSNYTPITN

SEQ ID NO: 2 - protein, 119 aa, Synthetic construct
MEVGWYRSPFSRVVHLYRNGKGGGGSLVPRGSGGGGIKEEHVIIQAEFYQNPDQSGEFMDFDGDEIFHVDMAKKETVWRLEEFGRFASFEAQGALANIAVDKANLEIMTKRSNYTPITN

SEQ ID NO: 3 - DNA, 363 bp, Synthetic construct
  source 1..363: mol_type = other DNA
atggaagttggttggtaccgtcccccgttctcccgtgttgttcacctgtaccgtaacggtaaaggaggtggaggctcactagtgccccgaggctctggaggtggaggcatcaaagaagaacatgtgatcatccaggccgagttctatcagaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaattaa

SEQ ID NO: 4 - DNA, 363 bp, Synthetic construct
  source 1..363: mol_type = other DNA
atggaagttggttggtaccgttccccgttctcccgtgttgttcacctgtaccgtaacggtaaaggaggtggaggctcactagtgccccgaggctctggaggtggaggcatcaaagaagaacatgtgatcatccaggccgagttctatcagaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaattaa

SEQ ID NO: 5 - protein, 14 aa, Synthetic construct
PVVHFFKNIVTPRT